(19) United States
(12) Patent Application Publication
(10) Pub. No.: US 2022/0111019 A1
(43) Pub. Date: Apr. 14, 2022

(54) BRCA1 MODULATING COMPOUNDS, FORMULATIONS THEREOF, AND USES THEREOF

(71) Applicant: VIRGINIA TECH INTELLECTUAL PROPERTIES, INC., BLACKSBURG, VA (US)

(72) Inventors: Deborah F. KELLY, University Park, PA (US); Yanping LIANG, Blacksburg, VA (US); William DEARNALEY, University Park, PA (US)

(21) Appl. No.: 17/278,267

(22) PCT Filed: Sep. 19, 2019
(86) PCT No.: PCT/US2019/051830

§ 371 Date: Mar. 19, 2021

Related U.S. Application Data
(60) Provisional application No. 62/733,385, filed on Sep. 19, 2018.

(51) Int. Cl. A61K 38/48; C12N 9/48; A61K 33/40; A61P 35/00

(52) U.S. Cl. CPC A61K 38/48; A61P 35/00; A61K 33/40; C12N 9/485

(57) ABSTRACT

Described herein are BRCA1 modulating compounds and formulations thereof. In some aspects, the BRCA1 modulating compound is a deubiquitinase. In some aspects, the BRCA1 modulating compound is a deubiquitinase inhibitor. as Also described herein are methods of treating a subject in need thereof with a BRCA1 modulating compound or formulation thereof. In some aspects, the subject in need thereof can have a cancer. In some aspects, the subject in need thereof has one or more BRCA1 mutations.

### CROSS-REFERENCE TO RELATED APPLICATIONS

This application claims the benefit of and priority to co-pending U.S. Provisional Patent Application No. 62/733,385, filed on Sep. 19, 2018, entitled “BRCA1 MODULATING COMPOUNDS, FORMULATIONS THEREOF, AND USES THEREOF,” the contents of which is incorporated by reference herein in its entirety.

### STATEMENT REGARDING FEDERALLY SPONSORED RESEARCH

This invention was made with Government support CA193578, CA227261, and CA219700 awarded by the National Institutes of Health. The Government has certain rights in the invention.

### BACKGROUND

Cancer is a world-wide heath concern that causes significant mortality and morbidity. As such there exists an urgent and unmet need for cancer treatments.

### SUMMARY

Described herein are aspects of a method of treating a cancer or symptom thereof in a subject in need thereof, the method comprising: administering an amount of a BRCA1 modulating compound or pharmaceutical formulation thereof to the subject in need thereof. In some aspects, the BRCA1 modulating compound is a deubiquitinase. In some aspects, the deubiquitinase is selected from the group of: USP2, USP1, USP3, USP4, USP5, USP6, USP7, USP8, USP9X, USP9Y, USP10, USP11, USP12, USP13, USP14, USP15, USP16, USP17, USP17L2, USP17L3, USP17L4, USP17L5, USP17L7, USP17L8, USP18, USP19, USP20, USP21, USP22, USP23, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, USP46, OTUB1, OTUB2, ATXN3, ATXN3L, BAP1, UCHL1, UCHHL3, UCHL5, and any combination thereof. In some aspects, the BRCA1 modulating compound is a deubiquitinase inhibitor. In some aspects, the deubiquitinase inhibitor is selected from the group of: ML364, P022077, P5091, Cpd 14, P22077, HBX 41,108, HBX-19,818, HBX-28,258, HBX 90,397, Ethyloxyimino-9H-indeno [1,2-b] pyrazine-2,3-dicarbonitrile, IU1, Isatin O-acyl oxime deriatives, LDN91946, LS1, NSC112200, NSC267309, PR-619, 15-Deoxy-α_{12,14} prostaglandin J2, b-AP15, RA-9, F6, G5, WP1130, Eeyarestatin-1, Curcumin, AC17, Gambogic acid, LDN-57444, GW7647, pimozide, 12Δ-PGJ2, AM146, RA-14, betulinic acid, and any combination thereof. In some aspects, the method further includes administering a compound to the subject that increases the oxidative stress of a cell or a population thereof in the subject. In some aspects, the compound that increases the oxidative stress of a cell or a population thereof is hydrogen peroxide. In some aspects, the cancer is a breast cancer. In some aspects, the cancer is an ovarian cancer. In some aspects, the cancer is a brain cancer. In some aspects, the cancer is a cancer that has or is at least in part caused by a mutated BRCA1. In some aspects, the amount of BRCA1 modulating compound or formulation thereof ranges from about 0.1 μg/kg to about 1000 mg/kg.

Also described herein is the use of a BRCA1 modulating compound for the treatment of a cancer or a symptom thereof.

Also described herein is the use of a BRCA1 modulating compound in the manufacture of a medicament for treatment of cancer or a symptom thereof.

As described herein are aspects of a pharmaceutical formulation that can include: an effective amount of a BRCA1 modulating compound; and a pharmaceutically acceptable carrier. In some aspects, the BRCA1 modulating compound is a deubiquitinase. In some aspects, the deubiquitinase is selected from the group of: USP2, USP1, USP3, USP4, USP5, USP6, USP7, USP8, USP9X, USP9Y, USP10, USP11, USP12, USP13, USP14, USP15, USP16, USP17, USP17L2, USP17L3, USP17L4, USP17L5, USP17L7, USP17L8, USP18, USP19, USP20, USP21, USP22, USP23, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, USP46, OTUB1, OTUB2, ATXN3, ATXN3L, BAP1, UCHL1, UCHHL3, UCHL5, and any combination thereof. In some aspects, the BRCA1 modulating compound is a deubiquitinase inhibitor. In some aspects, the deubiquitinase inhibitor is selected from the group of: ML364, P022077, P5091, Cpd 14, P22077, HBX 41,108, HBX-19,818, HBX-28,258, HBX 90,397, Ethyloxyimino-9H-indeno [1,2-b] pyrazine-2,3-dicarbonitrile, IU1, Isatin O-acyl oxime deriatives, LDN91946, LS1, NSC112200, NSC267309, PR-619, 15-Deoxy-α_{12,14} prostaglandin J2, b-AP15, RA-9, F6, G5, WP1130, Eeyarestatin-1, Curcumin, AC17, Gambogic acid, LDN-57444, GW7647, pimozide, 12Δ-PGJ2, AM146, RA-14, betulinic acid and any combination thereof. In some aspects, the pharmaceutical formulation further comprises an amount of a compound capable of increasing oxidative stress in a population of cells in the subject.

These and other aspects, objects, features, and advantages of the example embodiments will become apparent to those having ordinary skill in the art upon consideration of the following detailed description of example embodiments.

### BRIEF DESCRIPTION OF THE DRAWINGS

Further aspects of the present disclosure will be readily appreciated upon review of the detailed description of its various embodiments, described below, when taken in conjunction with the accompanying drawings. The figures herein are for illustrative purposes only and are not necessarily drawn to scale.

FIGS. 1A-1F can show that the wild type BRCA1-BARD1 EM structure resembles a clamp-like motif. (FIG. 1A) BRCA1 and BARD1 protein sequences show the N-terminal RING domains and C-terminal BRCT motifs. BRCA1 also contains central nuclear localization sequences (NLS). (FIG. 1B) Phosphorylated BRCA1 migrates at about 260 kDa while BARD1 migrates at about 87 kDa according to SDS-PAGE. Western blots of co-IP experiments identified interactions between BRCA1 and BARD1. (FIG. 1C) Image with inset of wild type BRCA1-BARD1 (left) and corresponding 2D class averages (center). Scale bar is 50 nm. Projections of the 3D structure (right) agree with class averages. Box size is 25 nm. (FIG. 1D) The BRCA1-BARD1 EM map shows a clamp-like motif (movie S1). Atomic models for the BRCA1-BARD1 RING domain (magenta; pdbcode, 1JM7 (18)) and the BRCT domain of BRCA1 (gray; pdbcode, 1JNX (19)) were fit the EM density based on antibody-labeling procedures (FIGS. 5A-5E, movie S1). Scale bar is 1.5 nm. Cross-sections through the RING domain show the quality of the model fit. (FIG. 1E) 8-OxoG formation (red) in the nuclei (blue) of HCC70 cells after treating with 1 mM H₂O₂ for 40-minute. Untreated cells (—H₂O₂) did not accumulate 8-OxoG. Scale bar is 50 μm. (FIG. 1F) Western blots indicated relatively stable levels of BRCA1, BARD1, and RAD51 in HCC70 cells (70), and in cells resistant to oxidative stress (70R) during H₂O₂ treatment. Nuclear β-actin served as a loading control. Immunoblot (IB); input material (IN); unbound/depleted material (DEP); Interacting proteins (IP*).

FIGS. 2A-2D can show that the BRCA1_{5382insC}-BARD1 structure shows subtle variations from the wild type structure. (FIG. 2A) The protein sequence of BRCA1_{5382insC} has a frameshift mutation at residue S1755 (red star). (FIG. 2B) BRCA1_{5382insC} migrates at ˜260 kDa and BARD1 migrates at about 87 kDa according to SDS-PAGE. Western blots of co-IP experiments identified interactions between mutated BRCA1 and BARD1. (FIG. 2C) Image with inset of BRCA1_{5382insC}-BARD1 (left) and corresponding 2D class averages (center). Scale bar is 50 nm. Projections of the 3D structure (right) agree with the class averages. Box size is 25 nm. (FIG. 2D) The 3D structure of BRCA1_{5382insC}-BARD1 reveals a clamp-like motif with defined RING and BRCT regions (movie S2). Scale bar is 1.5 nm. Molecular models for the RING domain of BRCA1-BARD1 (magenta; pdbcode, 1JM7 (18)) and a homology model of the BRCT domain (25) fit in the envelope. Red star indicates the mutation site. Scale bar is 1.5 nm. Cross-sections through the RING domain region show the model fit (FIGS. 6A-6D, movie S2). Immunoblot (IB); input material (IN); unbound/depleted material (DEP); immunoprecipitated proteins (IP*).

FIGS. 3A-3F can demonstrate changes in the BRCA1_{5382insC}-BARD1 EM structure under oxidative pressure. (FIG. 3A) Image (left) and class averages (center) of mutated BRCA1_{5382insC}-BARD1 isolated from HCC1937 cells treated with 1 mM H₂O₂. Scale bar is 50 nm. Projections of the 3D structure (right) agree with the class averages. Box size of averages is 25 nm. (FIG. 3B) Under oxidative conditions, BRCA1_{5382insC} migrates at about 270 kDa and BARD1 migrates at about 87 kDa according SDS-PAGE and western blots analysis (FIG. 3C) Following H₂O₂ treatment, the RING domain of BRCA1_{5382insC} was difficult to detect compared to wild type BRCA1 (WT). Wild type BRCA1 (WT-R) from treated HCC70-R cells is resistant to oxidative damage. Nuclear β-actin served as a loading control. (FIG. 3D) The BRCA1_{5382insC}-BARD1 structure shows a clamp-like motif with extra density adjacent to the RING domain (black circle). Scale bar is 1.5 nm. Atomic models for the RING domain (magenta; pdbcode, 1JM7 (18)) and a homology model of the BRCT domain (25) fit in the molecular envelope (FIGS. 7A-7F, movie S3). Difference peak (yellow) indicates the additional mass present in the hot spot region of BRCA1_{5382insC} under oxidative conditions. This additional mass was not present in the untreated BRCA1_{5382insC}-BARD1 density map (gray). Red star indicates the mutation site. Scale bar is 1.5 nm. Cross-sections through the RING domain are indicated (movie S3). (FIG. 3E) 8-OxoG formation (red) in the nuclei (blue) of HCC1937 cells treated for for 40-minute with 1 mM H₂O₂. Untreated cells (−H₂O₂) had inherent 8-OxoG foci. Scale bar is 50 μm. (FIG. 3F) Western blots indicated unstable BRCA1_{5382insC} and BARD1 compared to RAD51 in treated HCC1937 cells. Nuclear β-actin served as a loading control.

FIGS. 4A-4D can demonstrate that deubiquitinase treatment of BRCA1_{5382insC}-BARD1 restores structural integrity. (FIG. 4A) Western blot analysis of USP2-treated protein fractions isolated from HCC1937 cells experiencing oxidative stress. The band shift for BRCA1_{5382insC} to about 260 kDa in USP2-treated samples was confirmed by probing the BRCT and RING domains of BRCA1. Greater signal for the RING domain was detected in the USP2-treated samples along with a reduced signal for ubiquitin attachments at about 260 kDa. Increased levels of mono-ubiquitin (about 8 kDa) were found in USP2-treated samples. (FIG. 4B) Image (left) and class averages (center) of mutated BRCA1_{5382insC}-BARD1 treated with 1 mM H₂O₂ and USP2. Scale bar is 50 nm. Projections of the 3D structure (right) are in good agreement with the class averages. Box size of averages is 25 nm. (FIG. 4C) The EM structure of BRCA1_{5382insC}-BARD1 shows a clamp-like motif lacking extra density adjacent to the RING domain (black circle) (FIG. 8A-8D, movie S4). Scale bar is 1.5 nm. (FIG. 4D) Difference peak (yellow) indicates the additional mass present in the hot spot region of BRCA1_{538insC} under oxidative conditions. This area of extra mass is lacking in the USP2-treated BRCA1_{5382insC}-BARD1 structure (green).

FIGS. 5A-5E. Biochemical characterization of wild type (WT) BRCA1-BARD1. (FIG. 5A) Western blot detection of co-immunoprecipitation (co-IP) experiments identified interactions between BRCA1 (about 260 kDa) and BARD1 (about 87 kDa). (FIG. 5B) Image with inset of wild type BRCA1-BARD1 particles and 2D class averages. Scale bar is 50 nm. Box size is 25 nm. (FIG. 5C) Antibodies (Abs, white arrows) against the BRCA1 RING or BRCT domains helped identify their location in individual particles. Contour maps highlight the density for attached antibodies. Antibody attachment sites were mapped to the 3D structure. Box size is 25 nm. Scale bar is 5 nm. (FIG. 5D) The angular distribution of particle orientations is not limited in the reconstruction. Fourier shell correlation (FSC) curve indicates the resolution of the density map is ˜14.5 Å for n=4008 particles. (FIG. 5E) Western blot detection of BRCA1, BARD1, and RAD51 in nuclear fractions showed relatively stable protein levels in (+) H₂O₂-treated HCC70 cells and HCC70-R cells. Untreated (˜H₂O₂) cells were not exposed to oxidative reagents. Nuclear β-actin served as a loading control. Immunoblot (IB); Input (IN), Interacting proteins (IP); Unbound/depleted material (DEP).

FIGS. 6A-6D. Biochemical characterization of mutated BRCA1_{5382insC}-BARD1. (FIG. 6A) Western blot detection of co-IP experiments identified interactions between BRCA1_{5382insC} (about 260 kDa) and BARD1 (about 87 kDa). (FIG. 6B) Image with inset of BRCA1_{5382insC}-BARD1 particles and 2D class averages. Scale bar is 50 nm. Box size is 25 nm. (FIG. 6C) 3D reconstruction of the BRCA1_{5382insC}-BARD1 heterodimer is shown in different orientations (movie S2). (FIG. 6D) The angular distribution of particle orientations is not limited in the reconstruction. FSC curve indicates the resolution of the density map is about 14.7 Å for n=4222 particles. Immunoblot (IB); Input (IN), Interacting proteins (IP); Unbound/depleted material (DEP).

FIGS. 7A-7F. Changes in the properties of the BRCA1_{5382insC}-BARD1 under oxidative conditions. (FIG. 7A) The RING domain of BRCA1_{5382insC} in H₂O₂-treated HCC1937 cells was difficult to detect compared to wild type BRCA1 (WT) in H₂O₂-treated cells. Wild type BRCA1 (WT-R) was also accessed in treated cells. Nuclear β-actin served as a loading control. (FIG. 7B) Image and class averages of mutated BRCA1_{5382insC}-BARD1 isolated from cells treated with 1 mM H₂O₂. Scale bar is 50 nm. Box size of averages is 25 nm. (FIG. 7C) 3D reconstruction of BRCA1_{5382insC}-BARD1 formed under oxidative conditions and displayed in different orientations (movie S3). Extra density in the hot spot region adjacent to the RING domain is circled in black. Scale bar is 1.5 nm. Atomic models for the RING domain (magenta; pdbcode, 1JM7 (18)) and a homology model of the BRCT domain (25) fit in the molecular envelope. (FIG. 7D) The angular distribution plot of particle orientations is not limited. (FIG. 7E) FSC curve indicates the resolution of the density map is about 15.6 Å for n=4103 particles. (FIG. 7F) Western blots showed unstable levels of BRCA1_{5382insC} (about 270 kDa) and BARD1 (about 87 kDa) compared to RAD51 (about 37 kDa) in H₂O₂-treated HCC1937 cells. Nuclear β-actin (about 42 kDa) served as a loading control.

FIGS. 8A-8D can demonstrate that the BRCA1_{5382insC}-BARD1 structure is restored following USP2 treatment. (FIG. 8A) Western blot analysis of USP2-treated protein fractions show a band shift for BRCA1_{5382insC} to about 260 kDa. The shift was detected using antibodies against the BRCT and RING domains of BRCA1. The RING domain was more easily detected in the USP2-treated samples. A reduced signal for ubiquitin attachments around about 260 kDa corresponded with increased levels of mono-ubiquitin (about 8 kDa) in USP2-treated samples. (FIG. 8B) Image and class averages of mutated BRCA1_{5382insC}-BARD1 treated with USP2. Scale bar is 50 nm. Box size of averages is 25 nm. (FIG. 8C) 3D reconstruction of USP2-treated BRCA1_{5382insC}-BARD1 formed under oxidative conditions and displayed in different orientations (movie S4). (FIG. 8D) Angular distribution plot shows a lack of preferred particle orientations. FSC curve indicates the resolution of the density map is about 15.4 Å for n=4000 particles.

FIGS. 9A-9B show flow charts of image processing procedures including steps for assessing particle heterogeneity during 2D averaging (FIG. 9A) and 3D classification (FIG. 9B) procedures.

FIG. 10 shows a schematic that showing mutated BRCA1 modification through ubiquitination. Mutated BRCA1-BARD1 (gray, mesh) lacks density in a lysine-rich hotspot region in comparison to ubiquitinated BRCA1-BARD1 (yellow, mesh). Upon enzymatically removing ubiquitin adducts from the complex, the bulky density in the hotspot area disappears. The to the treated structure is referred to herein as “restored” BRCA1-BARD1 (green, mesh). An atomic model of the ubiquitin monomer (pdb code, 1UBQ (33)) fits well within the difference density. Scale bar is 15 Å.

FIGS. 11A-11E can demonstrate that p53_{R306} forms active tetramers on native DNA. (FIG. 11A) Western blot analysis under denaturing conditions shows differences in the presence of p53_{R306} tetramers (about 160 kDa) and monomers (about 40 kDa) in nuclear extracts of HCC1937 cells. Cells were treated with H₂O₂ (Ox) and nuclear fractions were supplemented with USP2 and incubated at 4° C. or 37° C. for activation. Control cells lacked H₂O₂ treatment. (*) indicates activated samples with restored BRCA1. (FIG. 11B) Quantification of p53 tetramer to monomer (T/M) ratios in active and inactive extracts. (FIG. 11C) Coomassie-stained SDS-PAGE gel shows the purified p53_{R306} monomer obtained from H₂O₂-treated cells. Western blot shows the p53_{R306} tetramer assembly (about 160 kDa) and monomer (about 40 kDa). (FIG. 11D) EM image of the purified p53_{R306} assemblies along with class averages and corresponding projections of the 3D density map. Scale bar is 200 Å. Box size is 250 Å. (FIG. 11E) EM map (white) and homology model of the p53_{R306} tetramer (yellow) bound to DNA (blue) containing a double-stranded break (DSB). Scale bar is 15 Å.

FIGS. 12A-12E can demonstrate that restored BRCA1 collaborates with p53 in cancer cells. (FIG. 12A) Restored BRCA1 migrates at about 260 kDa and BARD1 migrates at about 87 kDa on a denaturing gel. (FIG. 12B) Western blot analysis and densitometry measurements showed changes in ubiquitinated-p53 (about 68 kDa) in reaction mixtures receiving restored BRCA1-BARD1 (μg). Controls (−) lacked restored BRCA1-BARD1. (FIG. 12C) Quantitative increases (μg) were noted in the total signal for ubiquitinated-p53 bands compared with control samples (−). (FIG. 12D) Wild-type p53 (p53_{WT}) isolated from U87MG cells migrated at about 50 kDa according to SDS-PAGE and Western blot analysis. p53_{WT} tetramers migrated at about 220 kDa on a native (non-denaturing) gel. EM image and class averages of p53-DNA assemblies (about 80 Å) showed similar features as the mutated assemblies (about 70 Å). Calculated projections of the density map for p53_{WT} assemblies were in good agreement with experimental averages. Scale bar is 200 Å. Box size is 250 Å. (FIG. 12E) EM map (white) of the p53_{WT} tetramer (yellow) engaging DNA (blue) in a pre- or post-repair configuration. Scale bar is 15 Å.

FIGS. 13A-13E can demonstrate that breast cancer cells are weakened by DUB inhibitors. (FIG. 13A) The IC₅₀ value for the DUB inhibitor, ML364, use in HCC1937 cells is ˜7-10 μM. (FIG. 13B) Cancer cells (HCC1937 line) treated H₂O₂, an oxidizing reagent (Ox), and subsequently with ML364 (+) showed a decline in viability. (FIG. 13C) The 8-Oxo-Guanine (8-Oxo-G) accumulation (red punctate) was more abundant in the nucleus (blue) of ML364-treated cells than in H₂O₂-treated cells. Cells treated with H₂O₂ and ML364 showed the greatest 8-Oxo-G buildup in an around the nucleus. Scale bar is 50 μm. (FIG. 13D) Western blot analysis of cell lysates show a shift in the migration of BRCA1 along with changes in its band intensity following H₂O₂ and ML364 (Ox/+ML364) treatment. Corresponding changes in the levels of p53_{R306} tetramers were noted in lysate fractions of treated cells. (FIG. 13E) Quantification of Western blot band intensities showed that dual treatment (Ox/+ML364) lowered mutated BRCA1 and p53_{R306} levels along with the propensity for DNA repair.

FIGS. 14A-14D can demonstrate that ubiquitinated BRCA1 from breast cancer cells can be structurally restored. (FIG. 14A) The EM density map of ubiquitinated BRCA1-BARD1 with a BRCT mutation (red star) was computed using the RELION software package (18). Atomic models for the RING domain (pdb code, 1JM7 (8)) and the truncated BRCT fit well in the map. An ubiquitin monomer (pdb code, 1UBQ (33)) was placed in the identified hotspot density. (FIG. 14B) Ubiquitinated BRCA1-BARD1 loses density in the hotspot region upon USP2 treatment. We refer to the treated structure as “restored”. (FIG. 14C) Western blots reveal ubiquitinated-BRCA1 (control) migrates slower (about 270 kDa) than USP2-treated BRCA1 (about 260 kDa) as previously reported (10). Free ubiquitin (about 8 kDa) increased in USP2-treated samples relative to untreated controls. (FIG. 14D) Density map of the restored BRCA1-BARD1 complex lacks ubiquitin density in the hotspot area. Scale bar is 15 Å.

FIGS. 15A-15D can demonstrate an EM analysis of mutated p53_{R306} tetramer assembly reveals double-strand breaks in native DNA. (FIG. 15A) EM image of truncated p53_{R306} isolated from breast cancer cells (HCC1937 line). Corresponding class averages and projections of the EM density map show good agreement. Scale bar is 200 Å; box size is 250 Å. (FIG. 15B) Cross-sections through the p53_{R306} reconstruction (white) show the tetramer core (yellow; pdb code, 2AC0 (21)) bound to double stranded DNA breaks (blue). Scale bar is 15 Å and the diameter of the complex is about 70 Å. (FIG. 15C) Angular distribution plot indicates particle orientations are not limited in the image data. (FIG. 15D) Fourier shell correlation (FSC) curve indicates the resolution of the density map is about 15.5 Å for n=747 particles. C2-symmetry was imposed during the refinement procedure, bringing the total number of particles to an equivalency of 1494.

FIGS. 16A-16C can demonstrate results from a biochemical analysis of BRCA1-deficient cancer cells. (FIG. 16A) Western blot analysis of BRCA1 detection in cell lysates derived from normal human astrocytes (control) in comparison to the glioma cells (T98G and A172 line). Low BRCA1 levels (BRCAₗₒ) were detected in T98G cells and high BRCA1 levels (BRCAₕᵢ) were detected in A172 cells. In each of these cell lines, BRCA1 migrated at about 260 kDa. Nuclear β-actin (about 42 kDa) served as a loading control. (FIG. 16B) Western blot analysis of nuclear reaction mixtures prepared from T98G cells and supplemented with increasing quantities (μg) of restored BRCA1-BARD1 (+). Increased levels of ubiquitined-p53 (about 68 kDa) were detected using antibodies against the K63-ubiquitin linkages (K63-Ubq). Nuclear β-actin (about 42 kDa) served as a loading control. (FIG. 16C) Western blots show differences in p53 migration in a non-modified (about 50 kDa) and ubiquitinated state (about 68 kDa).

FIGS. 17A-17D can demonstrate the results of an EM analysis of wild-type p53 tetramers isolated from human cancer cells reveals its association with native DNA. (FIG. 17A)

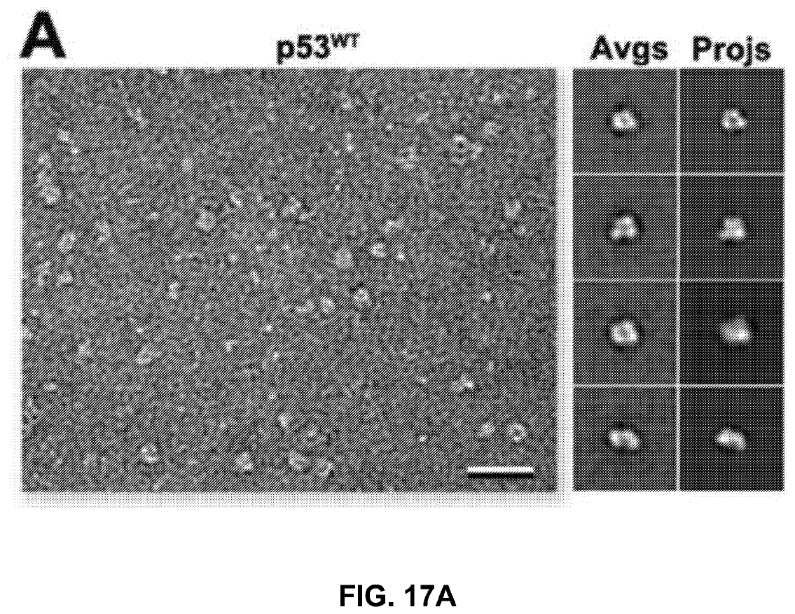

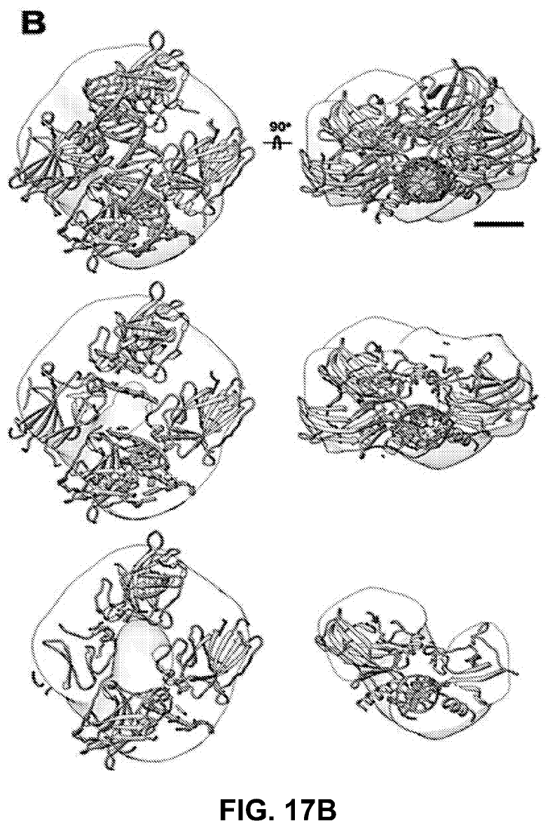

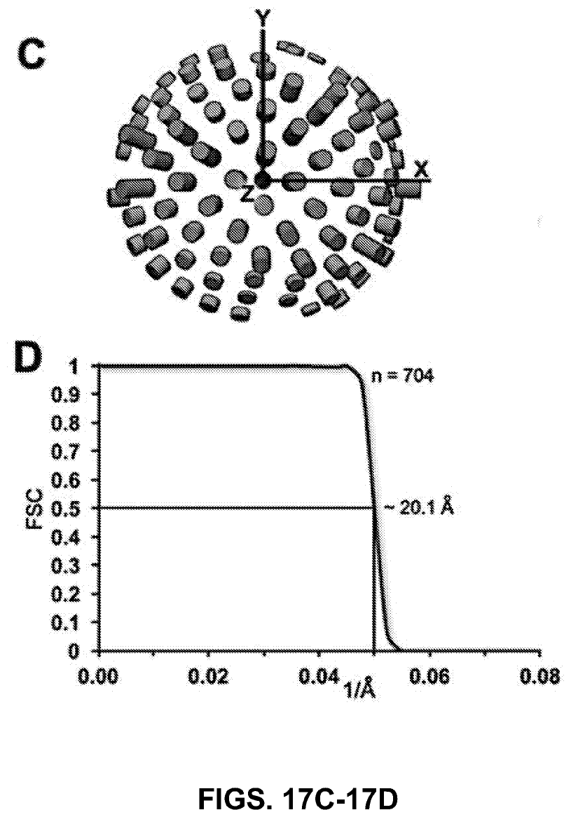

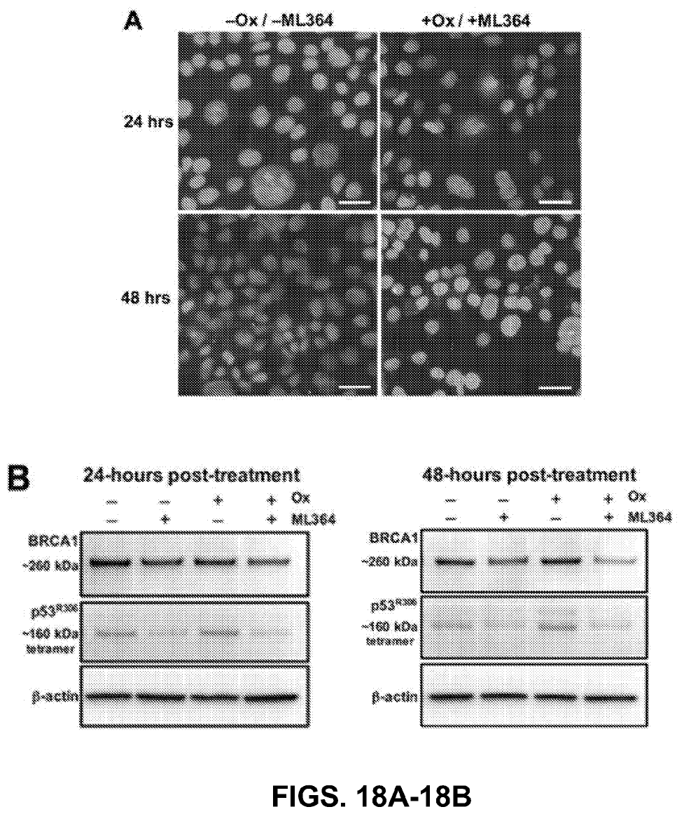

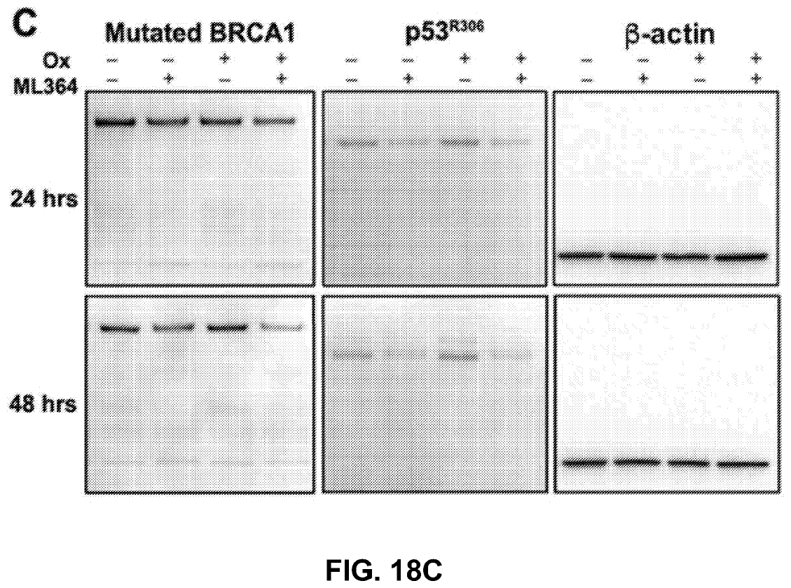

EM Image of wild-type p53 (p53_{WT}) derived from human glioma cells (U87MG line). Class averages and projections calculated from the EM density map are in good agreement. Scale bar 200 Å, Box size, 250 Å. (FIG. 17B) Cross-sections of the p53_{WT} density map (white) show the model for the tetramer core (yellow; pdb code, 2AC0 (21)) bound to native DNA (blue) during a pre- or post-repair state. Scale bar is 15 Å and the diameter of the complex is about 80 Å. (FIG. 17C) Angular distribution plot of particle orientations shows a non-limited view of their orientations in the images. (FIG. 17D) FSC curve indicates the resolution of the density map is ˜20 Å for n=704 particles. C2-symmetry was implemented during refinement procedures, bringing the total number of particles to an equivalency of 1408.

FIGS. 18A-18D can demonstrate results from fluorescence imaging and western blot detection show that DUB inhibitors can attenuate repair of oxidative DNA in cancer cells. (FIG. 18A) Immunofluorescent images were acquired for HCC1937 cells treated with H₂O₂ (+Ox) and/or the DUB inhibitor (ML364) in its IC₅₀ range. Within 24-hours post-treatment, 8-Oxo-G accumulation (red punctuate) in the cell nucleus (blue) was detected. A higher level of 8-Oxo-G was detected in cells treated with H₂O₂ and ML364 (+0x/+ML364) than in controls (−Ox/−ML364), indicating a reduced capacity for oxidative DNA damage repair. This trend continued for up to 48 hours. (FIG. 18B) Western blots were performed on cell lysates of HCC1937 cells treated with H₂O₂ (+0x) and/or the DUB inhibitor (ML364) in its IC₅₀ range after 24 and 48 hours of treatment. Notable shifts in band intensity and migration patterns in mutated BRCA1 (about 260 kDa) and p53_{R306} tetramers (about 160 kDa) during the combined treatment corresponded with decreased tumor suppressor levels in comparison to control (−Ox/−ML364) conditions. Nuclear β-actin (about 42 kDa) served as a loading control. (FIG. 18C) Whole western blot membranes for the analysis shown in (FIG. 18B).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2_{nd} edition (1989) (Sambrook, Fritsch, and Maniatis); Molecular Cloning: A Laboratory Manual, 4ₜₕ edition (2012) (Green and Sambrook); Current

Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboraotry Manual, 2_{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2_{nd} edition (2011).

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed. Citation or identification of any document in this application is not an admission that such a document is available as prior art to the present invention.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the singular forms “a”, “an”, and “the” include both singular and plural referents unless the context clearly dictates otherwise.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +/−10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

Embodiments of the present disclosure will employ, unless otherwise indicated, techniques of molecular biology, microbiology, organic chemistry, biochemistry, physiology, cell biology, cancer biology, and the like, which are within the skill of the art. Such techniques are explained fully in the literature.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible unless the context clearly dictates otherwise.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Definitions

As used herein, “active agent” or “active ingredient” can refer to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseo us, intraocular, intracranial, intraperitoneal, intralesional, intranasal, intracardiac, intraarticular, intracavernous, intrathecal, intravireal, intracerebral, and intracerebroventricular, intratym panic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques.

As used herein, “agent” can refer to any substance, compound, molecule, and the like, which can be biologically active or otherwise can induce a biological and/or physiological effect on a subject to which it is administered to. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “anti-infective” can refer to compounds or molecules that can either kill an infectious agent or inhibit it from spreading. Anti-infectives include, but are not limited to, antibiotics, antibacterials, antifungals, antivirals, and antiprotozoans.

The term “biocompatible”, as used herein, refers to a material that along with any metabolites or degradation products thereof that are generally non-toxic to the recipient and do not cause any significant adverse effects to the recipient. Generally speaking, biocompatible materials are materials that do not elicit a significant inflammatory or immune response when administered to a patient. Biocompatibility, as used herein, can be quantified using the following in vivo biocompatibility assay. A material or product is considered biocompatible if it produces, in a test of biocompatibility related to immune system reaction, less than 50%, 45%, 40%, 35%, 30%, 25%, 20%, 15%, 10%, 8%, 6%, 5%, 4%, 3%, 2%, or 1% of the reaction, in the same test of biocompatibility, produced by a material or product the same as the test material or product except for a lack of the surface modification on the test material or product. Examples of useful biocompatibility tests include measuring and assessing cytotoxicity in cell culture, inflammatory response after implantation (such as by fluorescence detection of cathepsin activity), and immune system cells recruited to implant (for example, macrophages and neutrophils).

The term “biodegradable” as used herein, generally refers to a material that will degrade or erode under physiologic conditions to smaller units or chemical species that are capable of being metabolized, eliminated, or excreted by the subject. The degradation time is a function of composition and morphology. Degradation times can be from hours to weeks.

As used herein “cancer” can refer to one or more types of cancer including, but not limited to, acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, astrocytomas, atypical teratoid/Rhabdoid tumors, basa cell carcinoma of the skin, bile duct cancer, bladder cancer, bone cancer (including but not limited to Ewing Sarcoma, osteosarcomas, and malignant fibrous histiocytoma), brain tumors, breast cancer, bronchial tumors, Burkitt lymphoma, carcinoid tumor, cardiac tumors, germ cell tumors, embryonal tumors, cervical cancer, cholangiocarcinoma, chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, endometrial cancer, ependymoma, esophageal cancer, esthesioneuroblastoma, extracranial germ cell tumor, extragonadal germ cell tumor, eye cancer (including, but not limited to, intraocular melanoma and retinoblastoma), fallopian tube cancer, gallbladder cancer, gastric cancer, gastrointestinal carcinoid tumor, gastrointestinal stromal tumors, central nervous system germ cell tumors, extracranial germ cell tumors, extragonadal germ cell tumors, ovarian germ cell tumors, testicular cancer, gestational trophoblastic disease, Hairy cell leukemia, head and neck cancers, hepatocellular (liver) cancer, Langerhans cell histiocytosis, Hodgkin lymphoma, hypopharyngeal cancer, islet cell tumors, pancreatic neuroendocrine tumors, kidney (renal cell) cancer, laryngeal cancer, leukemia, lip cancer, oral cancer, lung cancer (non-small cell and small cell), lymphoma, melanoma, Merkel cell carcinoma, mesothelioma, metastatic squamous cell neck cancer, midline tract carcinoma with and without NUT gene changes, multiple endocrine neoplasia syndromes, multiple myeloma, plasma cell neoplasms, mycosis fungoides, myelodyspastic syndromes, myelodysplastic/myeloproliferative neoplasms, chronic myelogenous leukemia, nasal cancer, sinus cancer, non-Hodgkin lymphoma, pancreatic cancer, paraganglioma, paranasal sinus cancer, parathyroid cancer, penile cancer, pharyngeal cancer, pheochromocytoma, pituitary cancer, peritoneal cancer, prostate cancer, rectal cancer, Rhabdomyosarcoma, salivary gland cancer, uterine sarcoma, Sézary syndrome, skin cancer, small intestine cancer, large intestine cancer (colon cancer), soft tissue sarcoma, T-cell lymphoma, throat cancer, oropharyngeal cancer, nasopharyngeal cancer, hypoharyngeal cancer, thymoma, thymic carcinoma, thyroid cancer, transitional cell cancer of the renal pelvis and ureter, urethral cancer, uterine cancer, vaginal cancer, cervical cancer, vascular tumors and cancer, vulvar cancer, and Wilms Tumor.

As used herein, “chemotherapeutic agent” or “chemotherapeutic” can refer to a therapeutic agent utilized to prevent or treat cancer.

As used herein, “concentrated” can refer to a molecule or population thereof, including but not limited to a polynucleotide, peptide, polypeptide, protein, antibody, or fragments thereof, that is distinguishable from its naturally occurring counterpart in that the concentration or number of molecules per volume is greater than that of its naturally occurring counterpart.

As used herein, “control” can refer to an alternative subject or sample used in an experiment for comparison purpose and included to minimize or distinguish the effect of variables other than an independent variable.

The term “copolymer” as used herein, generally refers to a single polymeric material that is comprised of two or more different monomers. The copolymer can be of any form, such as random, block, graft, etc. The copolymers can have any end-group, including capped or acid end groups.

As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “deoxyribonucleic acid (DNA)” and “ribonucleic acid (RNA)” can generally refer to any polyribonucleotide or polydeoxribonucleotide, which may be unmodified

RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA) or coding mRNA (messenger RNA).

As used herein, “dose,” “unit dose,” or “dosage” can refer to physically discrete units suitable for use in a subject, each unit containing a predetermined quantity of the BRCA1 or mutated BRCA1 modulating compound and/or a pharmaceutical formulation thereof calculated to produce the desired response or responses in association with its administration.

As used herein, “effective amount” refers to the amount of a compound provided herein that is sufficient to effect beneficial or desired biological, emotional, medical, or clinical response of a cell, tissue, system, animal, or human. An effective amount can be administered in one or more administrations, applications, or dosages. The term cam also include within its scope amounts effective to enhance or restore to substantially normal physiological function. The “effective amount” can refer to the amount of a BRCA1 modulating compound or formulation thereof described herein that can kill and/or inhibit a cancer cell and/or growth and/or proliferation thereof; increase ubiquitination in a cell, such as a cancer cell; and/or decrease the amount of ubiquitin on a mutated BRCA1.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

The term “hydrophilic”, as used herein, refers to substances that have strongly polar groups that are readily soluble in water.

The term “hydrophobic”, as used herein, refers to substances that lack an affinity for water; tending to repel and not absorb water as well as not dissolve in or mix with water.

The term “lipophilic”, as used herein, refers to compounds having an affinity for lipids.

The term “molecular weight”, as used herein, can generally refer to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “organism”, “host”, and “subject” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans).

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” can refer to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutically acceptable salt” can refer to any acid or base addition salt whose counter-ions are non-toxic to the subject to which they are administered in pharmaceutical doses of the salts.

As used herein, “positive control” can refer to a “control” that is designed to produce the desired result, provided that all reagents are functioning properly and that the experiment is properly conducted.

As used herein, “polypeptides” or “proteins” can refer to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, V\/), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be required for the structure, function, and regulation of the body's cells, tissues, and organs.

As used herein, the term “specific binding” can refer to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10₋₃ M or less, 10₋₄ M or less, 10₋₅ M or less, 10₋₆ M or less, 10₋₇ M or less, 10₋₈ M or less, 10₋₉ M or less, 10₋₁₀ M or less, 10₋₁₁ M or less, or 10₋₁₂ M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10₋₃ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used interchangeably herein, the terms “sufficient” and “effective,” can refer to an amount (e.g. mass, volume, dosage, concentration, and/or time period) needed to achieve one or more desired result(s). For example, a therapeutically effective amount refers to an amount needed to achieve one or more therapeutic effects.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a cancer (e.g. a breast cancer, an ovarian cancer, a cancer that has and/or is caused in whole or at least in part by a BRCA1 mutation, a cancer that has and/or is caused at least in part by the BRCA1_{5382insC} mutation). The effect can be therapeutic in terms of a partial or complete cure of a disease, condition, symptom or adverse effect attributed to the disease, disorder, or condition. The term “treatment” as used herein covers any treatment of a cancer (e.g. a breast cancer, an ovarian cancer, a cancer that has and/or is caused at least in part by a BRCA1 mutation, a cancer that has and/or is caused at least in part by the BRCA1_{5382insC} mutation)., in a subject, particularly a human, and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

Discussion

The breast cancer type 1 susceptibility protein (BRCA1) is a tumor suppressor gene located on chromosome 17. BRCA1 coordinates DNA repair through a variety of mechanisms designed to protect genetic material. BRCA1 performs these functions in association with its binding partner the BRCA1-associated Ring Domain protein (BARD1). In the nucleus of cells, the BRCA1-BARD1 heterodimer interacts with other repair proteins at DNA lesions to function as an E3-ubiquitin ligase. Through a series of controlled steps the BRCA1-BARD1 complex facilitates the transfer of ubiquitin moieties to a variety of nuclear protein substrates. These ubiquitin adducts direct their bound substrates toward different fates, one of which involves correcting DNA damage. Base-excision repair (BER) is a process that corrects non-helix distorting damage to DNA caused by conditions such as oxidation. BRCA1 plays an essential role in helping cells deal with oxidative conditions by triggering BER pathways through ubiquitin signaling.

Many mutations have been identified in BRCA1, some of which are harmful and can cause cancer. Some of these mutations are responsible for hereditary breast-ovarian cancer syndrome. Although mutations in BRCA1 (or BRCA2) are implicated in only about 5-10 percent of breast cancers, the impact on women with mutations in one or both of these genes is more profound. Women with harmful mutations in BRCA1 and/or BRCA2 have a risk of developing breast cancer that is about five times the normal risk and have a risk of developing ovarian cancer that is about ten to about thirty times the normal risk. Women with a high-risk BRCA1 mutation have a greater risk of developing cancer than those with a BRCA2 mutation.

Some of these mutations, including those in BRCA1 can disable important DNA repair processes, such as BER and others. Cells harboring inherited mutations in the BRCA1 gene cannot adequately deal with increased levels of reactive oxygen species (ROS) arising from estrogen metabolism. These inadequacies can lead to functional deficiencies in BER, an accumulation of DNA insults, and widespread genomic instability—a known hallmark of cancer induction. As such, there exists a need for improved treatments for BRCA1 mediated cancers.

With that said, described herein are compositions and formulations that can modify mutated BRCA1. Also described herein are methods of treating a cell with a mutated BRCA1, such as a cancer cell, that can include the step of administering to a cell, population of cells, and/or a subject in need thereof, an amount of a deubiquitinase. Also described herein are methods of treating a cell with a mutated BRCA1, such as a cancer cell, that can include the step of administering to a cell, population of cells, and/or a subject in need thereof, an amount of a deubiquitinase inhibitor. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

BRCA1 Modulating Pharmaceutical Formulations

Cells with a mutation in the BRCA1 have difficulty protecting their genetic material from repeated daily stressors. These deficiencies create an unstable environment in the nucleus as DNA insults accumulate. Some of these mutations, including those in BRCA1 can disable important DNA repair processes, such as BER and others. Cells harboring inherited mutations in the BRCA1 gene cannot adequately deal with increased levels of reactive oxygen species

(ROS) arising from estrogen metabolism. These inadequacies can lead to functional deficiencies in BER, an accumulation of DNA insults, and widespread genomic instability—a known hallmark of cancer induction.

One such clinical mutation, BRCA1_{5382insC}, influences the manner in which BRCA1 itself is modified in cancer cells. The main type of modification identified on BRCA1_{5382insC} was K48-linked ubiquitin chains. In the nucleus of cancer cells, higher levels of ubiquitination correlated with lower levels of active BRCA1_{5382insC} and changes in its biochemical properties. This site, termed the “degron” sequence, is proximal to the BRCA1 N-terminal RING domain. Excessive ubiquitination of the mutated BRCAI in this “hotspot” region inappropriately targets the mutated BRCA1 for degradation, leading to and/or contributing to the reduced BRCA1 levels and/or activity noted in cancer cells harboring the BRCA1_{5382insC}.

Described herein are pharmaceutical formulations that can contain an amount of a BRCA1 modulating compound and a pharmaceutically acceptable carrier. The BRCA1 modulating compound can be a deubiquitinase or a deubiquitinase inhibitor. The BRACA1 modulating compound can be effective to modulate a characteristic, such as an activity or functionality, of the BRCA1 and/or a mutated BRCA1 (e.g. a BRCA1_{5382insC}).

The deubiqutinase can be effective to remove excessive ubiquination on a mutated BRCA1, and can be capable of restoring the structure of a mutated BRCA1 and/or its DNA repair and other functions. The deubiquitinase inhibitor can inhibit the removal of ubiquitin on BRCA1, such as a mutated BRCA1. In cancer cells, which are rapidly growing and dividing, more so than healthy cells. The deubiquitinase inhibitor can generate or enhance a deficiency in BRCA1 in the cell, thus impairing the cells warning and repair systems, of the rapidly dividing cancer cells and causing cancer cell death. As stated above, reducing the ability of BRCA1 to repair DNA, e.g. through a mutation, can contribute to the development of cancer. As such, it is counter intuitive that inhibiting DNA repair by increasing ubiquination of BRCA1 would facilitate treating a BRCA1 mediated cancer.

The amount of deubiquitinase can be an amount effective to treat a cancer or a symptom thereof. The cancer can be a cancer caused at least in part by a mutated BRCA1. The mutated BRCA1 can have one or more BRCA1 mutations. Many BRCA1 mutations are known in the art. There are over 1600 known mutations in BRCA1. Exemplary BRCA1 mutations include, but are not limited to BRCA1_{5382insC}, BRCA1_{185delAG}, BRCA1_{3819del5}, and BRCA1_{4153delA}, IVS7+36T>C, IVS7+38T>C, IVS7+41C>T, IVS7+49del15, IVS16-68G>A, IVS16−92G>A, IVS18+65G>A, c.2196G>A, c.3232A>G, c.3667A>G, c.4956A>G, c.5075G>A, c.5095C>T, 4216-2nt A→G, 3172ins5, 2594delC, 1806C>T, 1201del11, 5370C>T, 1675delA and 1135insC, 1135insA, 1675delA, 816delGT, 3203del11, 3347delAG, G5193A, Exon 13 and 22 del, 2804delAA, IVS2011G>A, IVS21-36del510, 5382insC, 1411insT, 2138delA 2312del5, 2457C4T, 185insA, 185delAG, G4956A, 4184del4, intron 5 splice, C4446T, 3875del4, 2800delAA, 2080delA, 2594delC, 655A>G, 4282delAG, 300T>G, 4184del4bp, c.3700_3704del5, (exon 17 and 22 del Exon 13dup), 4446C>T, 2953delGTAinsC, R1443X, 3875dekGTCT, 3600del11, G1710X, Exons 8-13 del, Exons 3-8 and 18-20 dup, c.3228_3229delAG, c.3285delA, c.1377_1378insA c.5062_5064delTGT, Exons 17, 9-19, 18-19, 1a-2, 16-20 del, 4843delC, IVS5+3A>G, E1221X, 2478-2479insG, 330A>G, c.187_188delAG, c.5385insC, c.5242C>A, c.66_68delAG, c.5123C>A, c.1961delA, c.3770_3771delAG, and c.5152+5G>A, Exons 3-5 del, 5382insC, 300T>G, 185delAG, 3819del5, c.190T>C, 2991del5, C5370T, 3875del4, AluSx ins (g.17686-17695), AluY ins (g.18760-18769), AluJb ins (g.33248-33276), Exons 21-24 del, Exons 5-14 del, exons 1-17 del, c.3700_3704del5, c.843_846del4, c.4243delG, 461delTC, G1738R, G5331A, 3819delGTAAA, c.1193C>A, c.181T>G, c.1687C>T, c.844_850dupTCATTAC, 1806C>T, IVS16-2A>G, c.116G>A, c.844_850dupTCATTAC, c.1687C>T, 2795del4, C1806T, c.3318C>A, c.4790C>A, C61G, 3447del4, C61G, 5429delG, 3232A.G, 4956A.G, c.5231delT, C3522T, 3450delCAAG, A1708E (polymorphism), Exons 9-12 del, IVS13+1G>A, 4730insG, T5443G, IVS16+6T>C, 943ins10, c.951_952insA, c.1129_1135insA, c.4603G>T and IVS20+1G>A, Exons 1-2 del, c.3124_3133delAGCAATATTA, c.2805_2808delAGAT, 5622C>T, c.307T>A, 509C>A, c.2333delC, c.4065_4068delTCAA 3746_3747insA (c.3627_3628insA), 5199G>T (c.5080G>T), 3478del5, 5589del8, 1100delAT, 2778G>A, 3552C>T, exon 10 dup, 5,273G>A, c.470_471delCT, c.3342_3345delAGAA, c.5406+1_5406+3delGTA and c.981_982delAT, 2845A>T, 3300delA, T320G, c.5191C>A, Exon 13 dup, 13-15 del, c.2845insA, 4427T>C, 2846insA, 2201C>T and 4956A>G (79%), 3668A>G, 2731C>T, 3232A.G, 3667A.G, exon 3 dup, 4627C>A, 4184del4, 2080insA, IVS14-1G>A, 2041insA, 4284delAG, 3889delAG, 2388delG, g.-1075C>G, g.-235A>G, g.-134T>C, g.442-34C>T, g.548-58delT, c.2077G>A, c.2082C>T, c.2311T>C, c.2612C>T, c.3113A>G, c.3119G>A, c.3548A>G, c.4308T>C, c.4837A>G, g.4987-68A>G, g.4987-92A>G, g.5075-53C>T, g.5152+66G>A, g.381_389del9ins29, g. 421G>T, g. 1286C>T, IVS16-92A>G, IVS16-68A>G, 4837A>G, IVS18+65G>A, Tyr978X, IVS17-53C>T, g.381-389del9ins29, G2031T, 2983C>A, 3450delCAAG, c.3548A>G, c.-26G>A, c.317-54C>G, 5341T>G, 5364C>G, 5379 G>T, 1014DelGT, 3889DelAG, c.3086delT, c.5404delG, c.856T>G, IVS17-2A>T, 5454delC, 3300delA, A1708E, 981delAT, C61G, Exon 21 del (c.5277+480_5332+672del), intron 20 (AluSg), intron 21 (AluY), 738C>A, 330 dupA (novel), 4160 delAG, the 2789 delG, 5385 insC, c.4041delAG, c.2551delG and c.5266dupC, c.798_799delTT, c.46_74del29, c.1016dupA, c.5095C>T, c.4942A>T, c.2805delA/2924delA, c.1504_1508del, and combinations thereof (see e.g. Wang et al. 2012. Mol. Biol. Rep. 39(3): 2109-2118 and Karami and Mehdipour. 2013. BioMed Research Int. pp. 1-21). In some aspects, the cancer can be a cancer caused in whole or at least apart by a BRCA1 mutation. The cancer can be a cancer caused at least in part by a BRCA1_{5382insC}. In some aspects, the cancer can be a breast cancer, ovarian cancer, a brain cancer, pancreatic cancer, or a combination thereof. The amount of deubiquitinase can be an amount effective to reduce the amount of ubiquitin on a mutated BRCA1. The mutated BRCA1 can have excessive ubiquitination. The mutated BRCA1 can be BRCA1_{5382insC}. The amount of deubiquitinase can be an amount effective to restore the function BRCA1_{5382insC} to substantially normal levels. For example, pharmaceutical formulation containing an amount of deubiquitinase can be effective to increase the response of p53, which is a substrate for BRCA1, in cells having a BRCA1_{5382insC} mutation. The deubiquitinase can be any deubiquitinase, which is a protease that is capable of cleaving the peptide or isopeptide bond between ubiquitin and its substrate protein. Suitable deubiquitinases include, but are not limited to, USP2, USP1, USP3, USP4, USP5, USP6, USP7, USP8, USP9X, USP9Y, USP10, USP11, USP12, USP13, USP14, USP15, USP16, USP17, USP17L2, USP17L3, USP17L4, USP17L5, USP17L7, USP17L8, USP18, USP19, USP20, USP21, USP22, USP23, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, USP46, OTUB1, OTUB2, ATXN3, ATXN3L, BAP1, UCHL1, UCHHL3, UCHL5, and any combination thereof. Cells having a BRCA1_{5382insC} mutation can be identified using any suitable assay capable of detecting said mutation. Suitable assays include any polynucleotide-based assay, including but not limited to PCR-based assays. Such suitable assays will be instantly appreciated by one of ordinary skill in the art in view of this discussion.

The amount of deubiquitinase inhibitor can be an amount effective to treat a cancer or a symptom thereof. The cancer can be a cancer caused at least in part by a mutated BRCA1. The cancer can be a cancer caused at least in part by a BRCA1_{5382insC} mutation. The cancer can be a breast cancer. The cancer can be an ovarian cancer. The amount of deubiquitinase inhibitor can be an amount effective to increase the ubiquitination of BRCA1 and other proteins in a cell. The amount of deubiquitinase inhibitor can be an amount effective to decrease the DNA repair mechanisms of the cell. The amount of deubiquitinase inhibitor can be an amount effective to kill a cancer cell. Suitable deubiquitinase inhibitors can include, but are not limited to, ML364, P022077, P5091, Cpd 14, P22077, HBX 41,108, HBX-19,818, HBX-28,258, HBX 90,397, Ethyloxyimino-9H-indeno [1,2-b] pyrazine-2,3-dicarbonitrile, IU1, Isatin O-acyl oxime deriatives, LDN91946, LS1, NSC112200, NSC267309, PR-619, 15-Deoxy-α_{12,14} prostaglandin J2, b-AP15, RA-9, F6, G5, WP1130, Eeyarestatin-1, Curcumin, AC17, Gambogic acid, LDN-57444, GW7647, pimozide, 12Δ-PGJ2, AM146, RA-14, and betulinic acid.

The BRCA1 modulating compounds and/or formulations thereof described herein can be administered to a subject. The subject can have or is suspected of having a cancer. The cancer can be a breast cancer. The cancer can be an ovarian cancer. The subject can have a BRCA1 mutation. The subject can have a BRCA1_{5382insC} mutation. The cancer can have a BRCA1 mutation. The cancer can have a BRCA1_{5382insC} mutation. The subject can be a subject in need thereof. The compounds and formulations described herein can be administered by a suitable route, such as but not limited to oral, infusion, and intravenous. Other suitable routes are described elsewhere herein. The BRCA1 modulating compounds and/or formulations thereof described herein can be used as a medicament for the treatment of a cancer, such as a cancer that has and/or is caused at least in part by a BRCA1 mutation (e.g. breast or ovarian cancer).

Parenteral Formulations

The BRCA1 modulating compounds described herein can be formulated for parenteral delivery, such as injection or infusion, in the form of a solution or suspension. The formulation can be administered via any route, such as, the blood stream or directly to the organ or tissue to be treated.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the BRCA1 modulating compounds described herein can be prepared in water or another solvent or dispersing medium suitably mixed with one or more pharmaceutically acceptable excipients including, but not limited to, surfactants, dispersants, emulsifiers, pH modifying agents, and combination thereof.

Suitable surfactants can be anionic, cationic, amphoteric or nonionic surface active agents. Suitable anionic surfactants include, but are not limited to, those containing carboxylate, sulfonate and sulfate ions. Suitable anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Suitable cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Suitable nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation can also contain an antioxidant to prevent degradation of the BRCA1 modulating compounds.

The formulation can be buffered to a pH of 3-8 for parenteral administration upon reconstitution. In some aspects, the pH of the formulation can be a pH of about 7.0-7.4 upon reconstitution. Suitable buffers include, but are not limited to, phosphate buffers, acetate buffers, and citrate buffers.

Water-soluble polymers can be used in the formulations for parenteral administration. Suitable water-soluble polymers include, but are not limited to, polyvinylpyrrolidone, dextran, carboxymethylcellulose, and polyethylene glycol. Sterile injectable solutions can be prepared by incorporating the BRCA1 modulating compounds in the desired amount in the appropriate solvent or dispersion medium with one or more of the excipients listed above, as required, followed by filtered sterilization. Dispersions can be prepared by incorporating the various sterilized BRCA1 modulating compounds into a sterile vehicle which contains the basic dispersion medium and the required other ingredients from those listed above. Sterile powders for the preparation of sterile injectable solutions can be prepared by vacuum-drying and freeze-drying techniques, which yields a powder of the BRCA1 modulating compounds with or without any additional desired ingredient from a previously sterile-filtered solution thereof. The powders can be prepared in such a manner that the particles are porous in nature, which can increase dissolution of the particles. Methods for making porous particles are well known in the art.

Pharmaceutical formulations for parenteral administration can be in the form of a sterile aqueous solution or suspension of the BRCA1 modulating compounds. Acceptable solvents include, for example, water, Ringer's solution, phosphate buffered saline (PBS), and isotonic sodium chloride solution. The formulation can also be a sterile solution, suspension, or emulsion in a nontoxic, parenterally acceptable diluent or solvent such as 1,3-butanediol.

In some instances, the formulation can be distributed or packaged in a liquid form. In other embodiments, formulations for parenteral administration can be packed as a solid, obtained, for example by lyophilization of a suitable liquid formulation. The solid can be reconstituted with an appropriate carrier or diluent prior to administration.

Solutions, suspensions, or emulsions for parenteral administration can be buffered with an effective amount of buffer necessary to maintain a pH suitable for ocular administration. Suitable buffers include, but are not limited to, acetate, borate, carbonate, citrate, and phosphate buffers.

Solutions, suspensions, or emulsions for parenteral administration can also contain one or more tonicity agents to adjust the isotonic range of the formulation. Suitable tonicity agents include, but are not limited to, glycerin, mannitol, sorbitol, sodium chloride, and other electrolytes.

Solutions, suspensions, or emulsions for parenteral administration can also contain one or more preservatives to prevent bacterial contamination of the ophthalmic preparations. Suitable preservatives include, but are not limited to, polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

Solutions, suspensions, or emulsions, use of nanotechnology including nanoformulations for parenteral administration can also contain one or more excipients, such as dispersing agents, wetting agents, and suspending agents.

Topical Formulations

The BRCA1 modulating compounds can be formulated for topical administration. Suitable dosage forms for topical administration include creams, ointments, salves, sprays, gels, lotions, emulsions, liquids, and transdermal patches. The formulation can be formulated for transmucosal, transepithelial, transendothelial, or transdermal administration. The topical formulations can contain one or more chemical penetration enhancers, membrane permeability agents, membrane transport agents, emollients, surfactants, stabilizers, and combination thereof.

In some embodiments, the BRCA1 modulating compounds can be administered as a liquid formulation, such as a solution or suspension, a semi-solid formulation, such as a lotion or ointment, or a solid formulation. In some embodiments, the BRCA1 modulating compounds can be formulated as liquids, including solutions and suspensions, such as eye drops or as a semi-solid formulation, such as ointment or lotion for topical application to the skin, to the mucosa, such as the eye, to the vagina, or to the rectum.

The formulation can contain one or more excipients, such as emollients, surfactants, emulsifiers, penetration enhancers, and the like.

Suitable emollients include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In some embodiments, the emollients can be ethylhexylstearate and ethylhexyl palmitate.

Suitable surfactants include, but are not limited to, emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In some embodiments, the surfactant can be stearyl alcohol.

Suitable emulsifiers include, but are not limited to, acacia, metallic soaps, certain animal and vegetable oils, and various polar compounds, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In some embodiments, the emulsifier can be glycerol stearate.

Suitable classes of penetration enhancers include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols).

Suitable emulsions include, but are not limited to, oil-in-water and water-in-oil emulsions. Either or both phases of the emulsions can include a surfactant, an emulsifying agent, and/or a liquid non-volatile non-aqueous material. In some embodiments, the surfactant can be a non-ionic surfactant. In other embodiments, the emulsifying agent is an emulsifying wax. In further embodiments, the liquid non-volatile non-aqueous material is a glycol. In some embodiments, the glycol is propylene glycol. The oil phase can contain other suitable oily pharmaceutically acceptable excipients. Suitable oily pharmaceutically acceptable excipients include, but are not limited to, hydroxylated castor oil or sesame oil can be used in the oil phase as surfactants or emulsifiers.

Lotions containing the BRCA1 modulating compounds are also described herein. In some embodiments, the lotion can be in the form of an emulsion having a viscosity of between 100 and 1000 centistokes. The fluidity of lotions can permit rapid and uniform application over a wide surface area. Lotions can be formulated to dry on the skin leaving a thin coat of their medicinal components on the skin's surface.

Creams containing the BRCA1 modulating compounds are also described herein. The cream can contain emulsifying agents and/or other stabilizing agents. In some embodiments, the cream is in the form of a cream having a viscosity of greater than 1000 centistokes, typically in the range of 20,000-50,000 centistokes. Creams, as compared to ointments, can be easier to spread and easier to remove.

One difference between a cream and a lotion is the viscosity, which is dependent on the amount/use of various oils and the percentage of water used to prepare the formulations.

Creams can be thicker than lotions, can have various uses, and can have more varied oils/butters, depending upon the desired effect upon the skin. In some embodiments of a cream formulation, the water-base percentage can be about 60% to about 75% and the oil-base can be about 20% to about 30% of the total, with the other percentages being the emulsifier agent, preservatives and additives for a total of 100%.

Ointments containing the BRCA1 modulating compounds and a suitable ointment base are also provided. Suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

Also described herein are gels containing the BRCA1 modulating compounds, a gelling agent, and a liquid vehicle. Suitable gelling agents include, but are not limited to, modified celluloses, such as hydroxypropyl cellulose and hydroxyethyl cellulose; carbopol homopolymers and copolymers; thermoreversible gels and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents can be selected for their ability to dissolve the drug. Other additives, which can improve the skin feel and/or emolliency of the formulation, can also be incorporated. Such additives include, but are not limited, isopropyl myristate, ethyl acetate, C₁₂-C₁₅ alkyl benzoates, mineral oil, squalane, cyclomethicone, capric/caprylic triglycerides, and combinations thereof.

Also described herein are foams that can include the BRCA1 modulating compounds. Foams can be an emulsion in combination with a gaseous propellant. The gaseous propellant can include hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or can become approved for medical use are suitable. The propellants can be devoid of hydrocarbon propellant gases, which can produce flammable or explosive vapors during spraying. Furthermore, the foams can contain no volatile alcohols, which can produce flammable or explosive vapors during use.

Buffers can be used to control pH of a composition. The buffers can buffer the composition from a pH of about 4 to a pH of about 7.5, from a pH of about 4 to a pH of about 7, or from a pH of about 5 to a pH of about 7. In some embodiments, the buffer can be triethanolamine.

Preservatives can be included to prevent the growth of fungi and microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

In certain embodiments, the formulations can be provided via continuous delivery of one or more formulations to a patient in need thereof. For topical applications, repeated application can be done or a patch can be used to provide continuous administration of the noscapine analogs over an extended period of time.

Enteral Formulations

The BRCA1 modulating compounds can be prepared in enteral formulations, such as for oral administration. Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations containing the BRCA1 modulating compounds can be prepared using pharmaceutically acceptable carriers. As generally used herein “carrier” includes, but is not limited to, diluents, preservatives, binders, lubricants, disintegrators, swelling agents, fillers, stabilizers, and combinations thereof. Polymers used in the dosage form include, but are not limited to, suitable hydrophobic or hydrophilic polymers and suitable pH dependent or independent polymers. Suitable hydrophobic and hydrophilic polymers include, but are not limited to, hydroxypropyl methylcellulose, hydroxypropyl cellulose, hydroxyethyl cellulose, carboxy methylcellulose, polyethylene glycol, ethylcellulose, microcrystalline cellulose, polyvinyl pyrrolidone, polyvinyl alcohol, polyvinyl acetate, and ion exchange resins. “Carrier” also includes all components of the coating composition which can include plasticizers, pigments, colorants, stabilizing agents, and glidants.

Formulations containing the BRCA1 modulating compounds can be prepared using one or more pharmaceutically acceptable excipients, including diluents, preservatives, binders, lubricants, disintegrators, swelling agents, fillers, stabilizers, and combinations thereof.

Delayed release dosage formulations containing the BRCA1 modulating compounds can be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on excipients, materials, equipment and process for preparing tablets and capsules and delayed release dosage forms of tablets, capsules, and granules. These references provide information on carriers, materials, equipment and process for preparing tablets and capsules and delayed release dosage forms of tablets, capsules, and granules.

The formulations containing the BRCA1 modulating compounds can be coated with a suitable coating material, for example, to delay release once the particles have passed through the acidic environment of the stomach. Suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings can be formed with a different ratio of water soluble polymer, water insoluble polymers and/or pH dependent polymers, with or without water insoluble/water soluble non polymeric excipient, to produce the desired release profile. The coating can be performed on a dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Additionally, the coating material can contain conventional carriers such as plasticizers, pigments, colorants, glidants, stabilization agents, pore formers and surfactants. Optional pharmaceutically acceptable excipients include, but are not limited to, diluents, binders, lubricants, disintegrants, colorants, stabilizers, and surfactants.

Diluents, also referred to as “fillers,” can be used to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful.

Binders can impart cohesive qualities to a solid dosage formulation, and thus can ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxpropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders.

Lubricants can be included to facilitate tablet manufacture. Suitable lubricants include, but are not limited to, magnesium stearate, calcium stearate, stearic acid, glycerol behenate, polyethylene glycol, talc, and mineral oil. A lubricant can be included in a tablet formulation to prevent the tablet and punches from sticking in the die. The lubricant can be chosen from such slippery solids as talc, magnesium and calcium stearate, stearic acid and hydrogenated vegetable oils.

Disintegrants can be used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

Stabilizers can be used to inhibit or retard drug decomposition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

Methods of Using the BRCA1 Modulating Compounds and Pharmaceutical Formulations Thereof

In use, the BRCA1 modulating compounds and formulations thereof described herein can be administered to a subject or one or more cells within the subject. The compounds once administered can be circulated through the subject and/or the one or more cells and can modulate a BRCA1, such as a mutated BRCA1. In some aspects, the mutation is BRCA1_{5382insC} mutation. In some aspects, the mutation can be detected in a cancer cell. In some aspects, the mutation is detected in a non-cancer cell. In some embodiments, one or more cells of the subject can also be administered a compound to increase the oxidative stress of one or more cells in the subject, such as tumor cells. In some embodiments, the compound that increases the oxidative stress of one or more cells in the subject can be hydrogen peroxide, chemotherapeutic agents, and/or endogenous cellular stress mimetics. Oxidative stress can be imparted to create cellular stress from a variety of endogenous and/or exogenous sources. For example and in addition to those already discussed, the body's own metabolic processing of endogenous and/or exogenous compounds, including but not limited to hormones and chemotherapeutic agents. Other compounds will be appreciated by those of ordinary skill in the art in view of this discussion.

In some embodiments, an amount of BRCA1 modulating compounds and/or formulations thereof can be administered to a subject. The subject can have or is suspected of having a cancer or a tumor. The cancer can be a breast cancer. The cancer can be an ovarian cancer. The cancer can be a cancer associated with and/or caused at least in part by a BRCA1 mutation. The cancer can be a cancer associated with and/or caused at least in part by a BRCA1_{5382insC} mutation. The amount can be an amount sufficient to reduce the amount of ubiquitin on BRCA1 or mutated BRCA1 in a cell. The amount can be an amount sufficient, with or without a compound to induce oxidative stress, to increase ubiquitination of BRCA1 and/or other proteins in a cell, such as a cancer cell, and/or kill a cancer cell.

The BRCA1 modulating compound or formulation thereof described herein can be co-administered or be a co-therapy with another active agent or ingredient that can be included in the formulation or provided in a dosage form separate from the BRCA1 modulating compound or formulation thereof or formulation thereof. In some embodiments, the co-therapy can be a compound that can increase the oxidative stress of a subject or a population of cells within the subject.

The amount of the BRCA1 modulating compound or formulation thereof can range from about 0.1 μg/kg to up to about 1000 mg/kg or more, depending on the factors mentioned elsewhere herein. In certain embodiments, the amount can range from 0.1 μg/kg up to about 500 mg/kg, or 1 μg/kg up to about 500 mg/kg, 5 μg/kg up to about 500 mg/kg, 0.1 μg/kg up to about 100 mg/kg, or 1 μg/kg up to about 100 mg/kg, 5 μg/kg up to about 100 mg/kg.

Administration of the BRCA1 modulating compound or formulation thereof can be systemic or localized. The BRCA1 modulating compound or formulation thereof can be administered to the subject in need thereof one or more times per hour or day. In embodiments, the BRCA1 modulating compound or formulation thereof can be administered once daily. In other embodiments, the BRCA1 modulating compound or formulation thereof can be administered can be administered 2, 3, 4, 5, 6, 7, 8, 9, 10, 11, 12, or more times daily. In some embodiments, when administered, an effective amount of the BRCA1 modulating compound or formulation thereof can be administered to the subject in need thereof. The BRCA1 modulating compound or formulation thereof can be administered one or more times per week. In some embodiments, BRCA1 modulating compound or formulation thereof can be administered 1, 2, 3, 4, 5, 6 or 7 days per week. In some embodiments, the BRCA1 modulating compound or formulation thereof can be administered 1, 2, 3, 4, 5, 6, 7, 8, 9, 10, 11, 12, or more times per month. In some embodiments, the BRCA1 modulating compound or formulation thereof can be administered 1, 2, 3, 4, 5, 6, 7, 8, 9, 10, 11, 12, 13, 14, 15, 16, 17, 18, or more time per year.

In some embodiments, the BRCA1 modulating compound or formulation thereof can be administered in a dosage form. The amount or effective amount of the BRCA1 modulating compound or formulation thereof can be divided into multiple dosage forms. For example, the effective amount can be split into two dosage forms and the one dosage forms can be administered, for example, in the morning, and the second dosage form can be administered in the evening. Although the effective amount can be given over two or more doses, in one day, the subject can receive the effective amount when the total amount administered across all the doses is considered. The dosages can range from about 0.1 μg/kg to up to about 1000 mg/kg or more, depending on the factors mentioned above. In certain embodiments, the dosage can range from 0.1 μg/kg up to about 500 mg/kg, or 1 μg/kg up to about 500 mg/kg, 5 μg/kg up to about 500 mg/kg, 0.1 μg/kg up to about 100 mg/kg, or 1 μg/kg up to about 100 mg/kg, 5 μg/kg up to about 100 mg/kg.

In some embodiments, the method can include administering a BRCA1 modulating compound or formulation thereof to a subject in need thereof. In some aspects, the method can include detecting BRCA1 mutation in one or more cells of the subject. In some aspects, the BRCA1 mutation is the BRCA1_{5382insC} mutation.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

Introduction.

The breast cancer susceptibility protein (BRCA1) coordinates DNA repair through a variety of mechanisms designed to protect genetic material (1-5). BRCA1 performs these duties in association with its binding partner the BRCA1-associated Ring Domain protein (BARD1). In the nucleus, the BRCA1-BARD1 heterodimer interacts with other repair proteins at DNA lesions to function as an E3-ubiquitin ligase (6-8). Through a series of exquisitely controlled steps BRCA1-BARD1 facilitates the transfer of ubiquitin moieties to a variety of nuclear protein substrates (9). These ubiquitin adducts direct their bound substrates toward different fates, one of which is involves correcting DNA damage.

Base-excision repair (BER) is a process that corrects non-helix distorting damage to DNA caused by conditions such as oxidation. BRCA1 plays an essential role in helping cells deal with oxidative conditions by triggering BER pathways through ubiquitin signaling (10, 11). Indeed, cells harboring inherited mutations in the BRCA1 gene cannot adequately deal with increased levels of reactive oxygen species (ROS) arising from estrogen metabolism. These inadequacies lead to functional deficiencies in BER, an accumulation of DNA insults, and widespread genomic instability—a known hallmark of cancer induction (12-15). Ultimately, the weakened state of mutated BRCA1 in oxidative environments supports disease progression.

It was recently reported that a prevalent clinical mutation, BRCA1_{5382insC}, influences the manner in which BRCA1 itself is modified in cancer cells (16). The main type of modification identified on BRCA1_{5382insC} was K48-linked ubiquitin chains. In the nucleus of cancer cells, higher levels of ubiquitination correlated with lower levels of active BRCA1_{5382insC} and changes in its biochemical properties. An ubiquitin attachment site on BRCA1 in ovarian cancer cells has been identified. This site, termed the “degron” sequence, is proximal to the BRCA1 N-terminal RING domain (17). While many studies have connected modifications in BRCA1 to changes in cellular activity, what remains missing from these analyses is the face of BRCA1.

Increased knowledge of BRCA1's three-dimensional (3D) structure can provide new insights for rational drug design and precision medicine. Structural information is currently available for the BRCA1-BARD1 RING domains (18) as well as the BRCA1 C-terminal (BRCT) region (19). However, the molecular architecture of full-length BRCA1 has not been determined. However, it is not understood how clinical mutations in BRCA1 affect its structure-function relationship and if mutated forms of BRCA1 be rescued or restored to normal. Similarly, the manner in which BRCA1 adapts to environmental changes or stressful conditions is poorly understood at the molecular level.

As described, inter alia, in this Example, the combination of biochemical and structural biology tools were used to study BRCA1 and some of its mutations, particularly those related to its involvement in cancer development and progression. Specifically, single particle electron microscopy (EM) was used to investigate differences among BRCA1-BARD1 structures derived from human breast cancer cells. A high degree of similarity between wild type and mutated assemblies under normal growth conditions was observed. During chemically-induced oxidative stress, a lysine-rich “hot spot” region on mutated BRCA1_{5382insC} was observed to be readily ubiquitinated. Structural evidence suggests this hot spot encompasses the documented degron sequence of BRCA1. Excessive ubiquitination in this area of the protein can be related to the previously noted decreases in BRCA1_{5382insC} activity (16, 17). As is described herein, the enzymatic removal of ubiquitin moieties in the hot spot region restored the overall structure of mutated BRCA1 assemblies. Taken together, this Example can describe and demonstrate, inter alia, a new lens to view BRCA1 along with the opportunity to transform its molecular properties.

Materials and Methods

Authentication of cells, cell culture, and protein enrichment. Breast cancer cells (HCC70 and HCC1937 lines) used in this study were purchased from the American Type Culture Collection (ATCC) and independently characterized by ATCC as being triple negative primary ductal carcinoma cells. For all experiments, cells were promptly used within 6 months of resuscitation. Cells were cultured in RPMI-1640 (Mediatech) supplemented with 10% fetal bovine serum (ATCC) and 0.5× penicillin-streptomycin (Thermo Fisher) at 37° C. and 5% CO₂ environment. BRCA1-BARD1 complexes were enriched as previously described (25). Briefly, about 1,000,000 cells were collected using Trypsin-EDTA (Thermo Fisher) followed by centrifugation (500×g, 5 minutes). For experiments involving hydrogen peroxide (H₂O₂), cells were collected with cell scrapers after treated with 1 mM of H₂O₂ (Sigma Aldrich) for 40 minutes at 37° C. and 5% CO₂. Sub-cellular fractions (cytoplasmic and nuclear) were separated using the NE-PER kit (Thermo Scientific). The soluble nuclear material was incubated with Ni-NTA agarose beads (Qiagen) and incubated with rotation for 1 hour at 4° C. The beads were washed with five bed volumes of 20 mM HEPES buffer (pH 7.2, 140 mM NaCl, 2 mM CaCl₂, 2 mM MgCl₂ and 5 mM imidazole). Phosphorylated BRCA1-BARD1 naturally bound to the Ni-NTA column matrix and was eluted in the same HEPES buffer, supplemented with 150 mM imidazole. Protein concentrations were determined using the standard Pierce Bradford assay (Thermo Scientific).

Coomassie Blue Staining and Immunoblot Analysis. Protein fractions were analyzed by SDS-PAGE followed by either Coomassie blue staining or western blotting. Proteins were separated on 3-8% Tris-Acetate NuPAGE mini gels (Thermo Fisher) and Stained by SimplyBlue SafeStain solutions (Invitrogen) for 60 minutes. Gels were washed with the deionized water for 60 minutes then 3% NaCl for an additional 2 hours—overnight to achieve maximum sensitivity. Western blots were performed as described previously (16). The following primary antibodies were used in our analysis: BRCA1-C20 (SCBT; sc-642; alpha-BRCT), BRCA1-Ab1 (Calbiochem; OP92; alpha-RING), BRCA1-A8X9F (Cell Signaling; #14823; alpha-RING), BARD1 (SCBT; sc-11438), ubiquitin-pAb (Enzo, ADI-SPA-200), RAD51 (SCBT, sc-8349), and beta-Actin (Sigma Aldrich; A5441).

Co-Immunoprecipitation (IP) Analysis. To detect protein-protein interactions, co-IP experiments were performed on isolated BRCA1-BARD1 protein fractions using previously described procedures (16). Antibodies used for IP experiments included BRCA1-C20 (5 μg, SCBT; sc-642) and BARD1 (5 μg, SCBT; sc-11438).

Deubiquitinase Assay. BRCA1_{5382insC}-BARD1 protein fractions isolated from H₂O₂-treated cells were used for deubiquitinase assays. Reaction mixture (200 μl total volume) contained 180 μl of the protein fraction and 20 μl of 10× USP2 catalytic domain (500 nm final concentration; UbiCREST, K-400; Boston Biochem). Control mixtures were prepared with 180 μl of the protein fraction and 20 μl of 1× DUB reaction buffer (UbiCREST, K-400; Boston Biochem) lacking the enzyme. Both reaction and control tubes were incubated at 37° C. water bath. After 30 minutes, samples were directly analyzed by either EM imaging or SDS-PAGE and western blot analysis. Prior to EM specimen preparation, free ubiquitin was removed from the samples using a Pierce Concentrator (100K MWCO, 0.5 ml, Thermo Scientific).

EM specimen preparation and imaging. Samples of isolated wild type, mutated, and modified BRCA1-BARD1 assemblies (0.02 mg/ml in 20 mM HEPES buffer pH 7.2, 150 mM NaCl, 10 mM CaCl₂, 10 mM MgCl₂) were applied to glow-discharged, continuous carbon support films on copper grids (Ted Pella) or to EM Affinity Grids (24, 38). Affinity grids were decorated with antibodies against the BRCA1 RING domain (EMD Millipore; MS110, AB1) or the BRCT domain (C-20) for labeling studies conducted on wild type assemblies. Protein complexes were tethered to the antibody-decorated grids by incubating Ni-NTA eluates for 2 minutes, followed by standard negatively staining procedures using 1% uranyl formate (39). Specimens were examined using a FEI Transmission Electron Microscope (TEM) (FEI Company) equipped with a LaBe filament and operating at 120 kV under low dose conditions (<5 electrons/Å₂). Images were recorded using an Eagle 2k HS CCD camera (FEI Company) with a pixel size of 30-μm at a magnification of about 68,000× for a final sampling of 4.4 Å/pixel.

Image processing. Image processing procedures are summarized schematically in Table S1. Individual particles were selected from the EM images using the SPIDER Software package (21). Selected particles were subjected to reference-free alignment routines implementing K-means classification to compute 2D class averages. Particles contained in the averages were grouped into image stacks and exported to the RELION software package (22). The RELION software package was used to refine and reconstruct the individual complexes employing an initial model of a sphere having a diameter of about 120 Å. The model was employed in the initial round of refinement. Later iterations were heavily dependent on the experimental data to refine the assigned angles by setting the regularization parameter to T=4. We followed standard reconstruction routines and employed a pixel size of 4.4 Å to produce 3D structures masked at about 120 Å. Equivalent contour levels were used to compare the EM maps among the various structures in the Chimera program (40). Threshold values for display are indicated in the EM map depositions and accommodated molecular volumes equivalent to about 320-about 350 kDa and 120 Å in diameter.

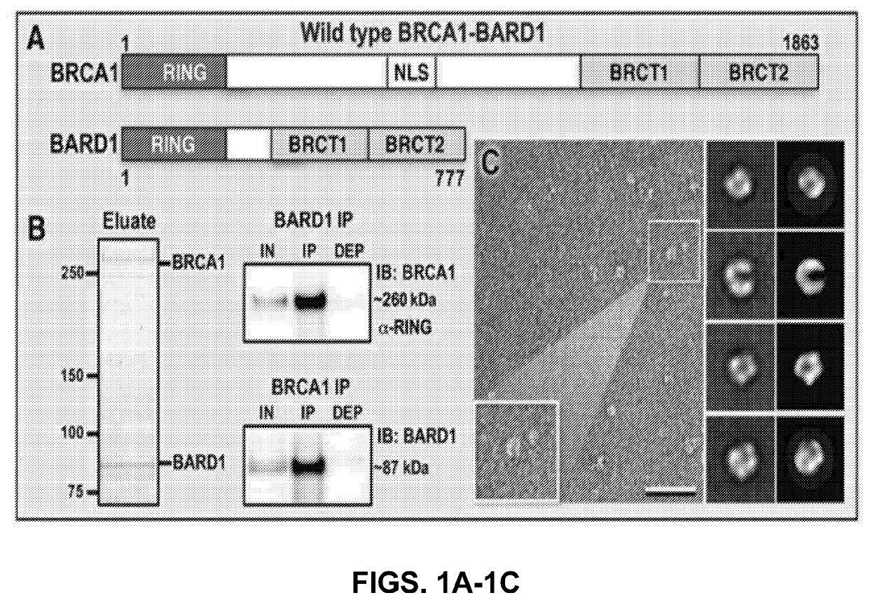

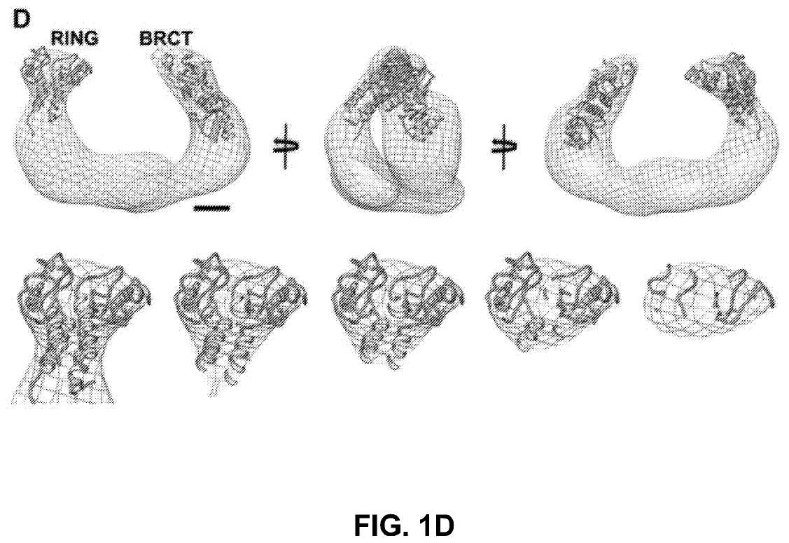

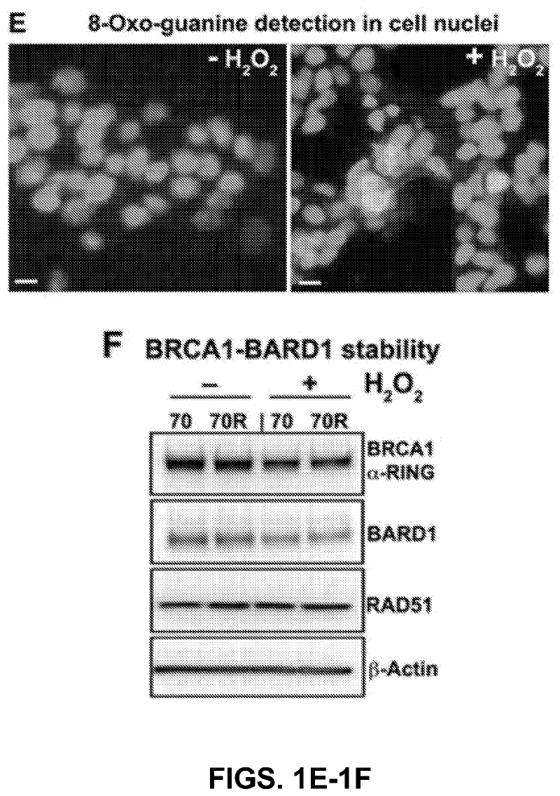

Particle heterogeneity for each sample was evaluated at the 2D and 3D classification steps. Class averages were calculated separately for each sample that included: 1) Wild type BRCA1-BARD1; 2) Mutated BRCA1-BARD1; 3) Mutated BRCA1-BARD1, H₂O₂-treated; 4) Mutated BRCA1-BARD1, USP2-treated. Particles in the 2D averages that displayed high contrast features and were sufficiently separated from other particles were used for reconstruction routines in RELION. This inspection procedure is standard practice in the EM field (22). At the level of 3D classification, RELION parameters were first implemented to output 3-5 classes from each image stack. For each sample, statistical values output from RELION following 25 iterations of refinement indicated the particle data could be combined into a single composite structure. Composite structures were subsequently calculated for each sample using the same input parameters. The final density maps, inter alia, are shown in FIGS. 1A-4D. The resolution of each map was determined by dividing the particle data for each reconstruction into two halves and calculating separate density maps. The FSC-0.5 criteria was used to determine the final resolution of each structure, then independently verified these values using the REMEASURE program (26). The final structure of wild type BRCA1-BARD1 (14.5 Å) contained 4008 particles. The structure of the mutated untreated BRCA1_{5382insC}-BARD1 (14.7 Å) contained 4222 particles, while the structure of the mutated H₂O₂-treated BRCA1_{5382insC}-BARD1 (15.6 Å) contained 4103 particles. The USP2-treated BRCA1_{5382insC}-BARD1 structure (15.4 Å) contained 4000 particles.

Difference maps. Difference densities between the H₂O₂-treated BRCA1_{5382insC}-BARD1 map and the untreated BRCA1_{5382insC}-BARD1 map were calculated. Maps were normalized and to a common density range and differences were generated using the publically available DIFFMAP executable. Difference densities in comparable regions at or above a 3σ-threshold were considered significant (28). A second difference map was calculated using the same procedures to visualize significant differences between the H₂O₂-treated BRCA1_{5382insC}-BARD1 map and the USP2-treated BRCA1_{5382insC}-BARD1 map.

Movie production. The Chimera software package (40) was used to produce each movie. Each structure was orientated similarly and a python file of the scene was exported. Following this routine, a Chimera command file was produced for each structure. These files contain a list of command line instructions that Chimera parses and applies to the scene. At the beginning of each movie a trio of labels are generated to indicate the location of the RING and BRCT domains, and the hot spot region. For cross-section views, camera slices were produced over a period of 110 frames. This procedure was reversed, as frame slices were replaced. Each structure was rotated about the x axis by 1 degree per frame for 90 frames. The slicing procedure was repeated, then again reversed. For rotated views, each structure was rotated about the y axis by 2 degrees 45 frames (90 degrees). The labels to indicate the hot spot region along with the RING and BRCT domains appear accordingly.

Results

Wild type BRCA1-BARD1 forms a stable clamp-like motif. To determine the 3D architecture of BRCA1-BARD1 natively formed in breast cancer cells (FIG. 1A), biochemical tools and single particle EM imaging technology were employed. Here, the focus was on visualizing differences between wild type and genetically mutated or modified BRCA1. Wild type BRCA1-BARD1 heterodimers (about 300 kDa) produced in the nucleus of primary ductal carcinoma cells (HCC70 line (20); ATCC) were enriched by incubating nuclear extracts with Nickel-Nitrilotriacetic acid (Ni-NTA)-coated agarose beads. Phosphorylated BRCA1-BARD1 heterodimers involved in DNA damage response naturally bound to the beads and eluted in early fractions. The phosphorylated form of BRCA1 migrated at about 260 kDa on SDS-PAGE analysis and BARD1 migrated at about 87 kDa (FIG. 1B).

To verify interactions between BRCA1 and BARD1 we performed co-immunoprecipitation (co-IP) experiments. Antibodies against BARD1 (Santa Cruz Biotechnology (SCBT)) or BRCA1 (C-20; SCBT) were decorated onto protein G-labeled magnetic beads and the protein fractions were incubated with the beads. The magnetically separated material was analyzed using western blot detection. BRCA1-BARD1 interactions were identified by probing the blots with antibodies against the BRCA1 RING domain or against BARD1 (FIG. 1B). After confirming protein associations, we examined the BRCA1-BARD1 complexes using single particle electron microscopy.

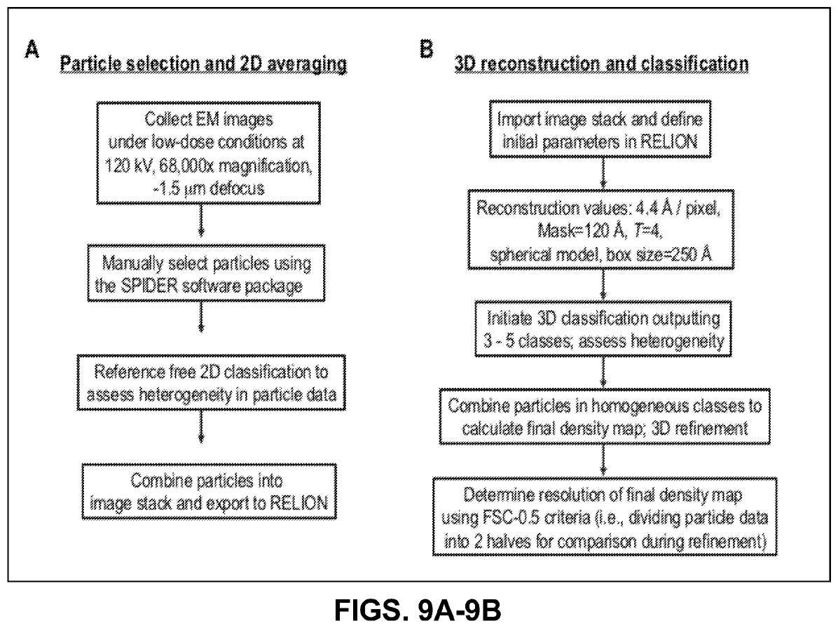

Low-dose images (<5 electrons/Å₂) were acquired for BRCA1-BARD1 specimens using a FEI Spirit Bio-Twin Transmission Electron Microscope (TEM) operating at 120 kV (FIG. 1C). Individual complexes were selected from the images using the SPIDER software package (21). Selected particles were subjected to standard reference-free alignment techniques also implemented in SPIDER. Class averages for wild type BRCA1-BARD1 showed clamp-like structures with a diameter of about 120 Å (FIG. 1C). The particles images were imported into the RELION software package (22) that was used to reconstruct and refine an EM density map (see Materials and Methods Section in this Example and FIGS. 9A-9B).

The 3D structure of wild type BRCA1-BARD1 confirmed a clamp-like motif that was ˜120 Å across its long axis and consistent with the class averages (FIG. 1D, movie S1). Two-dimensional projections of the 3D structure were in good agreement with the class averages (FIG. 1C). Examining the density map in various orientations provides a conformational snapshot of the heterodimer in solution. The general molecular architecture of the complex resembled another recently determined E3-ubiquitin ligase structure of comparable molecular mass to BRCA1 (23).

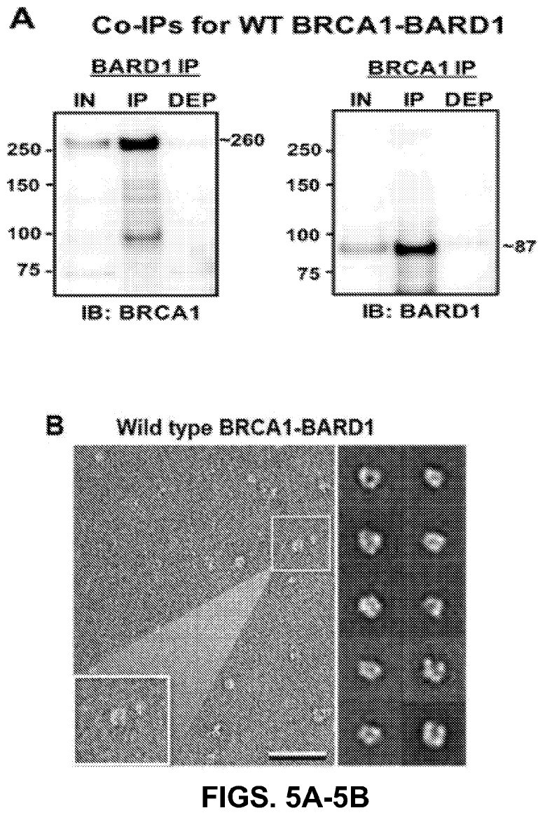

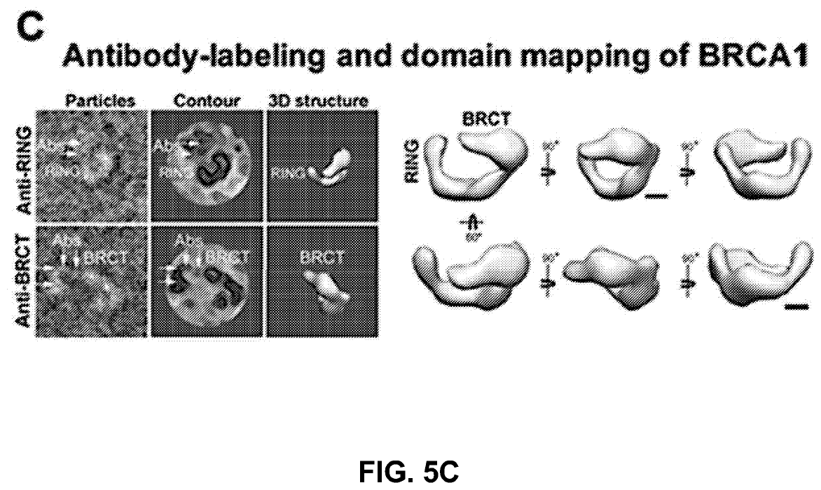

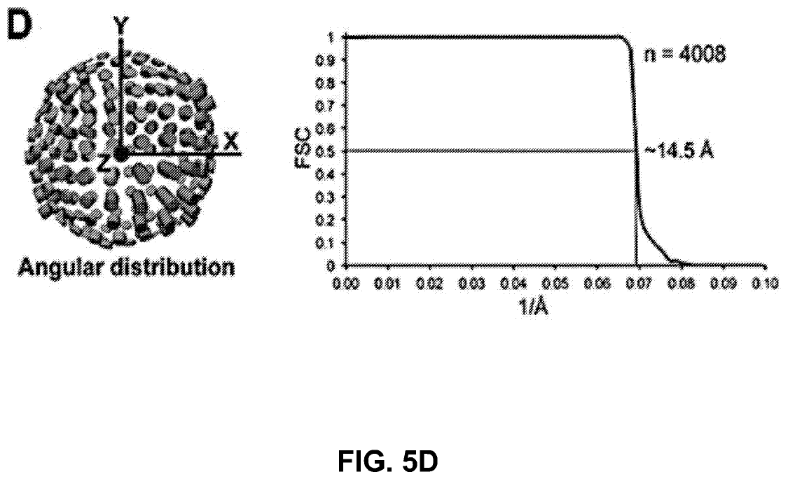

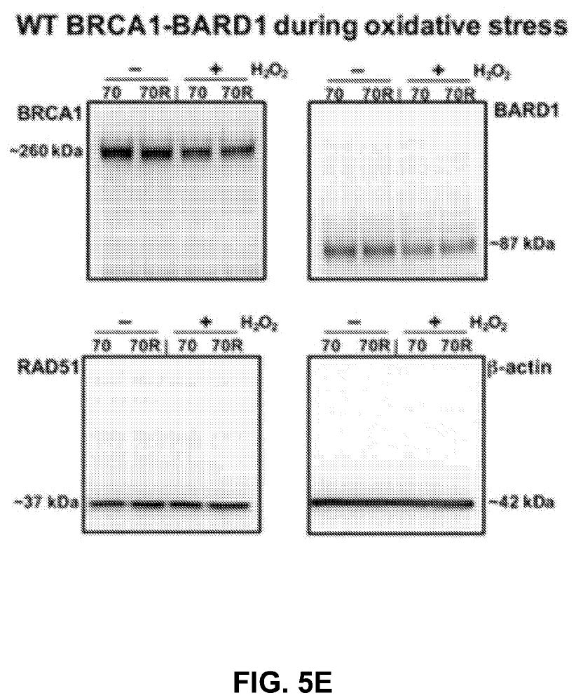

To distinguish the BRCA1 RING domain from the BRCT region, anEM Affinity Grids (24, 25) was used. Affinity Grids were separately decorated with antibodies against each component (FIGS. 5A-5E). Atomic models of the RING (pdbcode, 1JM7 (18)) and the BRCT ((pdbcode, 1JNX (19)) domains were placed in the density map according to positions defined by antibody-labeling results. The respective models could only fit in the density maps as indicated due to their unique features (FIG. 1D). The quality of the model fit is demonstrated in cross-sectional views shown in movie S1. The particles did not show limited orientations in their angular distribution and the structure was refined to 14.5 Å according to the 0.5-Fourier Shell Correlation (FSC) criteria in RELION (FIGS. 5A-5E). The resolution of the map was verified using the RMEASURE application (26). The calculated molecular volume of the density map accommodates one BRCA1-BARD1 dimer.

As expression levels and cellular stress can impact the functional response of BRCA1-BARD1 to DNA lesions, we tested for protein stability in breast cancer cells under stressful conditions. We induced oxidative stress by incubating cells with culture media containing 1 mM H₂O₂ for up to 60 minutes, as previously described (16). Fluorescence microscopy was used to detect antibodies against 8-Oxo-guanine (8-OxoG, SCBT) accumulation in genomic DNA. The formation of 8-OxoG is a direct marker for oxidative DNA damage in the nucleus.

Following a 40-minute incubation with H₂O₂, 8-OxoG signal (red fluorescence) increased in and around the nucleus of treated cells (blue fluorescence) (FIG. 1E). Control cells received culture media lacking H₂O₂ and showed no signal for 8-OxoG during the incubation period. Following 60 minutes of treatment, viability issues in treated cells limited measurements. As an additional control, we also included in our analysis HCC70 cells that experienced mild thermal stress prior to H₂O₂ treatment. These cells (HCC70-R) were primed to deal with cellular stress conditions and provided a model for oxidative resistance (16). Western blot comparisons of protein levels in treated cells showed that BRCA1 and BARD1 decreased modestly (about 10-20%) in replicate experiments. As an independent control, we also assessed nuclear RAD51 levels and found little to no change in protein quantities during treatment. Nuclear beta-Actin served as a loading control for western blot analyses. Overall, these results can suggest wild type BRCA1 and BARD1 levels were relatively stable in the nucleus during oxidative conditions and DNA damage response.

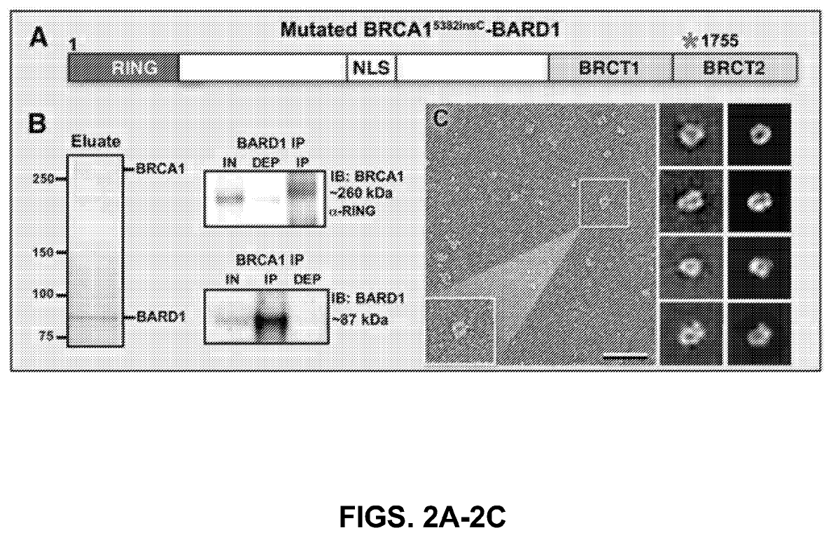

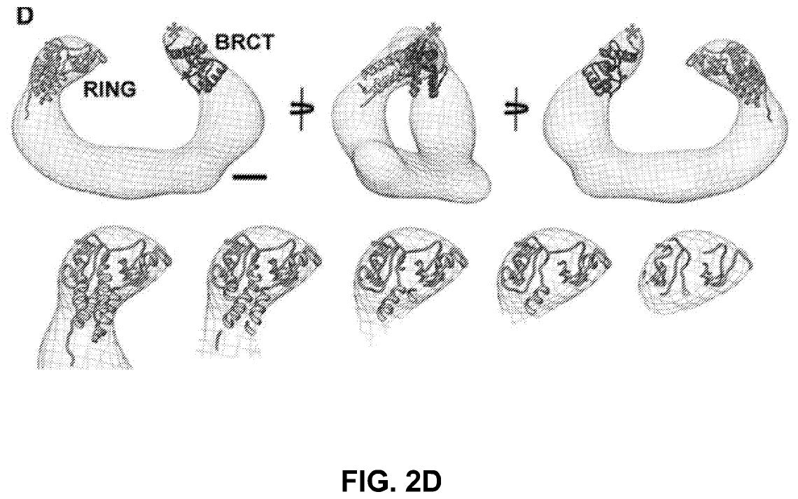

How does the BRCA1_{5382insC} clinical mutation affect protein structure? After gaining insight on wild type BRCA1-BARD1, we focused on learning more about the BRCA1_{5382insC} cancer-related mutation. Without being bound by theory, it was hypothesized that the mutated BRCA1_{5382insC} protein may adopt a slightly different architecture. A frame-shift mutation in the BRCA1_{5382insC} C-terminus occurs at residue S1755, resulting in about a10 kDa truncation (FIG. 2A). The same biochemical procedures were performed to isolate BRCA1_{5382insC}-BARD1 complexes from HCC1937 cells (ATCC) (27) that naturally express the mutated protein. According to SDS-PAGE analysis, BRCA1_{5382insC} migrated at about 260 kDa, similar to wild type BRCA1. Subtle differences in protein conformation may account for the higher than expected mobility of mutated BRCA1. BARD1 migrated at about 87 kDa and co-IP experiments confirmed BRCA1_{5382insC}-BARD1 interactions (FIG. 2B).

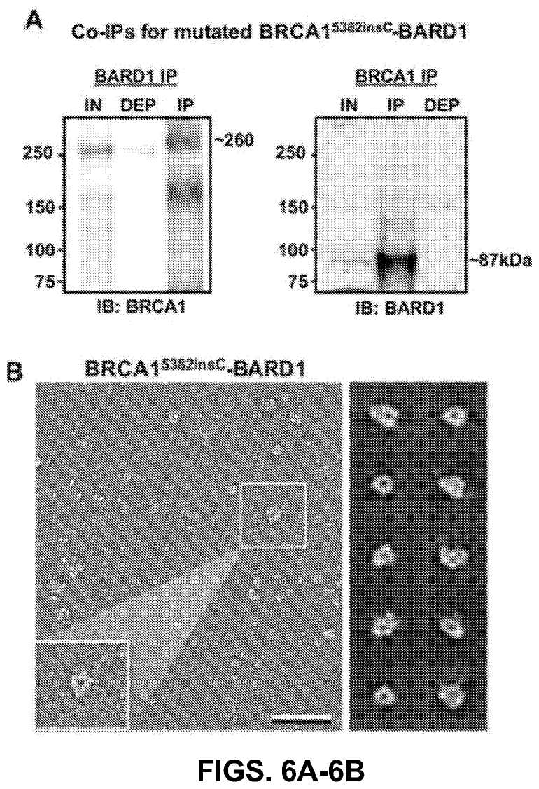

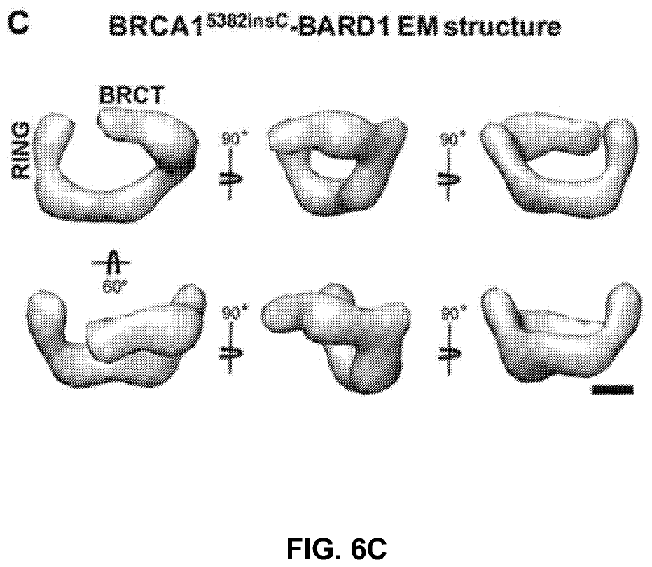

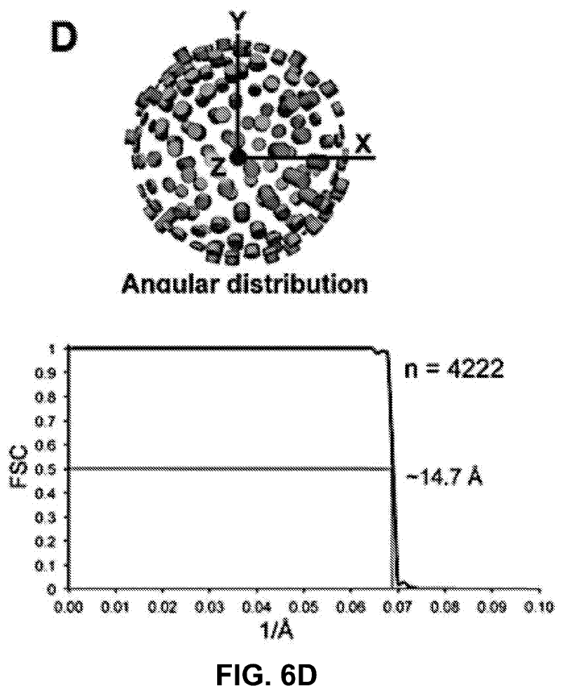

To determine the 3D structure of mutated BRCA1_{5382insC}-BARD1, we used the same imaging and computing procedures described for the wild type assemblies. Individual particles were selected from images and class averages were calculated using the SPIDER software package. The EM structure of the mutated BRCA1_{5382insC}-BARD1 complex revealed the same clamp-like motif seen in the wild type structure (FIGS. 2C and 2D). The dimeric RING domain fit well within the N-terminal density, and a homology model of the mutated BRCT domain (25) was placed in the C-terminal region of the map. The BRCT density was somewhat reduced in the mutated structure, which is expected considering the truncation (FIG. 2D). Cross-sections through the RING domain and EM density indicates the quality of the model fit from multiple views (movie S2). The particles did not show limited orientations in their angular distribution and the structure was refined to 14.7 Å according to the 0.5-FSC criteria in RELION (FIGS. 6A-6D). The resolution was verified using the RMEASURE application.

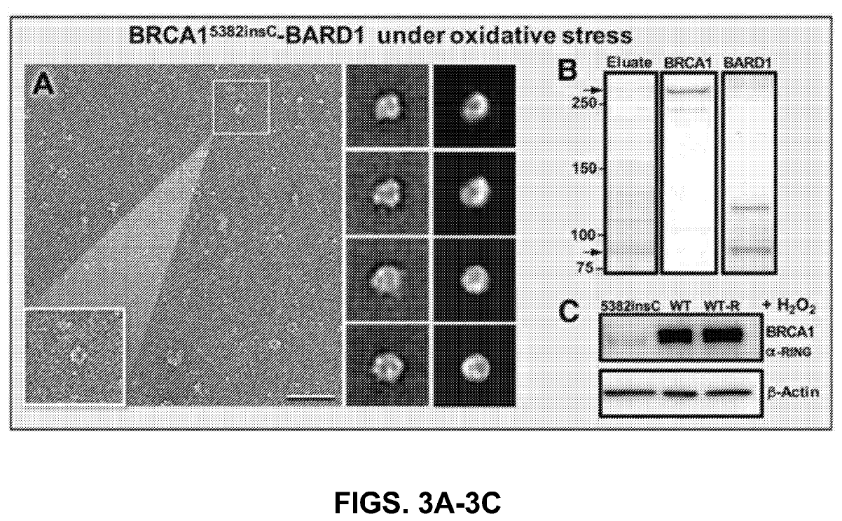

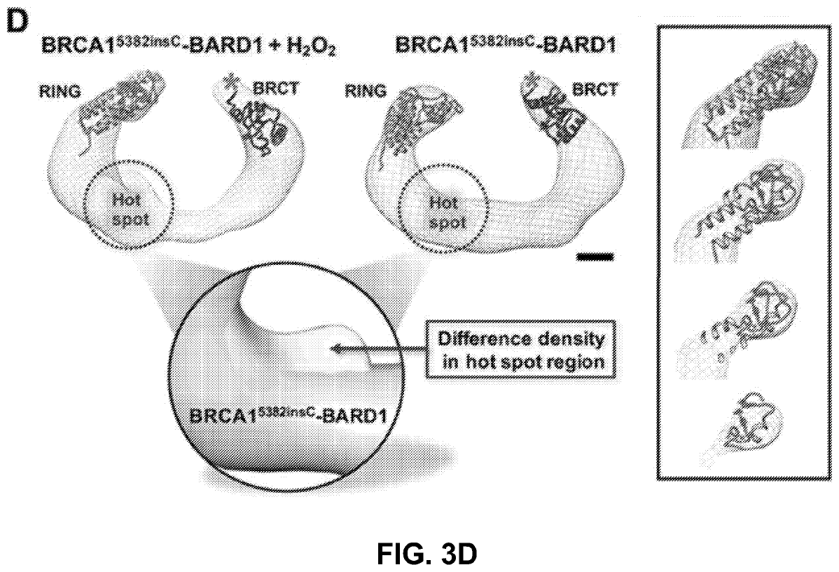

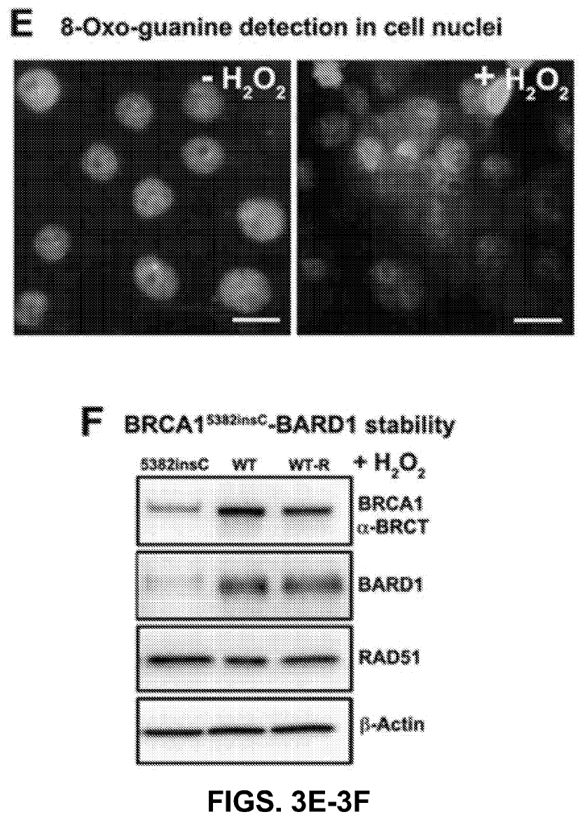

A “modification hot spot” identified on mutated BRCA1. Recent biochemical studies showed that mutated BRCA1 was highly susceptible to ubiquitination under oxidative stress conditions (16). Comparatively, wild type BRCA1 was not as susceptible to this effect. To understand the structural consequences of oxidative stress on mutated BRCA1, an EM analysis was performed on protein assemblies isolated from HCC1937 cells treated with H₂O₂ (FIG. 3A). Images and class averages showed a clamp-like conformation and particle dimensions were generally conserved in the treated BRCA1_{5382insC}-BARD1 complexes. One difference noted in the treated assemblies was a more compact shape than the untreated structures (FIG. 3A).

Taking a closer look at the protein components isolated from H₂O₂-treated cells, we found differences in SDS-PAGE and western blot analysis. Mutated BRCA1_{5382insC} migrated at about 270 kDa following H₂O₂ treatment while BARD1 migrated primarily at about 87 kDa (FIG. 3B). A higher molecular weight form of BARD1 was detected (about 120 kDa), but it did not associate with BRCA1. The nuclear material of treated cells expressing BRCA1_{5382insC} or wild type BRCA1 from two different sources (HCC70 and HCC70-R cells) was further examined. Western blots revealed a major decline in the detection of BRCA1_{5382insC} using antibodies against the RING domain (FIG. 3C). Without being bound by theory, this result may be due to decreased protein levels or limited accessibility near the BRCA1_{5382insC} RING epitope.

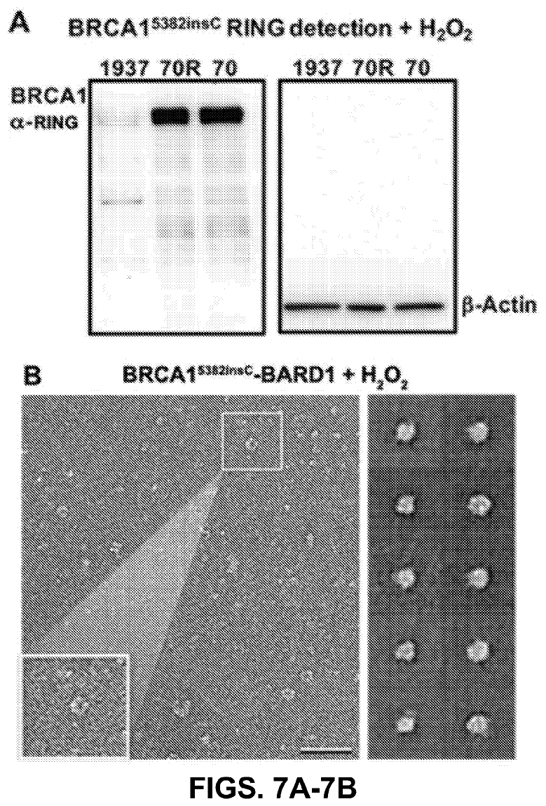

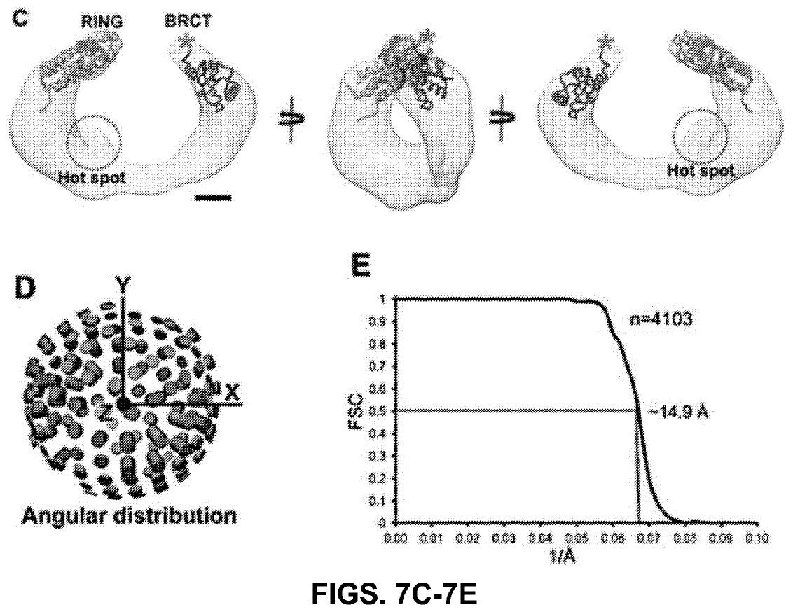

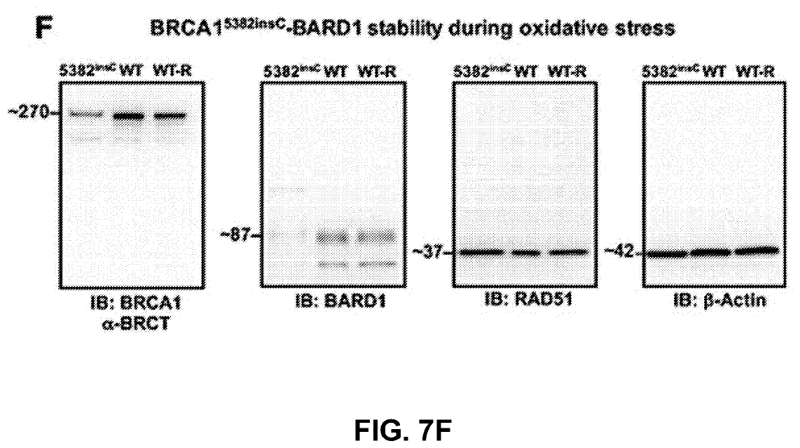

An EM density map was calculated for the BRCA1_{5382insC}-BARD1 complex isolated from H₂O₂-treated cells. The same imaging and computing methods were implemented in the SPIDER and RELION software packages. Models for the dimeric RING domain and mutated BRCT region fit well within the density as illustrated in FIGS. 3D, 7A-7F, and movie S3. Similar to the structures of wild type and untreated complexes, particle orientations were not limited and the map was refined to 15.6 Å according to the 0.5-FSC criteria determined in RELION and RMEASURE (FIG. S3). The BRCA1_{5382insC}-BARD1 structure produced under oxidative stress conditions was more compact and had extra density adjacent to the RING domain (FIG. 3D).

To better understand these physical changes, we calculated a difference map between the H₂O₂-treated BRCA1_{5382insC}-BARD1 structure and the untreated BRCA1_{5382insC}-BARD1 structure. Difference densities at or above a 3σ-threshold are considered statistically significant (28) . Based on this criteria, conformational changes in the central portion of the structure and the BRCT regions were smaller in comparison to differences near the RING domain, but were visibly present. A significant difference in the region proximal to the RING domain was observed, which is referred to as a modification “hot spot” (FIGS. 3D, 7A-7F, movie S3). The volume of the difference peak in the hot spot area (FIG. 3D, yellow) can accommodate protein density of about 12 kDa, which is sufficient to contain at least one ubiquitin moiety. Previous studies identified this region on BRCA1 to contain a “degron sequence” (17) . This degron site is a known target for K48-ubiquitination that can lead to proteasomal degradation of the protein.

BRCA1 stability was tested in the nucleus of H₂O₂-treated cells. Cells were incubated with 1 mM H₂O₂ for up to 40 minutes and fluorescence microscopy was used to detect 8-OxoG formation in and around the nucleus of the cells. Untreated control cells expressing BRCA1_{5382insC} contained 8-OxoG foci, a signature of oxidative DNA damage, at the start of the experiment (FIG. 3E, red foci). The 8-OxoG signal in the untreated cells persisted throughout the experiments but did not increase. Treated cells accumulated greater levels of 8-OxoG during the 40-minute incubation. Without being bound by theory, these results suggested that cells expressing mutated BRCA1_{5382insC} were not well-equipped to deal with oxidative conditions. This finding is important as reactive oxygen species are produced during estrogen metabolism, giving rise to oxidated DNA lesions (10, 11). As cells expressing mutated BRCA1_{5382insC} had inherent 8-OxoG accumulation, unlike cells expressing wild type BRCA1, there may be differences in the wild type and mutated proteins that influence these processes.

To evaluate the biochemical differences between wild type BRCA1 and mutated BRCA1_{5382insC} under oxidative conditions, protein levels in nuclear extracts were assessed. Western blot analysis performed on replicate experiments demonstrated that BRCA1_{5382insC} and BARD1 levels were reduced in H₂O₂-treated cells. Wild type proteins from two different cell sources (HCC70 and HCC70-R lines) were rather immune to the H₂O₂ treatment. Polyclonal antibodies against the BRCT domain of BRCA1 were used for detection to ensure an adequate comparison of protein signal, considering the RING domain may be less accessible, according to results in FIG. 3C. BRCA1_{5382insC} migrated at about 270 kDa, slower than the phosphorylated form of wild type BRCA1 (FIG. 3F). As an independent control we evaluated RAD51 levels, which were stable in the nucleus during treatment. Without being bound by theory, the data can indicate that oxidative stress alters the structure and the function of BRCA1_{5382insC} in breast cancer cells. These observations were consistent with other studies on mutated BRCA1 that show its DNA repair function is reduced during stressful situations (10-14).

Modified BRCA1_{5382insC}-BARD1 is altered by deubiquitinase treatment. Biophysical evidence presented here shows, inter alia, that cellular stress changes the molecular properties of mutated BRCA1. The evidence includes, but is not limited to, 1) a shift in the mobility of BRCA1_{5382insC} in SDS-PAGE and western blot analysis; 2) limited accessibility of the BRCA1_{5382insC} RING domain; 3) extra density in the BRCA1_{5382insC}-BARD1 structure proximal to the RING domain. To further test if ubiquitination accounts for these changes, protein fractions from cells undergoing oxidative stress were evaluated and treated with the deubiquitinase (DUB) enzyme, ubiquitin-specific protease 2 (USP2). USP2 removes a variety of ubiquitin adducts from protein substrates, generating mono-ubiquitin (about 8 kDa) upon removal. Protein fractions of BRCA1_{5382insC}-BARD1 isolated from H₂O₂-treated cells were incubated with catalytically active USP2 (500 nM; Boston Biochem) in HEPES buffer (pH, 7.5) for 30 minutes at 37° C. Control fractions received HEPES buffer solution lacking USP2.

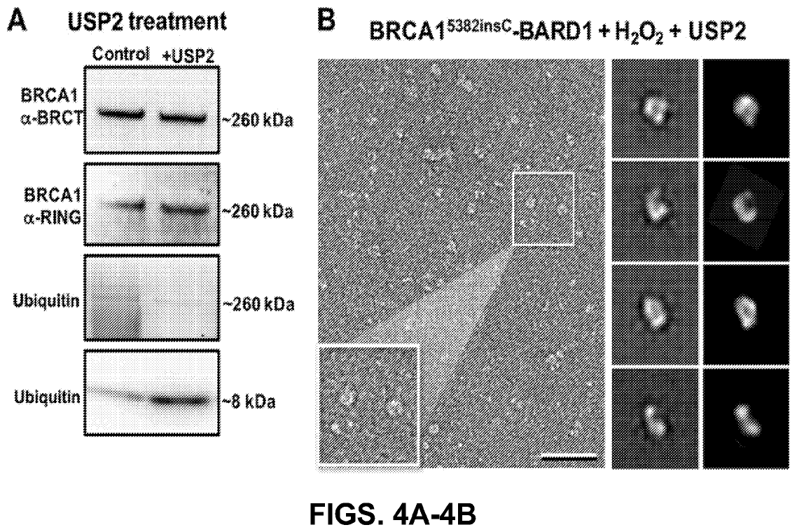

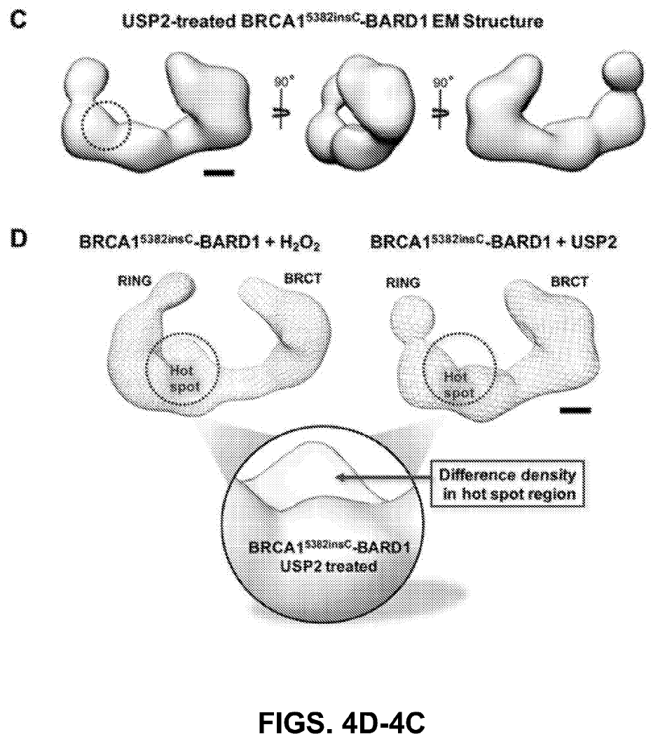

Biochemical analysis of USP2-treated fractions across replicate experiments showed a shift in BRCA1_{5382insC} mobility from about 270 kDa back to about 260 kDa. This band shift was consistent across multiple western blots probed with antibodies against the BRCT and RING domains of BRCA1 (FIG. 4A). Treated and control samples contained equal quantities of BRCA1_{5382insC} as indicated by equal detection of the BRCT domain. USP2-treated samples further showed a marked increase in the detection of the BRCA1_{5382insC} RING domain. Ubiquitinated products were also present in the control sample at about 270 kDa, and these bands were generally reduced in the USP2-treated fractions. Moreover, we detected a significant increase in mono-ubiquitin at the expected molecular weight of about 8 kDa in the treated fractions compared to the control samples (FIG. 4A).

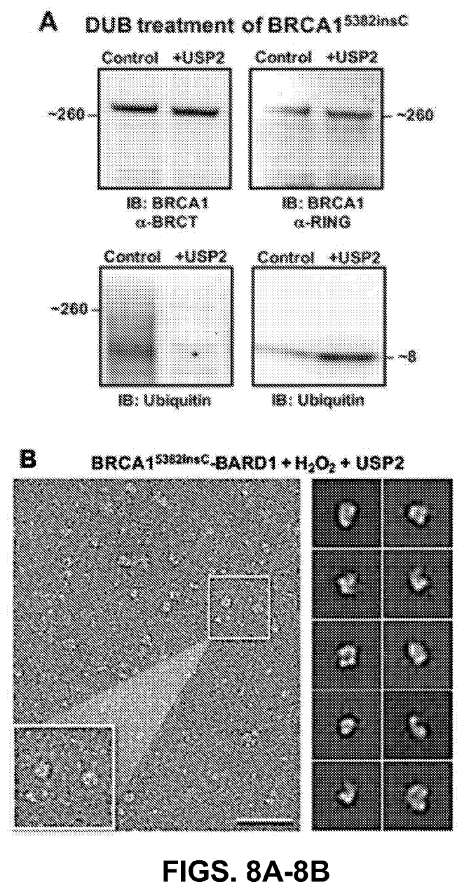

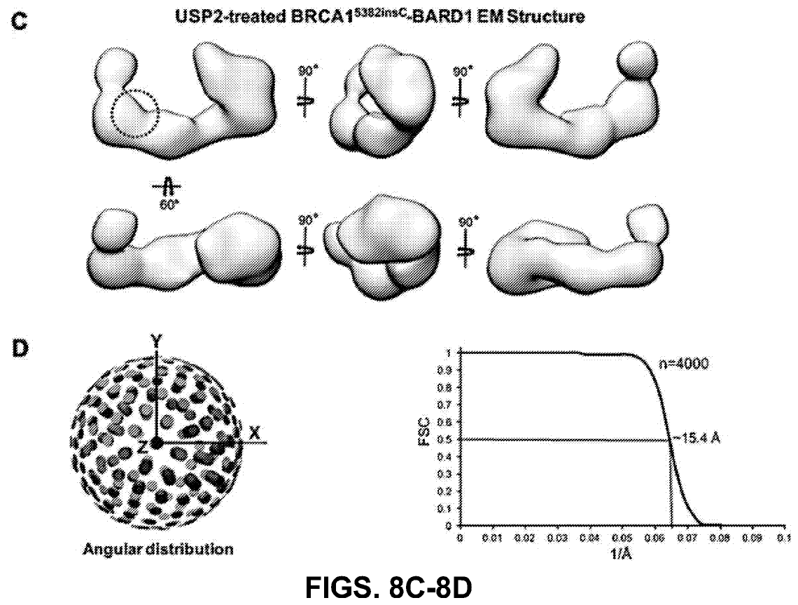

To understand these biochemical changes in the context of the 3D structure, we used single particle EM to examine deubiquitinated BRCA1_{5382insC}-BARD1. Images and class averages of USP2-treated BRCA1_{5382insC}-BARD1 (FIG. 4B) closely resembled the unmodified form of the heterodimer. The particles were less compact in nature and maintained the conserved clamp-shaped architecture. Again, particle orientations were not limited in the USP2-treated structure and the density map was refined to 15.4 Å using RELION and verified by RMEASURE (FIGS. 8A-8D).

The 3D structure of deubiquitinated BRCA1_{5382insC}-BARD1 lacked the extra density in the hot spot region adjacent to the RING domain (FIG. 4C, movie S4). To better visualize this change a difference map between comparable regions of the H₂O₂-treated BRCA1_{5382insC}-BARD1 structure and the USP2-treated BRCA1_{5382insC}-BARD1 structure were calculated (FIG. 4D). We implemented the same density threshold procedures described for FIGS. 3A-3F to highlight changes in the BRCA1 hot spot area. The resulting difference peak (FIG. 4D, yellow) shows the additional density that is present in the BRCA1_{5382insC}-BARD1 structure upon H₂O₂ treatment, but is lacking in the same region of the USP2-treated BRCA1_{5382insC}-BARD1 structure.

The lack of density in the deubiquitinated BRCA1_{5382insC}-BARD1 structure can indicated that ubiquitin moieties were distinctly removed from the BRCA1 hot spot area. There were also minor differences found in the molecular structure at the BRCT domain. However, these differences are likely due to enhanced flexibility or conformational variability in the mutated BRCT domain during oxidative stress and showed no signs of ubiquitin density. Overall, both the 2D averages and the 3D structure of USP2-treated BRCA1_{5382insC}-BARD1 can demonstrate that a modified, less functional form of BRCA1 can be modulated to restore its structural integrity.

Movie Summaries.

movie S1. Movie of the wild type BRCA1-BARD1 structure. Description: Movie showing slices through the wild type BRCA1-BARD1 structure from different views. The 3D reconstruction of the BRCA1-BARD1 heterodimer (cyan) was shown in different orientations to demonstrate the features of the density map. The RING domain (magenta; pdbcode, 1JM7 (18)) and BRCT domain (gray; pdbcode, 1JNX (19)) were fit in the EM envelope according to position information derived from antibody-labeling experiments. Cross-sections through the 3D reconstruction (cyan) of the BRCA1-BARD1 heterodimer are shown from alternative views. Traversing through the cross-sections, it was found that the atomic domains and the EM density disappeared simultaneously. This infers that the atomic models fully occupied each assigned density and indicates a suitable model fit in the map.

movie S2. Movie of the mutated BRCA1_{5382insC}-BARD1 structure. Description: Movie showing slices through the mutated BRCA1_{5382insC}-BARD1 structure from different views. The 3D reconstruction of the BRCA1_{5382insC}-BARD1 heterodimer (gray) is shown in different orientations to demonstrate the features of the density map and for comparison to the wild type structure. Atomic models used to interpret the EM map include the BRCA1-BARD1 RING domain (magenta; pdbcode, 1JM7 (18)) and a homology model for the BRCT domain (25) (red). Models positions are consistent with the wild type structure. Cross-sections through the 3D reconstruction (gray) show the quality of the model fit in the EM envelope.

movie S3. Movie of mutated BRCA1_{5382insC}-BARD1 isolated from H₂O₂-treated cells. Description: Movie showing slices through the 3D structure of BRCA1_{5382insC}-BARD1 isolated from H₂O₂-treated cells from different views. The 3D reconstruction of the BRCA1_{5382insC}-BARD1 heterodimer (yellow) is shown in different orientations to demonstrate the features of the density map and for comparison to the wild type and untreated BRCA1_{5382insC}-BARD1 structures. Unique features of the map include a bulky region in the “hot spot” area adjacent to the RING domain and the proximity of the two end regions that represent the RING and BRCT domains. The BRCA1-BARD1 RING domain (magenta; pdbcode, 1JM7(18)) and a homology model for the BRCT domain (25) (red) were used to interpret the reconstruction, similar to the density map derived from untreated cells. Cross-sections through the 3D reconstruction show the quality of the model fit in the EM envelope.

movie S4. Movie of mutated BRCA1_{5382insC}-BARD1 treated with USP2. Description: Movie of mutated BRCA1_{5382insC}-BARD1 treated with USP2. The 3D reconstruction of the USP2-treated BRCA1_{5382insC}-BARD1 heterodimer (green) is shown in a variety of orientations to demonstrate the features of the density map. The bulky density in the hot spot region, adjacent to the RING domain, is not present in the deubiquitinated structure. The absence of the excessive density in the hot spot area indicates that USP2-treated assemblies were biochemically modulated to renew their structural integrity.

Discussion

In summary, this Example describes information for full-length BRCA1-BARD1 isolated from human breast cancer cells. Structures formed under a variety of cellular conditions allowed us to directly compare wild type and mutated complexes. Each of the 3D structures adopted a conserved clamp-like motif with characteristic features found in other E3-ubiquitin ligases (29-31). Under normal growth conditions, there were subtle differences between wild type and mutated structures. For example, the BRCT domain of mutated BRCA1 was slightly truncated resulting in less density in this region of the reconstruction. In general, E3-ubiquitin ligases bring E2-conjugating enzymes in proximity to a substrate. The substrate binding region of BRCA1 resides in the BRCT domain. Hence, mutations that affect the structural properties of the BRCT can influence BRCA1's ability to transfer ubiquitin moieties to its substrates.

Under oxidative conditions, the mutated BRCA1-BARD1 structure showed attributes that were not present in the wild type or untreated structures. To better understand these changes, we performed additional biochemical experiments. These studies revealed BRCA1_{5382insC} migrated slower than wild type BRCA1 on western blot analysis, and the RING domain was less exposed in the mutated protein. Residues surrounding the BRCA1 RING domain can be ubiquitinated, phosphorylated, or sumoylated (32-34). Ubiquitin adducts were present in this region on mutated BRCA1 under oxidative conditions.

The general mechanisms by which ubiquitination is involved in DNA damage response is dynamic and complex. BRCA1 is one of many players that orchestrate protective measures against genotoxic insults. Other examples of ubiquitination playing a role in DNA repair involve regulatory events surrounding histone H2A modifications. USP51 was recently shown to deubiquinate H2A at Lys13 and Lys15 during double-stranded breaks resulting from ionizing radiation. This loss of ubiquitin signal on H2A prevented the proper recruitment of repair proteins to DNA lesions (35). Another recent study on H2A ubiqutination during UV-induced nuclear excision repair processes, pinpoints the biochemical players and steps involved in chromatin remodeling through the zuotin-related factor 1 (ZRF1) molecular switch (36). A complementary role for BRCA1 in ubiquitinating H2A at sites of DNA damage has also been well-established (37). However, BRCA1's ability to perform this important task is reduced as protein levels are diminished or its functional N- and C-terminal domains are compromised.

Recent biochemical studies demonstrated increases in K48-ubiquitination can lower functional levels of mutated BRCA1 in cellular assays (16). The structural analysis presented here further explains how mutated BRCA1 is affected by detrimental ubiquitination events. As irregularities in the BRCA1 structure were linked to functional deficiencies in cancer cells, it is intriguing to think the restoration of BRCA1's structural properties may improve its cellular activity. Ongoing efforts to test this idea are promising but fall outside the scope of the current report.

Overall, this Example can provide a unique outlook on the structure-function relationship of BRCA1 that is currently missing in the field. Deficiencies in mutated BRCA1 have been observed to relate to unwarranted ubiquitination in cells experiencing oxidative stress. Cells deficient in BRCA1 activity tend to accumulate DNA insults that provide a tipping point towards cancer induction (2, 14). Counter to this, this Example can demonstrate that detrimental changes to mutated BRCA1 can be biochemically tempered to renew its structural integrity.

### References for Example 1

1. P. L. Welcsh, K. N. Owens, M. C. King, Insights into the functions of BRCA1 and BRCA2. Trends Genet 16, 69-74 (2000).

2. S. Rowell, B. Newman, J. Boyd, M. C. King. Inherited predisposition to breast and ovarian cancer. Am J Hum Genet 55(5), 861-865 (1994).

3. E. M. Rosen, S. Fan, Y. Ma, BRCA1 regulation of transcription. Cancer Lett 236, 175-185 (2006).

4. D. J. Toft, V. L. Cryns, Minireview: Basal-like breast cancer: from molecular profiles to targeted therapies. Mol Endocrinol 25, 199-211 (2011).

5. C. M. Perou, Molecular stratification of triple-negative breast cancers. Oncologist 15 Suppl 5, 39-48 (2010).

6. P. Schwertman, S. Bekker-Jensen, and N. Mailand, Regulation of DNA double-strand break repair by ubiquitin and ubiquitin-like modifiers. Nat Rev Mol Cell Bio 17, 379-394 (2016).

7. W. Wu, A. Koike, T. Takeshita, T. Ohta. The ubiquitin E3 ligase activity of BRCA1 and its biological functions. Cell Div 3, 1-10 (2008).

8. R. Baer, T. Ludwig. The BRCA1/BARD1 heterodimer, a tumor suppressor complex with ubiquitin E3 ligase activity. Curr Opin Gene Dev 12, 86-91 (2002).

9. P. S. Brzovic, et al. Binding and recognition in the assembly of an active BRCA1/BARD1 ubiquitin-ligase complex. Proc Nat Acad Sci U.S.A. 10, 5646-5651 (2003).

10. I. Bae et al., BRCA1 induces antioxidant gene expression and resistance to oxidative stress. Cancer Res 64, 7893-7909 (2004).

11. F. Le Page et al., BRCA1 and BRCA2 are necessary for the transcription-coupled repair of the oxidative 8-oxoguanine lesion in human cells. Cancer Res 60, 5548-5552 (2000).

12. E. Alli, V. B. Sharma, P. Sunderesakumar, J. M. Ford, Defective repair of oxidative dna damage in triple-negative breast cancer confers sensitivity to inhibition of poly(ADP-ribose) polymerase. Cancer Res 69, 3589-3596 (2009).

13 E. Alli, J. M. Ford, BRCA1 : Beyond double-strand break repair. DNA Repair (Amst) 32, 165-171 (2015).

14. U. E. Martinez-Outschoorn et al., BRCA1 mutations drive oxidative stress and glycolysis in the tumor microenvironment: implications for breast cancer prevention with antioxidant therapies. Cell Cycle 11, 4402-4413 (2012).

15. D. Hanahan, R. A. Weinberg, Hallmarks of cancer: the next generation. Cell 144, 646-674 (2011).

16. B. L. Gilmore et al., Molecular Analysis of BRCA1 in Human Breast Cancer Cells Under Oxidative Stress. Sci Rep 7, 43435 (2017).

17. Y. Lu et al., Ubiquitination and proteasome-mediated degradation of BRCA1 and BARD1 during steroidogenesis in human ovarian granulosa cells. Mol Endocrinol 21, 651-663 (2007).

18. P. S. Brzovic, P. Rajagopal, D. W. Hoyt, M. C. King, R. E. Klevit, Structure of a BRCA1-BARD1 heterodimeric RING-RING complex. Nat Struct Biol 8, 833-837 (2001).

19. R. S. Williams, R. Green, J. N. Glover, Crystal structure of the BRCT repeat region from the breast cancer-associated protein BRCA1. Nat Struct Biol 8, 838-842 (2001).

20. A. F. Gazdar et al., Characterization of paired tumor and non-tumor cell lines established from patients with breast cancer. Int J Cancer 78, 766-774 (1998).

21. J. Frank et al., SPIDER and WEB: processing and visualization of images in 3D electron microscopy and related fields. J Struct Biol 116, 190-199 (1996).

22. S. H. Scheres, A Bayesian view on cryo-EM structure determination. J Mol Biol 415, 406-418 (2012).

23. D. Lyumkis et al., Single-particle EM reveals extensive conformational variability of the Ltn1 E3 ligase. Proc Natl Acad Sci USA 110, 1702-1707 (2013).

24. D. F. Kelly, D. Dukovski, T. Walz, Strategy for the use of affinity grids to prepare non-His-tagged macromolecular complexes for single-particle electron microscopy. J Mol Biol 400, 675-681 (2010).

25. B. L. Gilmore et al., A Molecular Toolkit to Visualize Native Protein Assemblies in the Context of Human Disease. Sci Rep 5, 14440 (2015).

26. D. Sousa, N. Grigorieff, Ab initio resolution measurement for single particle structures. J Struct Biol 157, 201-210 (2007).

27. G. E. Tomlinson et al., Characterization of a breast cancer cell line derived from a germ-line BRCA1 mutation carrier. Cancer Res 58, 3237-3242 (1998).

28. J. Frank, Three-Dimensional electron microscopy of macromolecular assemblies. Oxford Univ Press, Inc. New York (2006).

29. G. Nalepa, M. Rolfe, J. W. Harper, Drug discovery in the ubiquitin-proteasome system. Nat Rev Drug Discov 5, 596-613 (2006).

30. C. E. Berndsen, C. Wolberger, New insights into ubiquitin E3 ligase mechanism. Nat Struct Mol Biol 21, 301-307 (2014).

31. A. M. Simons et al., BRCA1 DNA-binding activity is stimulated by BARD1. Cancer Res 66, 2012-2018 (2006).

32. A. D. Choudhury, H. Xu, R. Baer, Ubiquitination and proteasomal degradation of the BRCA1 tumor suppressor is regulated during cell cycle progression. J Biol Chem 279, 33909-33918 (2004).

33. C. X. Deng, BRCA1: cell cycle checkpoint, genetic instability, DNA damage response and cancer evolution. Nucleic Acids Res 34, 1416-1426 (2006).

34. J. R. Morris et al., The SUMO modification pathway is involved in the BRCA1 response to genotoxic stress. Nature 462, 886-890 (2009).

35. Z. Wang et al., USP51 deubiquitylates H2AK13,15ub and regulates DNA damage response. Genes Dev 30, 946-959 (2016).

36. E. Gracheva et al., ZRF1 mediates remodeling of E3 ligases at DNA lesion sites during nucleotide excision repair. J Cell Biol 213, 185-200 (2016).

37. R. Kalb, D. L. Mallery, C. Larkin, J. T. Huang, K. Hiom, BRCA1 is a histone-H2A-specific ubiquitin ligase. Cell Rep 8, 999-1005 (2014).

38. C. E. Winton et al., A microchip platform for structural oncology applications. NPJ Breast Cancer 2, (2016).

39. M. Ohi, Y. Li, Y. Cheng, T. Walz, Negative staining and image classification—powerful tools in modern electron microscopy. Biol Proced Online 6, 23-34 (2004).

40. E. F. Pettersen et al. USCF Chimera—a visualization system for exploratory research and analysis. J Comput Chem 25, 1605-1612 (2004).

### Example 2

Introduction

Cells with mutations in the breast cancer type 1 susceptibility protein (BRCA1) have difficulty protecting their genetic material from repeated daily stressors (1, 2). These deficiencies create an unstable environment in the nucleus as DNA insults accumulate. Scientific evidence points to BRCA1's ubiquitin ligase activity as a key element in safeguarding the genome (3, 4). Consistent with this idea, disruptions in BRCA1's duties weaken genomic integrity and are linked to aggressive forms of cancer (5-7). In general, ubiquitin ligases act as warning systems inside cells. Ubiquitin signals are passed along from molecule to molecule coordinating protein turnover, nuclear escape, or repair processes. In this capacity, BRCA1 systematically adds ubiquitin tags to protein substrates, sounding the alarm that DNA has been compromised (8, 9). A recurring lapse in this cautionary procedure leaves the genome in a fragile state.

To learn more about the BRCA1 warning system and its role in cancer initiation, the structural properties of BRCA1 were investigated. It was recently reported the first structure of full-length BRCA1 interacting with its binding partner, the BRCA1-associated RING domain protein 1 (BARD1) (10). It that BRCA1's protective power in the nucleus was disrupted by changes in a conserved “hotspot” region of the mutated protein but not the wild-type protein. Under stressful cellular conditions, excessive ubiquitination in the hotspot area inappropriately targeted mutated BRCA1 for degradation (11). As shown in Example 1, ubiquitin can be removed from BRCA1 using deubiquitinase enzymes (DUBs) (10). In shortm DUB treatment successfully rescued the structure of mutated BRCA1.

In this Example, the functional restoration of a mutated BRCA1 and the therapeutic potential of altering a mutated BRCA1, inter alia, is described. This Example can demonstrate that the biochemical function of mutated BRCA1 can be greatly restored by DUB treatment. Restoring the actions of mutated BRCA1 was observed to elevate the cellular response of p53—a known substrate for BRCA1 (12, 13). Improving our knowledge of these interactions is important as mutations in the p53 tumor suppressor occur in about 50% of human tumors and about 80% of BRCA1-related cancers (14-16). Related to this finding, structural information for p53 assemblies naturally produced in cancer cells is also described. These assemblies were captured while poised on native DNA. Collectively, this Example can describe and demonstrate, inter alia, that the BRCA1 warning system can be exquisitely modulated while offering new strategies to mechanistically target cancer cells. Developing new tools to chemically nudge tumor suppressors in human cells holds great potential to improve disease management or preventative measures.

Materials and Methods.

Authentication of cells and cell culture conditions. Human breast cancer cells (HCC1937 line) were purchased from American Type Culture Collection (ATCC) and independently characterized by ATCC as triple-negative primary ductal carcinoma cells. Human glioblastoma multiforme (GBM) cells (U87MG, T98G, and A172 lines) and normal human astrocytes were kindly provided by Dr. Zhi Sheng at Virginia Tech Carilion Research Institute. For all experiments, cells were promptly used within 6 months of resuscitation. HCC1937 cells were cultured in RPMI 1640 (Mediatech) supplemented with 10% fetal bovine serum (ATCC) and 0.5× penicillin-streptomycin (Life Technologies). Glioma cells (U87MG, T98G, and A172 lines) were maintained in Dulbecco's Modified Eagle Medium (DMEM, Life Technologies) supplemented with 10% fetal bovine serum (Atlas Biologies), streptomycin (100 ug/ml, Gibco), and penicillin (100 IU/ml, Gibco). Normal human astrocytes were cultured in MCDB-131 Medium (Sigma) containing 3% fetal bovine serum (Peak Serum), 10× G-5 Supplement (Gibco), 100 μg/mL streptomycin and 100 IU/mL Penicillin (Gibco). Cells were free of contamination and cultured at 37° C. with 5% CO₂.

Protein isolation procedures. BRCA1-BARD1 complexes were isolated as previously described (10). Briefly, about 1 million cells were treated by adding hydrogen peroxide (1 mM H₂O₂, Sigma-Aldrich) to the culture media for various time points at 37° C. and 5% CO₂. Control cells received culture media lacking H₂O₂. Following the incubation period, cells were collected using cell scrapers. Cytoplasmic and nuclear fractions were separated using the NE-PER kit (Thermo Fisher Scientific). The soluble nuclear material was further incubated with Nickel-Nitrilotriacetic acid (Ni-NTA) agarose beads (Qiagen) with rotation for 1 hour at 4° C. The beads were washed with five bed volumes of 20 mM HEPES buffer (pH 7.2) containing 140 mM NaCl, 2 mM CaCl₂, 2 mM MgCl₂, and 5 mM imidazole). Phosphorylated BRCA1-BARD1 naturally bound to the Ni-NTA column matrix and was eluted in the same HEPES buffer, supplemented with 150 mM imidazole. Protein concentrations were determined using the standard Pierce Bradford assay (Thermo Fisher Scientific). The same cell lysis and Ni-NTA enrichment steps were used to isolate p53 from HCC1937 cells (p53_{R305}) and U87MG cells (p53_{WT}), except the protein assemblies were eluted with HEPES buffer (pH 7.2) supplemented with 60 mM imidazole.

Cell lysate preparations. For whole cell lysate preparations, HCC1937 cells were treated by adding 1 mM H₂O₂ to the culture media for 10-20 minutes, while incubating cells at 37° C. and 5% CO₂. Following each incubation time, cells were washed with 1× PBS (Sigma) to eliminate residual H₂O₂. The cells were then treated with 7 μM-9 μM ML364 (Axon Medchem) for up to 48 hours at 37° C. and 5% CO₂. Control cells were cultured in media lacking H₂O₂ and/or ML364 during the same time period. Cells were collected and washed with ice-cold 1× PBS once followed by centrifugation at 500×g for 5 minutes. Cell pellets were lysed by resuspending the pellets in buffer solution containing 20 mM HEPES (pH 6.8), 150 mM NaCl, 2.5 mM CaCl₂, 2.5 mM MgCl₂, 1 mM EDTA, 2% Nonidet-P40 (NP40), 1% Nadeoxycholate, 2× protease inhibitors (Sigma), 1× phosphatase inhibitors (Sigma), and 8% glycerol. The lysis mixture was incubated on ice for 30 minutes and centrifuged at 21,000×g for 15 minutes at 4° C. The supernatants were collected and protein concentrations were quantified using the standard Pierce Bradford assay.

Native Gel and Immunoblot Analysis.

For native gel analysis, protein fractions of purified p53 were combined with 4× NativePAGE sample buffer (Thermo Fisher Scientific). Native sample preps were separated using light blue cathode running buffer on a NativePAGE 3-12% Bis-Tris gel (1 hr at 150V then about 1 hour at 250V; Thermo Fisher Scientific). For coomassie staining, gels were fixed then stained with 0.02% Coomassie Brilliant Blue R-250 (Fisher Scientific). For immunoblotting, gels were placed in 2× NuPAGE transfer buffer (Thermo Fisher Scientific) for 10 minutes before transfer onto PVDF membranes (Millipore) in 1× NuPAGE transfer buffer at 25V for 1 hour at 4° C. The membrane was fixed in 8% acetic acid for 15 minutes followed by staining for 5 minutes in 0.1% coomassie R-250 in 50% methanol to visualize the NativeMark Unstained Protein Standards (Thermo Fisher Scientfic). The blot was destained in 50% methanol/10% acetic acid solution three times for 5 minutes, rinsed with several changes of purified water and allowed to air dry. The membrane was re-wet then blocked with 1% non-fat dry milk (NFDM) in TBS-T (0.05%) for 1 hour with gentle rocking. Anti-p53 (DO-1, Santa Cruz Biotechnology) primary antibody was diluted in blocking solution and incubated overnight at 4° C. Blots were washed three times with TBS-T (0.05%). Goat anti-rabbit secondary antibodies conjugated to horseradish peroxidase (Jackson ImmunoResearch) were incubated for 1 hour followed by additional washing. ECL Prime western blotting reagent (GE Healthcare) or West Femto (Thermo Fisher) was used for detection. A ChemiDoc MP (Bio-Rad) was used for imaging.

SDS-Page, immunoblotting, and densitometry measurements. Protein fractions were also analyzed by SDS-PAGE denaturing gels followed by staining with SimplyBlue SafeStain solution (Invitrogen) or Western blotting as previously described (10). For Western blot analysis, the following primary antibodies were used: BRCA1 (C-20; Santa Cruz Biotechnology, sc-642), ubiquitin-pAb (Enzo Life Sciences, ADI-SPA-200), p53 (DO-1; Santa Cruz Biotechnology, sc-126), K63-linkage specific polyubiquitin (D7A11; Cell Signaling, #5621) and p-actin (Sigma-Aldrich, A5441). Western blot quantification was performed using Image Labrm Software (Bio-Rad). The intensity of each band was selected using the volume tool. Local subtraction and linear regression methods were implemented to eliminate the local background values and to quantify the band intensities.

Deubiquitinase (DUB) assay. Purified BRCA1_{5382insC}-BARD1 fractions (0.1-0.2 mg/ml) were incubated with 500 nM USP2 catalytic domain (Boston Biochem) in a water bath at 37° C. USP2 is a general deubiquitinase enzyme (DUB). Control mixtures were prepared using the same protein fractions and concentrations, along with 1× DUB reaction buffer (Boston Biochem) that lacked USP2. These reaction mixtures were incubated in a water bath at 37° C. along-side the enzymatically-treated material. The inactive samples were incubated in parallel at 4° C. Following a 30-minute incubation period, samples were analyzed by EM imaging, SDS-PAGE, and Western blot analysis. Protein fractions designated for EM were concentrated using a Pierce Concentrator (100 KDa MWCO, 0.5 ml; Thermo Fisher Scientific) to remove free ubiquitin from the samples.

Ubiquitin ligase assay. Aliquots of DUB-treated or “restored” BRCA1-BARD1 (0.2 mg/ml) were incubated with 10 μM ML364 (USP2 inhibitor) at 37° C. for 1 hour to halt USP2 activity. Samples were then stored overnight at 4° C. The restored BRCA1-BARD1 fractions were added in varying amounts to soluble nuclear extracts prepared from T98G cells. T98G cells naturally produced low levels of BRCA1. Control samples lacked restored BRCA1-BARD1. The reaction mixtures were incubated in a water bath at 37° C. for 1 hour.

Fluorescence microscopy. The production of 8-Oxo-Guanine (8-Oxo-G) DNA lesions was detected by fluorescence imaging as previously described (10, 11). Briefly, HCC1937 cells (about 40,000 cells/chamber) were plated in an eight-chamber slide and incubated at 37° C. and 5% CO₂ overnight. Cells were treated with complete culture media containing 1 mM H₂O₂ for 10-20 minutes to induce oxidative damage in the nucleus. Cells were then wash with 1× PBS and treated with ML364 (7 μM-9 μM) for up to 48 hours at 37° C. and 5% CO₂. Control cells were cultured in media without H₂O₂ and ML364 during the same time period. Cells were washed with standard PBS solution and fixed with 4% paraformaldehyde (Electron Microscopy Sciences) for 15 minutes. The fixed cells were permiabilized by PBS solution containing 0.5% Triton X-100 (Sigma) for 10 minutes. After 1-hour blocking step with PBS supplemented with 10% normal goat serum (Jackson Immuno Research) and 0.2% Triton X-100, cells were incubated with anti-8-OxoG DNA Lesion (483.15; Santa Cruz Biotechnology) at 4° C. overnight. The 8-OxoG DNA lesions were detected with goat anti-mouse IgM-TR (Santa Cruz Biotechnology) and nuclei were stained with Hoechst 33342. An inverted fluorescence microscope (Zeiss Axio Vert.A1; Carl Zeiss Microscopy) was used to image the cells.

Cell viability and proliferation assays. To determine cellular viability, we used the CellTiter 96® AQᵤₑₒᵤₛ One Solution Cell Proliferation Assay (MTS; Promega). HCC1937 were seeded (about 4000 cells per well) in a 96-well plate at 37° C. and 5% CO₂ overnight. Following a 10-minute incubation with culture media containing 1 mM H₂O₂, cells were washed with PBS solution and further treated with 7 μM ML364 for 24 hours at 37° C. with 5% CO₂. Control cells were cultured in media lacking H₂O₂ and/or ML364 during the same time period. Culture media and containing MTS reagent was added to each well. The titer plate was incubated 37° C. and 5% CO₂ for 1-4 hours. Absorbance values were measured at 490 nm using iMark microplate reader (Bio-Rad). MTS was also used to determine cell growth following ML364 treatment. HCC1937 cells were plated (about 1500 cells per well) in a 96-well plate at 37° C. and 5% CO₂ overnight. Cells were treated with DMSO and ML364 at the different doses (2.5 μM-80 μM). After 3 days of incubation, cell viability was measured using the MTS reagent. Cellular survival was calculated by dividing the absorbance of the treated groups by those of the untreated groups (DMSO). Values for the half-maximal inhibitory concentration (IC₅₀) were obtained using Prism software package (GraphPad).

EM specimen preparation and data collection. Aliquots of purified proteins (about 3 μl each of 0.02 mg/ml) were added to the surface of glow-discharged silicon nitride (SiN) microchips or 400-mesh copper EM grids (Ted Pella, Inc.) that were coated with carbon support films. After a 1-minute incubation, the samples were washed with purified water, stained with 0.75% uranyl formate, and allowed to air dry. The EM specimens were viewed using a Spirit BioTwin TEM (Thermo Fisher Scientific) equipped with a LaBe filament and operating at 120 kV under low-dose conditions (<5 electrons/Å₂). Images were recorded using an Eagle 2k HS CCD camera having a pixel size of 30-μm (Thermo Fisher Scientific) at a magnification of ˜68,000× for a sampling of 4.4 Å/pixel.

Image processing routines and difference mapping. A full description of the BRCA1-BARD1 reconstructions was previously reported (10). Briefly, individual particles were selected from EM images and the RELION software package was used to refine and reconstruct density maps using a spherical model. The model aided in allocating alignment parameters to each particle in the first round of refinement. Ten subsequent iterations relied on the experimental data using a regularization parameter of T=4. Standard procedures were used employing a pixel size of 4.4 Å/pixel and a mask of 120 Å. EM maps were examined at similar significance levels and threshold values set in the Chimera program. The FSC-0.5 criteria provided an estimate the resolution of each density map, verified by the RMEASURE program. The structure of the non-ubiquitinated BRCA1_{5382insC}-BARD1 (14.7 Å) contained 4222 particles; ubiquitinated BRCA1_{5382insC}-BARD1 (15.6 Å) contained 4103 particles; restored BRCA1-BARD1 (15.4 Å) contained 4000 particles (10). Difference densities were calculated between the following EM maps: 1) non-ubiquitinated BRCA1_{5382insC}-BARD1 and ubiquitinated BRCA1_{5382insC}-BARD1; 2) ubiquitinated BRCA1_{5382insC}-BARD1 and restored BRCA1-BARD1. The density values for each map were normalized to a common range and differences maps were derived using the DIFFMAP executable, which is publically available. The difference peak in the hotspot region exceeded the 3a-threshold level and was considered significant.

For mutated and wild-type p53 complexes, the same reconstruction and refinement procedures were implemented in the RELION software package, using a resolution-filtered (50 Å) model of the p53 core bound to DNA (pdb code, 2AC0). Class averages and reconstructions for the mutated p53 structure contained 747 particles and the wild-type p53 structure contained 704 particles. C2-symmetry was imposed during refinement bringing the total number of particles to an equivalency of 1494 for the mutated structure and 1408 for the wild-type structure. The maps were masked at about 80 Å in diameter. The Chimera software package was used to visualize the density maps at equivalent contour values. Threshold values are included in each map deposition. We used the 0.5-FSC criterion to determine the resolution of each density map. Particle data was divided into two halves and resolution values for each half converged to a common value. The resolution of the mutated p53 structure was estimated to be 15.5 Å, while the resolution of the wild-type structure was estimated to be 20 Å. These values are in good agreement with independent measurements performed using the RMEASURE program.

Movie production. Supplemental Movies were produced using the Chimera software package. For each movie the EM reconstructions and relevant models were imported and aligned manually within each session. Separate files containing Chimera commands were generated for the reconstructions and models were rotated, rocked, and cross-sectioned. Labeled sites indicate the location of protein or DNA features. For the BRCA1_{5382insC}-BARD1 structures, labels included the ubiquitin site in the hotspot region, along with the RING, and BRCT domains. For the p53_{R306} and p53_{WT} structures, protein domains and DNA are shown in different stages of repair. To produce cross-section views, camera slices were generated using 110 frames and reversed as frame slices were replaced. Each structure was rotated about the x- or y-axis by 1 degree per frame for up to 90 frames. Chimera output for each movie was .mov format.

Results and Discussion.

Movies.

Movie S5. Movie of ubiquitinated BRCA1_{5382insC}-BARD1 isolated from human cancer cells. The 3D reconstruction of the ubiquitinated BRCA1_{5382insC}-BARD1 heterodimer (yellow) is rotated in different orientations to demonstrate the features of the density map. These features include a bulky region in the “hotspot” area that accommodates an ubiquitin model (purple; pdb code, 1UBQ (33)) adjacent to the RING domain. Additional models used to interpret the reconstruction included the BRCA1-BARD1 RING domain (magenta; pdb code, 1JM7(8), and a homology model for the BRCT domain (11) (red). Cross-sections through the reconstruction shows the quality of the model fit in the density.

Movie S6. Movie of restored BRCA1-BARD1 following DUB treatment. The 3D reconstruction of the restored BRCA1-BARD1 heterodimer (green) is shown in a variety of orientations to demonstrate the overall architecture of the EM map. The bulky density in the hotspot region was removed from the deubiquitinated (restored) structure. The absence of the density in this area recapitulates the structural integrity of the unmodified heterodimer (10).

Movie S7. Movie of the mutated p53_{R306} tetramer bound to damaged DNA. The 3D reconstruction (white; about 15.5 Å) of the truncated p53_{R306} tetramer (yellow atomic model; based on pdb code, 2AC0 (21)) is shown in different orientations to demonstrate the protein-DNA engagement. Truncated p53 surrounds a DNA helix (blue) containing a double stranded break (DSB). Cross-sections through the maps and structures demonstrate the quality of the model fit in the EM envelope.

Movie S8. Movie of the wild-type p53 tetramer bound to native DNA during repair. The 3D reconstruction (white; about 20 Å) of the wild-type p53 tetramer (yellow atomic model; based on pdb code, 2AC0 (21)) is shown in different orientations to highlight the continuous density in the DNA (blue) region of the map. This continuity in density can suggest that the wild-type assembly is likely in an active state of repair. Cross-sections through the maps and structures show the quality of the model fit in the EM envelope.

Movie S9. Movie to compare rotational views of the wild-type p53 and mutated p53_{R305} structures. The 3D reconstructions (white) of the wild-type p53 and mutated p53_{R306} assemblies are shown side by side in different rotational views to compare features present in the two structures. Atomic models used to interpret the EM maps include adaptations of the p53 tetramer assembly (based on pdb code, 2AC0 (21)). Each tetramer is bound to a DNA helix that is either damaged (p53_{R306} map) or undergoing a putative repair process (p53_{WT}). Rotational views show a comparable overall fit of the models within each respective density map.

Movie S10. Movie to compare cross-sections through the wild-type p53 and mutated p53_{R306} structures. Similar views of the wild-type p53 and mutated p53_{R306} EM reconstructions (white) shown in a variety of orientations to compare cross-sections through the structures. Atomic models used to interpret the EM maps include adaptations of the p53 tetramer assembly (based on pdb code, 2AC0 (21)). Examining cross-sections through both reconstructions simultaneously highlights the differences in the DNA region, which is continuous in the p53_{WT} structure and fragmented in the mutated p53_{R306} density map.

Structurally correcting mutated BRCA1. To gain insight of how mutations in BRCA1 affect its molecular properties, breast cancer cells that express BRCA1_{5382insC} (HCC1937 line; (17)) were utilized. This mutation truncates the C-terminal domain of BRCA1 by about 10 kDa and it is one of the most common aberrations in hereditary breast cancer worldwide. Cells were cultured under normal conditions at 37° C. with 5% CO₂. Prior to harvesting, cells were treated with 1 mM hydrogen peroxide (H₂O₂) for various time intervals at 37° C. to induce oxidative stress (11, 10) (see Materials and Methods section of this Example). Stressful culture conditions enhanced the formation of ubiquitinated-BRCA1 for downstream analysis.

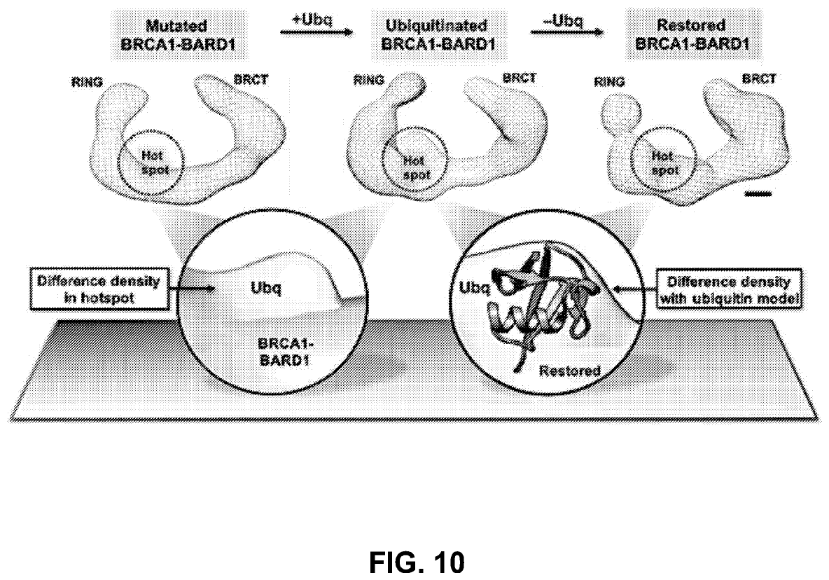

The molecular features of mutated BRCA1-BARD1 purified from breast cancer cells were compared under different treatment conditions. The recently reported electron microscopy (EM) structures (10) were calculated using the RELION software package (18). Under oxidative stress, mutated BRCA1-BARD1 (FIG. 10, gray mesh) gained ubiquitin adducts in a defined hotspot region on the protein. Upon treating ubiquitinated BRCA1 (FIG. 10, yellow mesh; Movie S5) with the deubiquitinating enzyme, Ubiquitin Specific Peptidase 2 (USP2), the bulky density in this hotspot region disappeared. The treated structure is referred to herein as “restored” BRCA1 (FIG. 10, green mesh; Movie S6). The difference density in the hotspot region can accommodate a model for the ubiquitin monomer. The orientation of the model presented here permits it to attach to BRCA1 in a biologically-relevant manner.

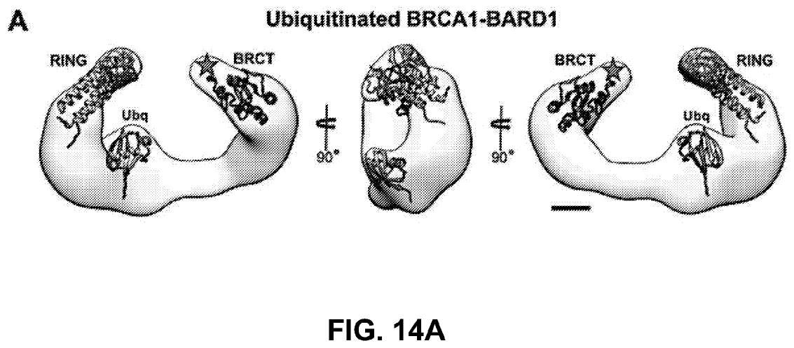

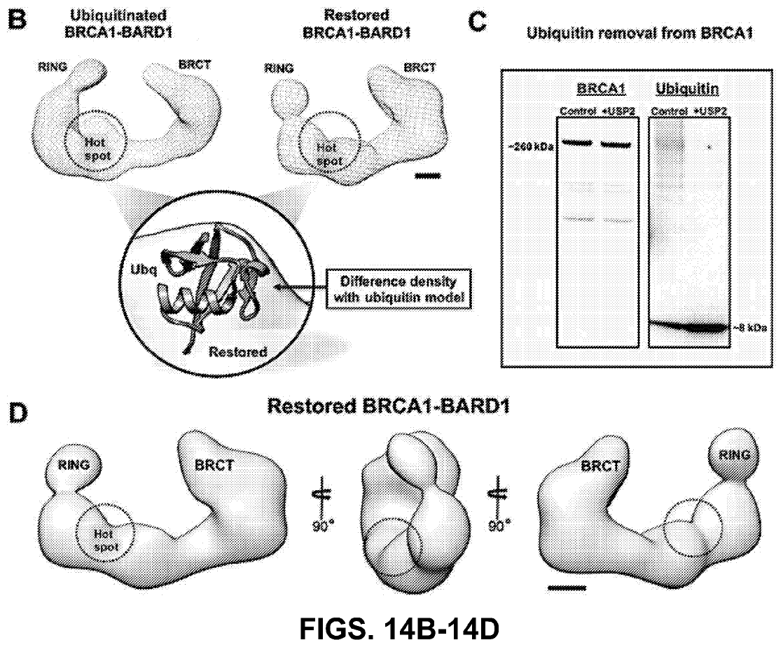

The presence of ubiquitin on mutated BRCA1 was confirmed by SDS-PAGE and Western blot analysis. Ubiquitinated-BRCA1 migrated at about 270 kDa, a rate slower than predicted for the truncated protein (FIGS. 14A-14D). Ubiquitinated samples treated with USP2 showed a shift in the mobility of BRCA1 to about 260 kDa. This change in mobility corresponded with an increase in free ubiquitin at about 8 kDa (FIGS. 14A-14D) as previously noted (10). This rescued form of the protein is referred to herein as “restored BRCA1”. Taken together, these results suggested that we can successfully alter the properties of mutated BRCA1 in vitro. This exciting discovery established the foundation for new functional studies on restored BRCA1.

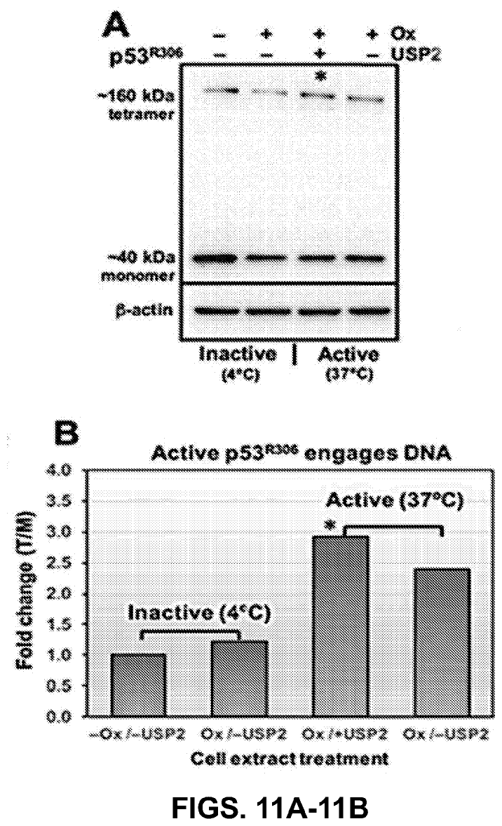

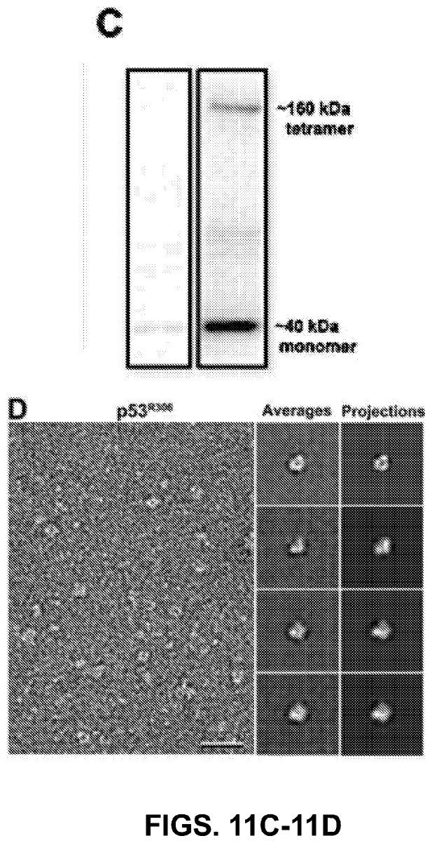

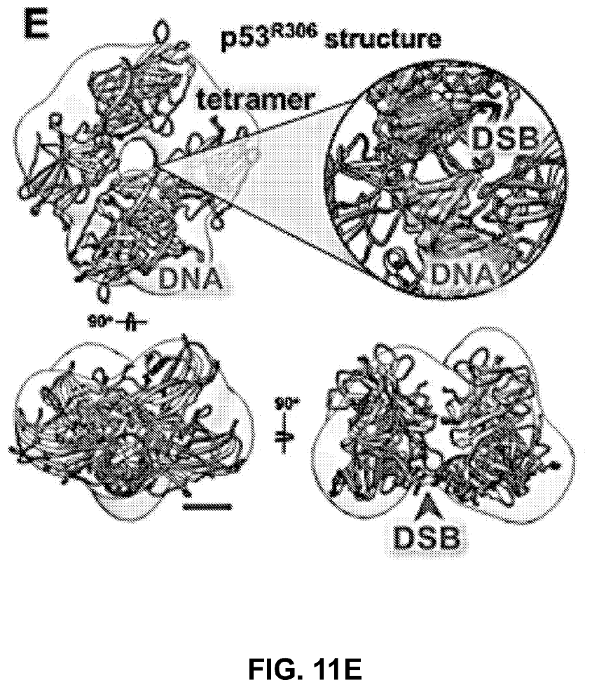

Functionally correcting mutated BRCA1. In the nucleus, p53 is a key participant in DNA damage response—frequently referred to as the “guardian of the genome”. During the life cycle of the protein, modifications to p53 can either stimulate its repair activity or trigger its degradation (19). One of these influential modifications is ubiquitination. As p53 is ubiquitinated by BRCA1 during DNA damage response, it was tested whether restored BRCA1 could function in this capacity. Nuclear extracts were prepared from HCC1937 cells that contained restored BRCA1 and monitored for p53 activation using Western blot detection and quantification. During repair, p53 forms tetramers upon DNA lesions. p53-tetramer formation was looked for in cells receiving oxidative reagents (see e.g. Materials and Methods section of this Example). The HCC1937 cells express a truncated form of p53 (p53_{R306}) with known repair function (20). We compared the ratio ofs:1 p53_{R306} tetramers/monomers (T/M ratio) in nuclear extracts and used this signature to assess DNA engagement (FIGS. 11A and 11B). The T/M ratio increased by about 3-fold or nearly 20% in samples containing restored BRCA1 compared to controls. These results can show p53_{R306} tetramer formation was enhanced in the presence of restored BRCA1.

To better understand the physical nature of the p53_{R306} assemblies, the complexes were purified from H₂O₂-treated HCC1937 cells for additional characterization. Coomassie-stained denaturing gels revealed the purified p53_{R306} monomer (FIG. 11C) and active tetramers were present on Western blots and in EM images (FIGS. 11C and 11D). The tetramer population was concentrated by using Pierce concentrators (PES, 100 kDa MWCO; Thermo Scientific). From the EM images of the tetramers, protein complexes were selected using the RELION software package. An initial model of the p53 tetramer core (pdb code 2AC0; (21)) was used to assist with reconstructing an EM density map that was refined using standard procedures in RELION (see e.g. Materials and Methods section of this Example).

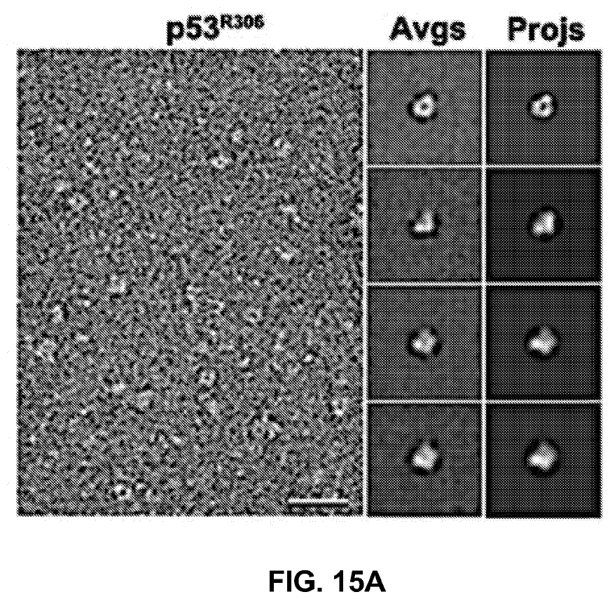

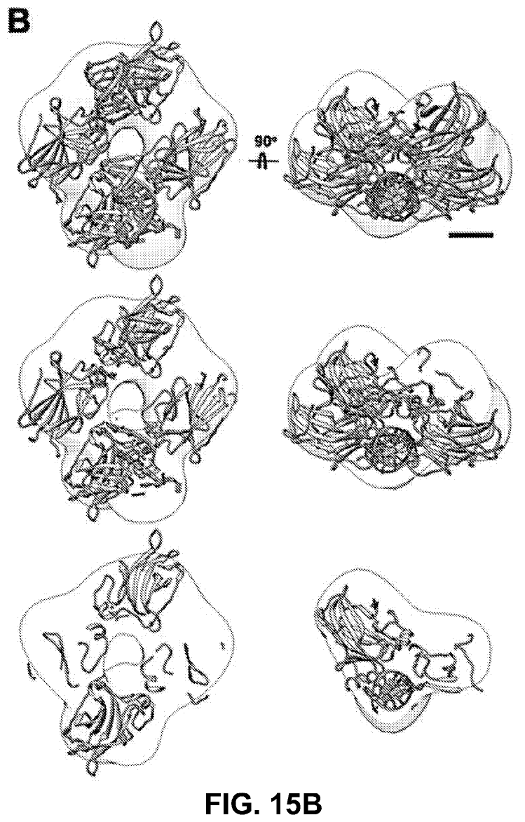

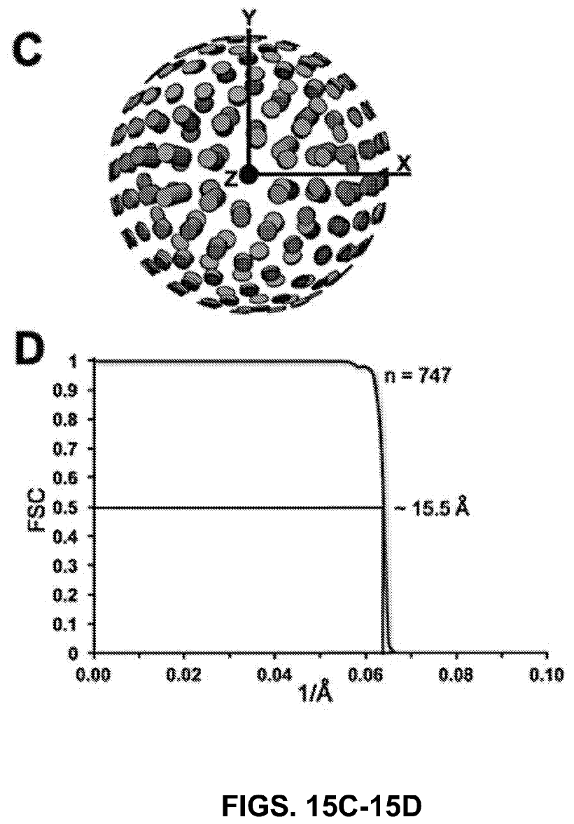

Cross-sections through the 3D reconstruction and model revealed p53_{R306} engaging a native DNA strand having double-stranded breaks (DSB) (FIGS. 11E and 15A-15D; and Movie S7). Projections of the density map were in good agreement with class averages, and the angular orientations of the particle population were not limited in the about 15.5-Å structure (FIGS. 11D and FIGS. 15A-15D). At the current resolution, we cannot distinctly assign ubiquitin density in the p53_{R306} map, although tetramer formation is supported by the biochemical data. The ubiquitin ligase activity of restored BRCA1 on wild-type p53 was evaluated using a different cellular system.

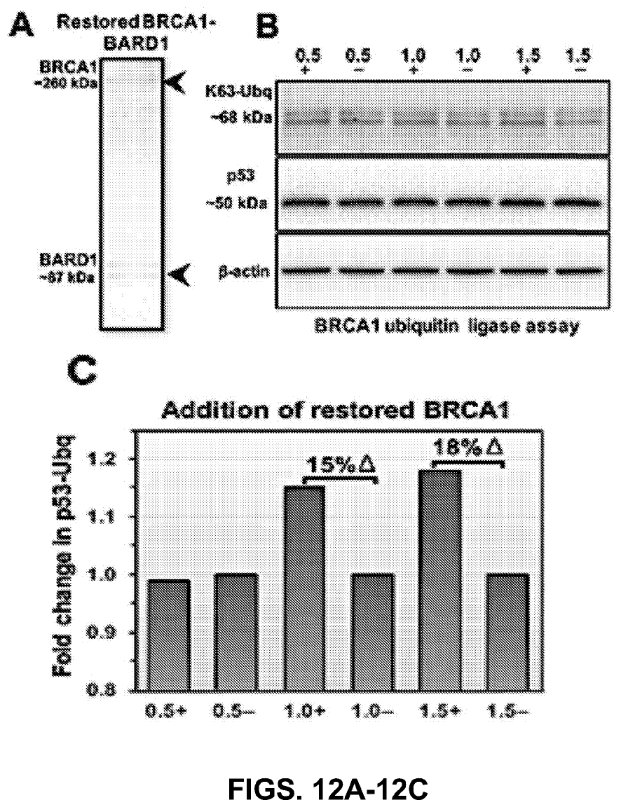

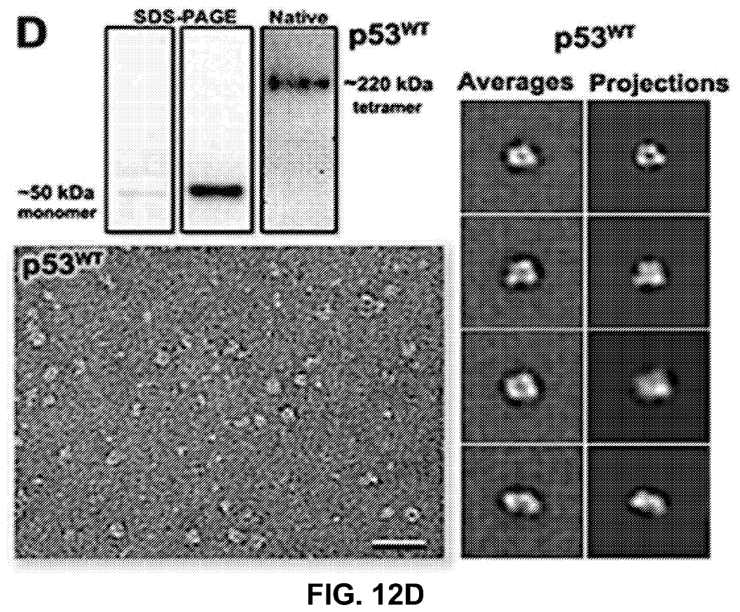

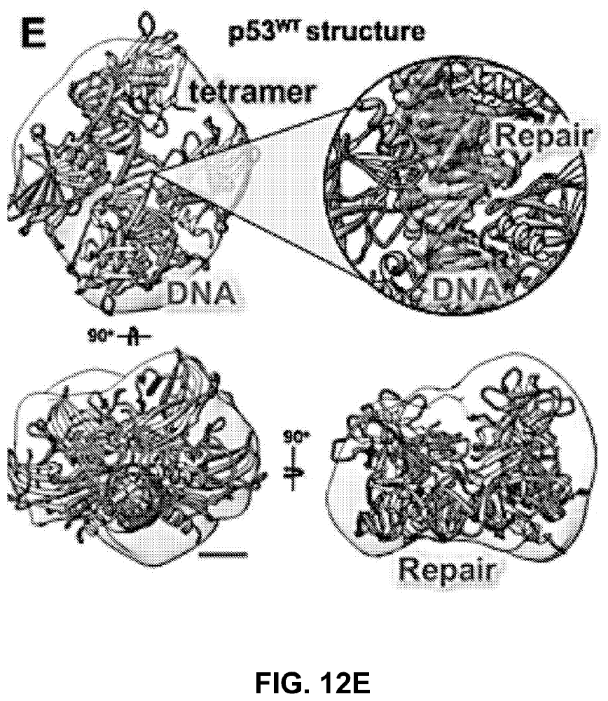

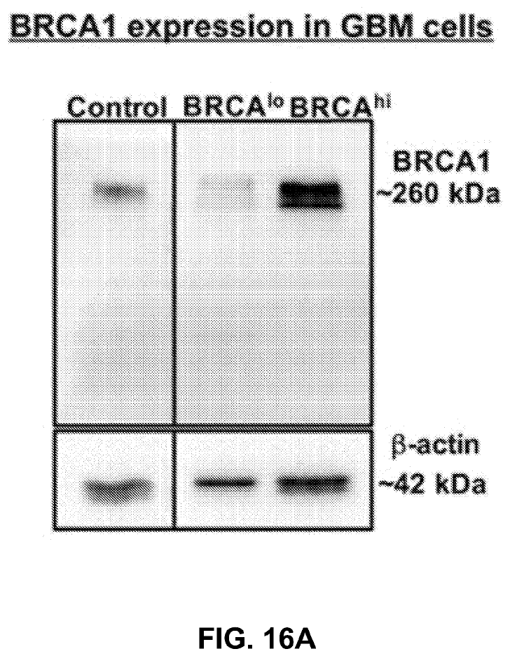

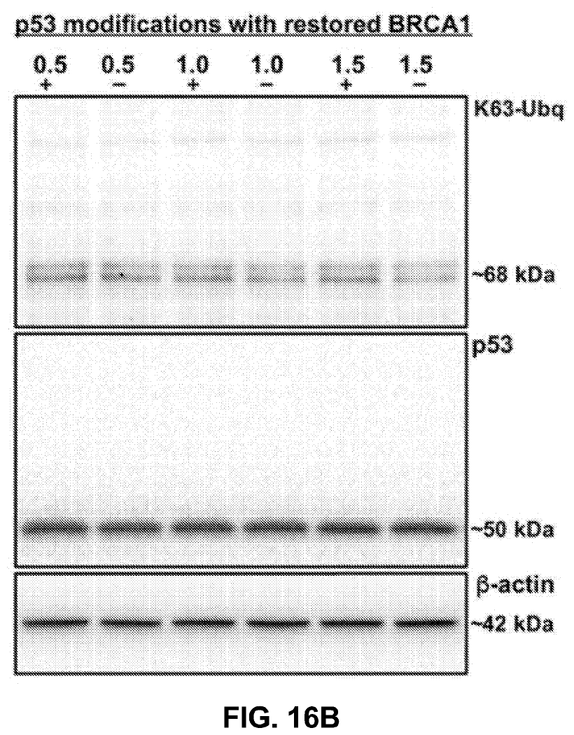

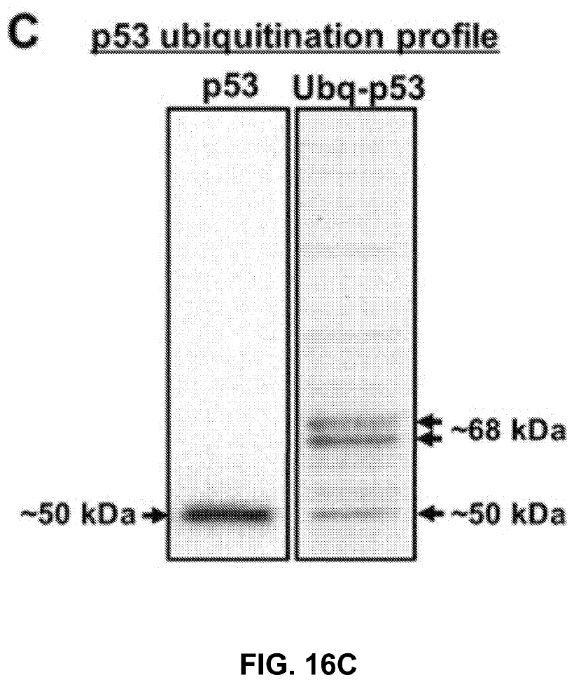

Wild-type p53-caught in the act of repair. The extent to which restored BRCA1 could enhance the ubiquitin ligase activity of cells with inherently low levels of BRCA1 (T98G line) (FIGS. 16A-16C) was evaluated. Quantities of restored BRCA1-BARD1 were added to nuclear reaction mixtures prepared from T98G cells, and changes in ubiquitination were assessed over multiple experiments (FIG. 12A). To simplify the results, modifications to the p53 monomer was first evaluated. According to Western blots, full-length p53 migrated at about 50 kDa while ubiquitnated monomers migrated close to about 68 kDa (FIGS. 12B and 16A-16C). Proteins receiving K63-linked ubiquitin adducts are often involved in autophagy and DNA repair (22), (23), (24). Reaction mixtures receiving restored BRCA1-BARD1 showed increased levels of K63-type ubiquitin adducts on p53 (up to 18%) compared with control samples (FIG. 12C). As the T98G cells naturally produced little BRCA1, we attributed the increase in ubiquitin ligase activity to the restored BRCA1-BARD1 supplement. By comparison, wild-type BRCA1-BARD1 supplementation was observed to increase the ubiquitination of p53 by about 24%. Hence, restored BRCA1 could operate up to 75% of its full capacity.

To visualize differences in the wild-type and mutated p53 structures, wild-type p53 assemblies was biochemically purified from human cancer cells (U87MG line). Monomeric p53 migrated at about 50 kDa on denaturing gels (FIG. 12D). To ensure the presence of p53 tetramers, native gel electrophoresis was performed, which confirmed the tetramers migrated at about 220 kDa. The purified tetramers were concentrated and imaged using EM. Class averages of the wild-type assemblies were larger in diameter (about 80 Å) than their mutated counterparts (about 70 Å), but showed similar overall features. Particles were selected from EM images using the same criteria, model, and reconstruction procedures.

The resulting EM density map (about 20 Å) accommodated the p53 tetramer model with a notable difference in the wild-type structure. The density surrounding the DNA strand was continuous in the wild-type map. (FIG. 12E and 17A-17D; Movie S8). This continuity of density in the wild-type structure can suggest an intact DNA strand, consistent with a state of repair (FIG. 11E; Movies S9 and S10). The same region of density was fragmented in the structure of the mutated complex (FIGS. 11A-11E). Projections of the density map were consistent with experimentally-determined class averages calculated from the overall particle population (FIG. 12D). The reconstructions presented here can demonstrate p53 structures derived from human cancer cells.

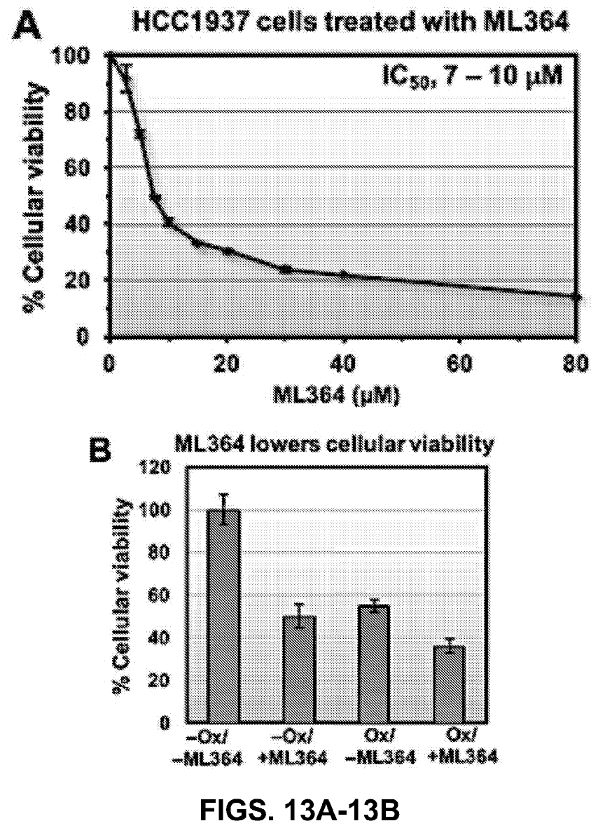

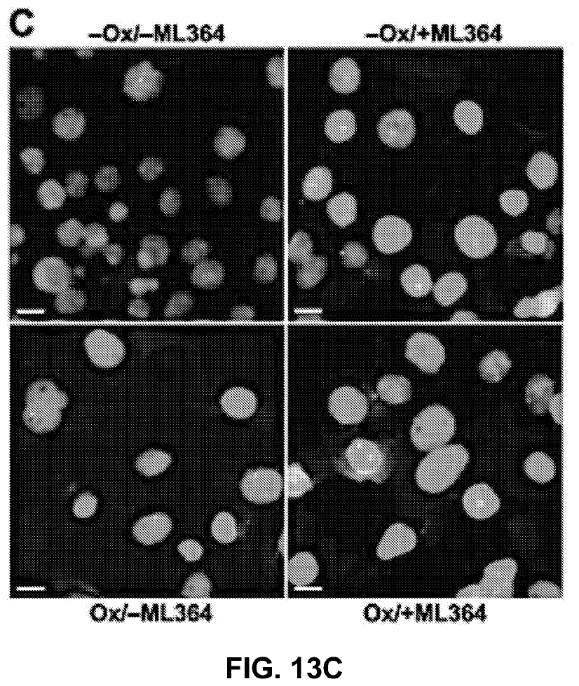

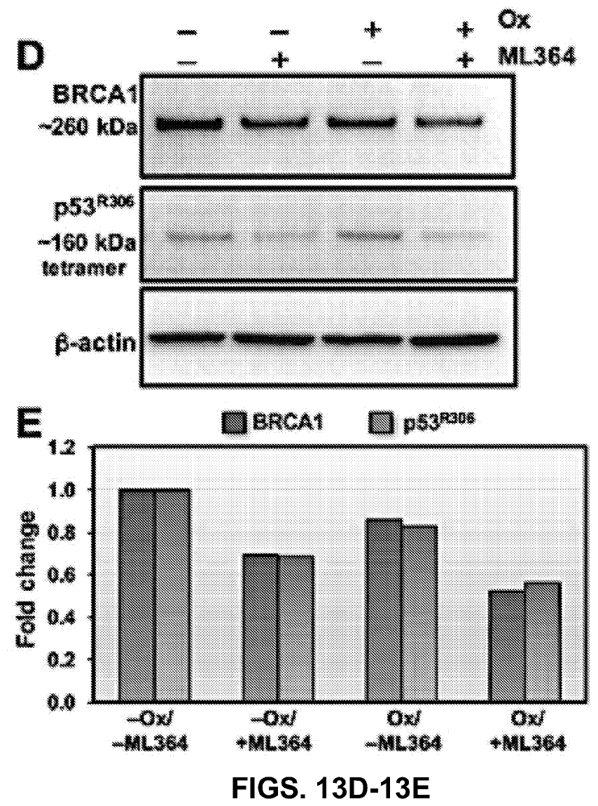

Targeting cancer cells that express mutated BRCA1. As described elsewhere herein, mutated BRCA1 can be fine-tuned in vitro. The application of this aspect of the techology to treat cancer cells was evaluated. Enhancing the activity of tumor suppressors in healthy cells can facilitate their growth and resilience. Conversely, treating cancer cells with drugs that limit DNA repair may lessen their survival. Poly(ADP-Ribose) Polymerase (PARP) inhibitors have been used to treat BRCA1-related cancers (25-28). A limited-repair paradigm approach was developed and used treated breast cancer cells (HCC1937 line) with the DUB inhibitor, ML364 (Axon Ligands™). ML364 is a USP2-specific drug that interferes with cell cycle progression and homologous recombination in colorectal cancer and lymphoma models (29). The half maximal inhibitory concentration (IC₅₀) range for ML364 in HCC1937 cells is about 7-10 μM (FIG. 13A). Cells were treated with 1 mM H₂O₂ for 10 minutes, after which time, the oxidizing agent was removed and cells were then incubated with ML364 in the IC₅₀ range up to 48 hours (FIGS. 18A-18D). Untreated control cells simply received culture media and experiments were performed using four replicates. Oxidative damage has been demonstrated to cause the degradation of BRCA1 (11). Without being bound by theory, the addition of ML364 was believed to enhance these oxidative effects by negatively impacting BRCA1 and p53. The viability of treated cells and untreated cells was quantified using MTS assay measurements. Cells treated with ML364 and H₂O₂ separately showed a decrease in viability by about 50%, while those receiving the combined treatment declined by about 70% (FIG. 13B).

To determine the effect of ML364 on genomic stability, we performed immunofluorescent imaging on HCC1937 breast cancer cells to detect 8-Oxo-Guanine (8-Oxo-G) (FIG. 13C). The nuclear accumulation of 8-Oxo-G in cells indicates deficiencies in DNA damage repair (10, 11). Cells were treated with H₂O₂ to induce oxidative DNA damage. The ML364 drug was then administered to the cells in the IC₅₀ range. The accumulation of 8-Oxo-G was detected using monoclonal antibodies against the substrate and fluorescently-labeled secondary antibodies. Control samples received culture media lacking H₂O₂ or ML364 and showed some inherent DNA damage as anticipated (11). A higher level of 8-Oxo-G accumulation (red punctate) was noted in and around the nucleus (blue fluorescent signal) of cells treated with ML364. Under oxidative stress conditions, there was a minor degree of 8-Oxo-G detected in nuclear region following 10 minutes of treatment. Cells treated with a combination of H₂O₂ and ML364 were observed to have the greatest degree of 8-Oxo-G accumulation and a reduced capacity for DNA damage repair (FIGS. 13C and 18A-18D). To better understand the impact of these treatments on the BRCA1 warning system, we examined changes in repair proteins under each condition.

According to Western blot analysis, BRCA1 and p53 levels decreased by about 30% in ML364-treated cells (FIGS. 13D and 13E). H₂O₂-treated cells lacking ML364 treatment showed a decrease in both proteins by about 15-20%. Cells treated with a combination of H₂O₂ and ML364 showed a remarkable decline (about 50%) in BRCA1 and p53. This trend continued up to 48-hours, although, protein recovery was stronger in H₂O₂-treated cells lacking ML364 (FIGS. 18A-18D). These results suggested that both tumor suppressors were naturally sensitive to the ML364 treatment, particularly under oxidative stress. Our data is consistent with other studies on cancer cells involving the use of DUB inhibitors to promote protein degradation and decrease cellular vitality (29).

As deficiencies in BRCA1 and p53 compromised the cellular warning system, DNA repair response was limited. The build-up of 8-Oxo-G and other toxic species in the nucleus contributed to cellular demise. As cancer cells grow, divide, and produce proteins more rapidly than healthy cells, limiting their repair options poses a powerful therapeutic approach. In this Example, the DUB inhibitors demonstrated anability to kill breast cancer cells heavily reliant upon robust repair activities. These findings also contribute fresh design concepts for small molecules or DUB inhibitors to combat hereditary cancers. Collectively implementing new mechanistic-based strategies introduces promising opportunities to manage disease conditions for individuals with errors in tumour suppressor genes.

Data Availability.

Density maps were refined and reconstructed with RELION software package (https://www2.mrc-Imb.cam.ac.u1c/relion). The Chimera software was used for the visualization and analysis of the density maps (https://www.cgl.ucsf.edu/chimera). The resolution of each density map was verified by RMEASURE (http://grigoriefflab.janelia.org/rmeasure).

Accession Numbers.

EM density maps for wild-type p53 (EMD-8927) and p53_{R306} (EMD-8926) are publically available for download from the EMdatabank (http://www.emdatabank.org/).

EM Databank numbers for the BRCA1 mutant: (EMD-8833), EMD-8834 (wild-type)

### References for Example 2

1. I. Bae et al., (2004). BRCA1 induces antioxidant gene expression and resistance to oxidative stress. Cancer Res., 64, 7893-7909.

2. E. Alli, V. B. Sharma, P. Sunderesakumar, J. M. Ford. (2009). Defective repair of oxidative dna damage in triple-negative breast cancer confers sensitivity to inhibition of poly(ADP-ribose) polymerase. Cancer Res., 69, 3589-3596.

3. D. E. Christensen, P. S. Brzovic, R. E. Klevit. (2007). E2-BRCA1 RING interactions dictate synthesis of mono- or specific polyubiquitin chain linkages. Nat. Struct. Mol. Biol., 14, 941-948.

4. M. D. Stewart et al. (2017). Tuning BRCA1 and BARD1 activity to investigate RING ubiquitin ligase mechanisms. Protein Sci., 26, 475-483.

5. L. S. Friedman et al. (1994). Confirmation of BRCA1 by analysis of germline mutations linked to breast and ovarian cancer in ten families. Nat. Genet., 8, 399-404.

6. M. C. King, J. H. Marks, J. B. Mandell. (2003). Breast and ovarian cancer risks due to inherited mutations in BRCA1 and BRCA2. Science, 302, 643-646.

7. A. di Masi, F. Gullotta, V. Cappadonna, L. Leboffe, P. Ascenzi. (2011). Cancer Predisposing Mutations in BRCT Domains. lubmb Life, 63, 503-512.

8. P. S. Brzovic, J. E. Meza, M. C. King, R. E. Klevit. (2001). BRCA1 RING domain cancer-predisposing mutations. Structural consequences and effects on protein-protein interactions. J. Biol. Chem., 276, 41399-41406.

9. S. Sankaran, L. M. Starita, A. M. Simons, J. D. Parvin. (2006). Identification of domains of BRCA1 critical for the ubiquitin-dependent inhibition of centrosome function. Cancer Res., 66, 4100-4107.

10. Y. Liang et al. (2017). Structural analysis of BRCA1 reveals modification hotspot. Science Adv., 3, e1701386.

11. B. L. Gilmore et al. (2017). Molecular Analysis of BRCA1 in Human Breast Cancer Cells Under Oxidative Stress. Sci. Rep., 7, 43435.

12. H. Zhang et al. (1998). BRCA1 physically associates with p53 and stimulates its transcriptional activity. Oncogene, 16, 1713-1721.

13. Buck, M. (2008). A novel domain of BRCA1 interacts with p53 in breast cancer cells. Cancer Lett., 268, 137-145.

14. D. Malkin et al. (1990). Germ line p53 mutations in a familial syndrome of breast cancer, sarcomas, and other neoplasms. Science, 250, 1233-1238.

15. N. Rivlin, R. Brosh, M. Oren, V. Rotter. (2011). Mutations in the p53 Tumor Suppressor Gene: Important Milestones at the Various Steps of Tumorigenesis. Genes Cancer, 2, 466-474.

16. P. A. Muller, K. H. Vousden. (2013). p53 mutations in cancer. Nat. Cell. Biol., 15, 2-8.

17. G. E. Tomlinson et al. (1998). Characterization of a breast cancer cell line derived from a germ-line BRCA1 mutation carrier. Cancer Res., 58, 3237-3242.

18. S. H. Scheres. (2012). RELION: implementation of a Bayesian approach to cryo-EM structure determination. J. Struct. Biol., 180, 519-530.

19. E. Powell, D. Piwnica-Worms, H. Piwnica-Worms. (2014). Contribution of p53 to metastasis. Cancer Discov., 4, 405-414.

20. R. L. Siegel, K. D. Miller, A. Jemal, Cancer statistics, 2016. (2016). CA Cancer J. Clin., 66, 7-30.

21. M. Kitayner et al. (2006). Structural basis of DNA recognition by p53 tetramers. Mol. Cell, 22, 741-753.

22. J. M. Tan et al. (2008). Lysine 63-linked ubiquitination promotes the formation and autophagic clearance of protein inclusions associated with neurodegenerative diseases.

Hum. Mol. Genet., 17, 431-439.

23. J. A. Nathan, H. T. Kim, L. Ting, S. P. Gygi, A. L. Goldberg. (2013). Why do cellular proteins linked to K63-polyubiquitin chains not associate with proteasomes? Embo J., 32, 552-565.

24. L. van Cuijk, W. Vermeulen, J. A. Marteijn. (2014). Ubiquitin at work: the ubiquitous regulation of the damage recognition step of NER. Exp. Cell Res., 329, 101-109.

25. H. Farmer et al. (2005). Targeting the DNA repair defect in BRCA mutant cells as a therapeutic strategy. Nature, 434, 917-921.

26. A. N. Tutt et al. (2005). Exploiting the DNA repair defect in BRCA mutant cells in the design of new therapeutic strategies for cancer. Cold Spring Harb. Symp. Quant. Biol., 70, 139-148.

27. P. C. Fong et al. (2009). Inhibition of poly(ADP-ribose) polymerase in tumors from BRCA mutation carriers. N. Engl. J. Med., 361, 123-134.

28. J. J. J. Geenen, S. C. Linn, J. H. Beijnen, J. H. M. Schellens. (2018). PARP Inhibitors in the Treatment of Triple-Negative Breast Cancer. Clin. Pharmacokinet, 57, 427-437.

29. M. I. Davis et al. (2016). Small Molecule Inhibition of the Ubiquitin-specific Protease USP2 Accelerates cyclin D1 Degradation and Leads to Cell Cycle Arrest in Colorectal Cancer and Mantle Cell Lymphoma Models. J. Biol. Chem., 291, 24628-24640.

30. T. A. Yap, S. K. Sandhu, C. P. Carden, J. S. de Bono. (2011). Poly(ADP-ribose) polymerase (PARP) inhibitors: Exploiting a synthetic lethal strategy in the clinic. CA Cancer J. Clin., 61, 31-49.

31. M. J. O'Connor. (2015). Targeting the DNA Damage Response in Cancer. Mol. Cell, 60, 547-560.

32. E. Alli, J. M. Ford. (2012). Breast cancers with compromised DNA repair exhibit selective sensitivity to elesclomol. DNA Repair (Amst), 11, 522-524.

33. S. Vijay-Kumar, C. E. Bugg, W. J. Cook. (1987). Structure of ubiquitin refined at 1.8 A resolution. J. Mol. Biol., 194, 531-544.

34. E. F. Pettersen et al. (2004). USCF Chimera—a visualization system for exploratory research and analysis. J. Comput. Chem., 25, 1605-1612.

35. D. Sousa, N. Grigorieff. (2007). Ab initio resolution measurement for single particle structures. J. Struct. Biol., 157, 201-210.

36. J. Frank, (2006). Three-Dimensional electron microscopy of macromolecular assemblies. Oxford Univ Press, Inc. New York.

We claim:

1. A method of treating a cancer or symptom thereof in a subject in need thereof, the method comprising:
administering an amount of a BRCA1 modulating compound or pharmaceutical formulation thereof to the subject in need thereof.

2. The method of claim 1, wherein the BRCA1 modulating compound is a deubiquitinase.

3. The method of claim 2, wherein the deubiquitinase is selected from the group consisting of: USP2, USP1, USP3, USP4, USP5, USP6, USP7, USP8, USP9X, USP9Y, USP10, USP11, USP12, USP13, USP14, USP15, USP16, USP17, USP17L2, USP17L3, USP17L4, USP17L5, USP17L7, USP17L8, USP18, USP19, USP20, USP21, USP22, USP23, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, USP46, OTUB1, OTUB2, ATXN3, ATXN3L, BAP1, UCHL1, UCHHL3, UCHL5, and any combination thereof.

5. The method of claim 1, wherein the BRCA1 modulating compound is a deubiquitinase inhibitor.

6. The method of claim 5, wherein the deubiquitinase inhibitor is selected from the group consisting of: ML364, P022077, P5091, Cpd 14, P22077, HBX 41,108, HBX-19,818, HBX-28,258, HBX 90,397, Ethyloxyimino-9H-indeno [1,2-b] pyrazine-2,3-dicarbonitrile, IU1, Isatin O-acyl oxime deriatives, LDN91946, LS1, NSC112200, NSC267309, PR-619, 15-Deoxy-α_{12,14} prostaglandin J2, b-AP15, RA-9, F6, G5, WP1130, Eeyarestatin-1, Curcumin, AC17, Gambogic acid, LDN-57444, GW7647, pimozide, 12Δ-PGJ2, AM146, RA-14, betulinic acid and any combination thereof.

7. The method of any one of claims 5-6, wherein the method further comprises administering a compound to the subject that increases the oxidative stress of a cell or a population thereof in the subject.

8. The method of claim 7, wherein the compound that increases the oxidative stress of a cell or a population thereof is hydrogen peroxide.

9. The method of any one of claims 1-8, wherein the cancer is a breast cancer, ovarian cancer, pancreatic cancer, a brain cancer, or a combination thereof.

10. The method of any one of claims 1-9, wherein the cancer is a cancer that has or is at least in part caused by a mutated BRCA1.

11. The method of claim 10, wherein the cancer is a cancer that has or is at least in part caused by a BRCA1_{5382insC} mutation.

12. The method of any one of claims 1-11, wherein the amount of BRCA1 modulating compound or formulation thereof ranges from about 0.1 μg/kg to about 1000 mg/kg.

13. Use of a BRCA1 modulating compound for the treatment of a cancer or a symptom thereof.

14. Use of a BRCA1 modulating compound in the manufacture of a medicament for treatment of cancer or a symptom thereof.

15. A pharmaceutical formulation comprising:
an effective amount of a BRCA1 modulating compound; and
a pharmaceutically acceptable carrier.

16. The pharmaceutical formulation of claim 15, wherein the BRCA1 modulating compound is a deubiquitinase.

17. The pharmaceutical formulation of claim 16, wherein the deubiquitinase is selected from the group consisting of: USP2, USP1, USP3, USP4, USP5, USP6, USP7, USP8, USP9X, USP9Y, USP10, USP11, USP12, USP13, USP14, USP15, USP16, USP17, USP17L2, USP17L3, USP17L4, USP17L5, USP17L7, USP17L8, USP18, USP19, USP20, USP21, USP22, USP23, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, USP46, OTUB1, OTUB2, ATXN3, ATXN3L, BAP1, UCHL1, UCHHL3, UCHL5, and any combination thereof.

18. The pharmaceutical formulation of claim 15, wherein the BRCA1 modulating compound is a deubiquitinase inhibitor.

19. The pharmaceutical formulation of claim 18, wherein the deubiquitinase inhibitor is selected from the group consisting of: ML364, P022077, P5091, Cpd 14, P22077, HBX 41,108, HBX-19,818, HBX-28,258, HBX 90,397, Ethyloxyimino-9H-indeno [1,2-b] pyrazine-2,3-dicarbonitrile, IU1, Isatin O-acyl oxime deriatives, LDN91946, LS1, NSC112200, NSC267309, PR-619, 15-Deoxy-α_{12,14} prostaglandin J2, b-AP15, RA-9, F6, G5, WP1130, Eeyarestatin-1, Curcumin, AC17, Gambogic acid, LDN-57444, GW7647, pimozide, 12Δ-PGJ2, AM 146, RA-14, betulinic acid and any combination thereof.

20. The pharmaceutical formulation of any one of claims 18-19, wherein the pharmaceutical formulation further comprises an amount of a compound capable of increasing oxidative stress in a population of cells in the subject.
